SEQUENCE LISTING (17 sequences)

SEQ ID NO: 1 - protein, 5 aa, Artificial Sequence
  Synthetic Construct
DYGMV

SEQ ID NO: 2 - protein, 17 aa, Artificial Sequence
  Synthetic Construct
FISSGSSTVYYADTMKG

SEQ ID NO: 3 - protein, 10 aa, Artificial Sequence
  Synthetic Construct
RNYDDWYFDV

SEQ ID NO: 4 - protein, 10 aa, Artificial Sequence
  Synthetic Construct
SASSSVTYMY

SEQ ID NO: 5 - protein, 7 aa, Artificial Sequence
  Synthetic Construct
RTSDLAS

SEQ ID NO: 6 - protein, 9 aa, Artificial Sequence
  Synthetic Construct
QHYHSYPLT

SEQ ID NO: 7 - protein, 119 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSS

SEQ ID NO: 8 - protein, 106 aa, Artificial Sequence
  Synthetic Construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIK

SEQ ID NO: 9 - protein, 448 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 213 aa, Artificial Sequence
  Synthetic Construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 445 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 13 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 14 - protein, 233 aa, Homo sapiens
MLNLLLLALPVLASRAYAAPAPGQALQRVGIVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQVATAPHTFPAPS

SEQ ID NO: 15 - protein, 245 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 16 - protein, 256 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

SEQ ID NO: 17 - protein, 256 aa, Homo sapiens
IVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

(19) United States
(12) Patent Application Publication
(10) Pub. No.: US 2022/0348687 A1
(43) Pub. Date: Nov. 3, 2022

(54) DOSING FOR ANTI-TRYPTASE ANTIBODIES

(71) Applicant: Genentech, Inc., South San Francisco, CA (US)

(72) Inventors: Joseph Haw-Ling LIN, San Mateo, CA (US); Ryan Patrick OWEN, Millbrae, CA (US); Sharon Marie RYMUT, San Francisco, CA (US); Siddharth SUKUMARAN, Foster City, CA (US)

(21) Appl. No.: 17/761,727

(22) PCT Filed: Sep. 18, 2020
(86) PCT No.: PCT/US2020/051416

§ 371 Date: Mar. 18, 2022

Related U.S. Application Data
(60) Provisional application No. 62/903,409, filed on Sep. 20, 2019.

(51) Int. Cl. C07K 16/40; A61P 11/06

(52) U.S. Cl. CPC C07K 16/40; A61P 11/06; A61K 2039/505

(57) ABSTRACT

The present invention features, inter alia, methods of treating a patient having asthma by administering an anti-tryptase antibody (e.g., anti-tryptase beta antibody) to the patient, anti-tryptase antibodies (e.g., anti-tryptase beta antibodies) for use in treating asthma, and uses of anti-tryptase antibodies (e.g., anti-tryptase beta antibodies), e.g., in the manufacture of medicaments for treating asthma.

### SEQUENCE LISTING

The instant application contains a Sequence Listing which has been submitted electronically in ASCII format and is hereby incorporated by reference in its entirety. Said ASCII copy, created on Aug. 14, 2020, is named 50474-204WO2_Sequence_Listing_08.14.20_ST25 and is 26,856 bytes in size.

### FIELD OF THE INVENTION

The present disclosure relates to methods of treating asthma and related compositions and uses.

### BACKGROUND

Asthma is a chronic inflammatory disease of the airways with an increasing worldwide incidence. Approximately 250,000 people die prematurely each year as a result of asthma. The pathophysiology of the disease is characterized by variable airflow obstruction, airway inflammation, mucus hypersecretion, and subepithelial fibrosis. Clinically, patients may present with cough, wheezing, and shortness of breath. Substantial evidence indicates that asthma is not a uniform condition, and there is considerable heterogeneity in clinical characteristics, severity of disease, and underlying biology. The best characterized subtypes consist of those patients in whom the disease is driven by IgE and cytokines expressed by Type 2 T-helper cells and Type 2 innate-lymphoid cells, namely interleukin (IL)-4, IL-5, and IL-13; allergic disease and peripheral eosinophilia are common features.

Despite the development of effective controller therapies for asthma, including inhaled corticosteroids, long-acting beta agonists, and other controller medications, a substantial proportion of patients continue to have uncontrolled symptoms, airflow obstruction, and exacerbations. Improved therapies for asthma are still being sought.

### SUMMARY OF THE INVENTION

The present invention features, inter alia, methods of treating a patient having asthma (e.g., moderate asthma (e.g., moderate asthma that remains uncontrolled despite standard-of-care therapy), severe asthma (e.g., severe asthma that remains uncontrolled despite standard-of-care therapy), allergic asthma, or atopic asthma (e.g., mild atopic asthma)), anti-tryptase antibodies (e.g., anti-tryptase beta antibodies) for use in treating asthma, uses of anti-tryptase antibodies (e.g., anti-tryptase beta antibodies), e.g., in the manufacture of medicaments for treating asthma, as well as related kits and articles of manufacture.

In one aspect, the disclosure features an method of treating a patient having asthma, the method comprising administering to a patient having asthma an anti-tryptase beta antibody in a dosing regimen comprising a dosing cycle, wherein the dosing cycle comprises a first dose (C1D1) of the anti-tryptase beta antibody selected from 300 mg intravenously (IV), 450 mg IV, 750 mg SC, 900 mg IV, 1350 mg IV, 1800 mg IV, or 3600 mg IV, wherein the anti-tryptase beta antibody comprises the following six complementarity determining regions (CDRs): (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the disclosure features an anti-tryptase beta antibody for use in treating a patient having asthma, wherein the anti-tryptase beta antibody is for administration to a patient having asthma in a dosing regimen comprising a dosing cycle, wherein the dosing cycle comprises a first dose (C1D1) of the anti-tryptase beta antibody selected from 300 mg IV, 450 mg IV, 750 mg SC, 900 mg IV, 1350 mg IV, 1800 mg IV, or 3600 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the disclosure features the use of an anti-tryptase beta antibody in the manufacture of a medicament for treating a patient having asthma, wherein the medicament is for administration to a patient having asthma in a dosing regimen comprising a dosing cycle, wherein the dosing cycle comprises a first dose (C1D1) of the anti-tryptase beta antibody selected from 300 mg IV, 450 mg IV, 750 mg SC, 900 mg IV, 1350 mg IV, 1800 mg IV, or 3600 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the disclosure features a method of treating a patient having asthma, the method comprising administering to a patient having asthma an anti-tryptase beta antibody in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the disclosure features an anti-tryptase beta antibody for use in treating a patient having asthma, wherein the anti-tryptase beta antibody is for administration to a patient having asthma in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In some aspects, the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

In some aspects, the VH domain comprises the amino acid sequence of SEQ ID NO: 7.

In some aspects, the VL domain comprises the amino acid sequence of SEQ ID NO: 8.

In some aspects, the VH domain comprises the amino acid sequence of SEQ ID NO: 7 and the VL domain comprises the amino acid sequence of SEQ ID NO: 8.

In some aspects, the antibody comprises (a) a heavy chain comprising the amino acid sequence of SEQ ID NO: 9 and (b) a light chain comprising the amino acid sequence of SEQ ID NO: 10.

In some aspects, the antibody comprises (a) a heavy chain comprising the amino acid sequence of SEQ ID NO: 11 and (b) a light chain comprising the amino acid sequence of SEQ ID NO: 10.

In some aspects, the anti-tryptase antibody is MTPS9579A.

In some aspects, the C1D1 is 300 mg IV.

In some aspects, the C1D1 is 450 mg IV.

In some aspects, the C1D1 is 750 mg SC.

In some aspects, SC administration is performed using a pump.

In some aspects, the pump is a patch pump.

In some aspects, the C1D1 is 900 mg IV.

In some aspects, the C1D1 is 1350 mg IV.

In some aspects, the C1D1 is 1800 mg IV.

In some aspects, the C1D1 is 3600 mg IV.

In some aspects, the dosing cycle further comprises a second dose (C1D2) and a third dose (C1D3) of the anti-tryptase beta antibody, wherein the C1D2 and the C1D3 are each equal to the C1D1.

In some aspects, the doses of the dosing cycle are administered to the subject every four weeks (q4w).

In some aspects, the dosing cycle has a length of about 57 days.

In some aspects, the C1D1 is administered on Day 1 of the dosing cycle, the C1D2 is administered on Day 29 of the dosing cycle, and the C1D3 is administered on Day 57 of the dosing cycle.

In some aspects, the dosing regimen consists of one dosing cycle.

In some aspects, the asthma is severe asthma, allergic asthma, or atopic asthma.

In some aspects, the severe asthma is uncontrolled despite standard-of-care therapy.

In some aspects, the asthma is moderate to severe asthma.

In some aspects, the patient is receiving daily inhaled corticosteroid therapy and at least one of the following controller medications: a long-acting β-agonist (LABA), a leukotriene modulator, a long-acting muscarinic antagonist (LAMA), or a long-acting theophylline preparation.

In some aspects, the leukotriene modulator is a leukotriene modifier (LTM) or leukotriene receptor antagonist (LTRA).

In another aspect, the disclosure features a kit comprising an anti-tryptase beta antibody and instructions to administer the anti-tryptase beta antibody to a patient having asthma in accordance with any one of the methods described herein.

### BRIEF DESCRIPTION OF THE DRAWINGS

FIG. 1 is a schematic diagram of the study design of the GA40396 Phase I clinical study. ^{a}Sentinal dosing was used in all single ascending dose (SAD) cohorts. ^{b}See Example 1 for description of Optional SAD or multiple ascending dose (MAD) cohorts. PK, pharmacokinetic; PD, pharmacodynamic, ADA, anti-drug antibody. FIG. 2 is a graph showing mean (±standard deviation (SD)) serum MTPS9579A concentration-time profiles in healthy subjects after subcutaneous (SC) administration of 30, 100, or 300 mg MTPS9579A, or intravenous (IV) administration of 300, 900, 1800, or 3600 mg MTPS9579A on Day 1 in the SAD portion of Study GA40396. LLOQ, lower limit of quantification.

FIG. 3 is a graph showing mean (±SD) serum MTPS9579A concentration-time profiles in healthy subjects after SC administration of 150, 300, or 750 mg MTPS9579A or IV administration of 1350 or 3600 mg MTPS9579A on Days 1, 29, and 57 in the MAD portion of Study GA40396 (N=8; all dose cohorts). Q4W, every 4 weeks.

FIG. 4 is a series of graphs showing the nasal active tryptase concentration-time profile in each healthy subject after SC administration of 30, 100, or 300 mg MTPS9579A, or IV administration of 300, 900, 1800, or 3600 mg MTPS9579A, or placebo on Day 1 in the SAD portion of Study GA40396.

FIG. 5 is a series of graphs showing the nasal active tryptase concentration-time profile in each healthy subject after SC administration of 150, 300, or 750 mg MTPS9579A, or IV administration of 1350, or 3600 mg MTPS9579A, or placebo Q4W in the MAD portion of Study GA40396.

FIG. 6 is a series of graphs showing the nasal total tryptase concentration-time profile in each healthy subject after SC administration of 30, 100, or 300 mg MTPS9579A, or IV administration of 300, 900, 1800, or 3600 mg MTPS9579A, or placebo on Day 1 in the SAD portion of Study GA40396.

FIG. 7 is a series of graphs showing the nasal total tryptase concentration-time profile in each healthy subject after SC administration of 150, 300, or 750 mg MTPS9579A, or IV administration of 1350, or 3600 mg MTPS9579A, or placebo Q4W in the MAD portion of Study GA40396. FIG. 8 is a series of graphs showing the serum total tryptase concentration-time profile in each healthy subject after SC administration of 30, 100, or 300 mg MTPS9579A, or IV administration of 300, 900, 1800, or 3600 mg MTPS9579A, or placebo on Day 1 in the SAD portion of Study GA40396.

FIG. 9 is a series of graphs showing the serum total tryptase concentration-time profile in each healthy subject after SC administration of 150, 300, or 750 mg MTPS9579A, or IV administration of 1350, or 3600 mg MTPS9579A, or placebo Q4W in the MAD portion of Study GA40396.

FIGS. 10A-10D are a series of graphs showing mean (±SD) serum MTPS9579A concentration over time (log scale) for SC cohorts in the SAD portion of Study GA40396 (FIG. 10A), for IV cohorts in the SAD portion of Study GA40396 (FIG. 10B), for SC cohorts in the MAD portion of Study GA40396 (FIG. 10C), and for IV cohorts in the MAD portion of Study GA40396 (FIG. 10D). FIG. 11 is a schematic diagram of the study design of the GA41003 Phase Ic clinical study. ICS, inhaled corticosteroids; R, randomization. ^{a}The timing of bronchoscopy 2 may be modified after review of preliminary data.

FIG. 12 is a schematic diagram of the study design of the GB41149 Phase Ila clinical study. EOS, end of study; EOT, end of treatment; F/U, safety follow-up; PBO, placebo. ^{a}Screening period is 12-28 days.

### DETAILED DESCRIPTION OF ASPECTS OF THE INVENTION

### I. Introduction

The present invention provides therapeutic methods and compositions for asthma (e.g., moderate asthma (e.g., moderate asthma that remains uncontrolled despite standard-of-care therapy), severe asthma (e.g., severe asthma that remains uncontrolled despite standard-of-care therapy), allergic asthma, or atopic asthma (e.g., mild atopic asthma)). The present invention is based, at least in part, on the discovery that anti-tryptase antibodies, including MTPS9579A, can have unexpectedly low maximum serum concentration (Cₘₐₓ) and short mean half-life values upon administration to a human, and further, that anti-tryptase antibodies, including MTPS9579A, can be safely administered to humans in dosing regimens that involve administration of relatively high antibody doses. Moreover, as demonstrated herein, the dosing regimens disclosed herein inhibit active tryptase, e.g., in the upper airway of humans. It is expected that the dosing regimens disclosed herein will be effective in treating asthma.

### II. Definitions

The term “about,” as used herein, refers to the usual error range for the respective value readily known to the skilled person in this technical field. Reference to “about” a value or parameter herein includes (and describes) aspects that are directed to that value or parameter per se.

As used herein, “tryptase” refers to any native tryptase from any vertebrate source, including mammals such as primates (e.g., humans) and rodents (e.g., mice and rats), unless otherwise indicated. Tryptase is also known in the art as mast cell tryptase, mast cell protease II, skin tryptase, lung tryptase, pituitary tryptase, mast cell neutral proteinase, and mast cell serine proteinase II. The term “tryptase” encompasses tryptase alpha (encoded in humans by TPSAB1), tryptase beta (encoded in humans by TPSAB1 and TPSB2; see below), tryptase delta (encoded in humans by TPSD1), tryptase gamma (encoded in humans by TPSG1), and tryptase epsilon (encoded in humans by PRSS22). Tryptase alpha (α), beta (β), and gamma (γ) proteins are soluble, whereas tryptase epsilon (ϵ) proteins are membrane anchored. Tryptase beta and gamma are active serine proteases, although they have different specificities. Tryptase alpha and delta (δ) proteins are largely inactive proteases as they have residues in critical position that differ from typical active serine proteases. An exemplary tryptase alpha full-length protein sequence can be found under NCBI Gen Bank Accession No. ACZ98910.1. Exemplary tryptase gamma full-length protein sequences can be found under Uniprot Accession No. Q9NRR2 or GenBank Accession Nos. Q9NRR2.3, AAF03695.1, NP_036599.3 or AAF76457.1. Exemplary tryptase delta full-length protein sequences can be found under Uniprot Accession No. Q9BZJ3 or GenBank Accession No. NP_036349.1. Several tryptase genes are clustered on human chromosome 16p13.3. The term encompasses “full-length,” unprocessed tryptase as well as any form of tryptase that results from processing in the cell. Tryptase beta is the main tryptase expressed in mast cells, while tryptase alpha is the main tryptase expressed in basophils. Tryptase alpha and tryptase beta typically include a leader sequence of approximately 30 amino acids and a catalytic sequence of approximately 245 amino acids (see, e.g., Schwartz, Immunol. Allergy Clin. N. Am. 26:451-463, 2006).

As used herein, “tryptase beta” refers to any native tryptase beta from any vertebrate source, including mammals such as primates (e.g., humans) and rodents (e.g., mice and rats), unless otherwise indicated. Tryptase beta is a serine protease that is a major constituent of mast cell secretory granules. As used herein, the term encompasses tryptase beta 1 (encoded by the TPSAB1 gene, which also encodes tryptase alpha 1), tryptase beta 2 (encoded by the TPSB2 gene), and tryptase beta 3 (also encoded by the TPSB2 gene). An exemplary human tryptase beta 1 sequence is shown in SEQ ID NO: 12 (see also GenBank Accession No. NP_003285.2). An exemplary human tryptase beta 2 sequence is shown in SEQ ID NO: 13 (see also GenBank Accession No. AAD13876.1). An exemplary human tryptase beta 3 sequence is shown in SEQ ID NO: 14 (see also GenBank Accession No. NP_077078.5). The term tryptase beta encompasses “full-length,” unprocessed tryptase beta as well as tryptase beta that results from post-translational modifications, including proteolytic processing. Full-length, pro-tryptase beta is thought to be processed in two proteolytic steps. First, autocatalytic intermolecular cleavage at R⁻³ occurs, particularly at acidic pH and in the presence of a polyanion (e.g., heparin or dextran sulfate). Next, the remaining pro′ dipeptide is removed (likely by dipeptidyl peptidase I). For full-length human tryptase beta 1, with reference to SEQ ID NO: 12 below, the underlined amino acid residues correspond to the native leader sequence, and the bolded amino acid residues correspond to the pro-domain, which are cleaved to form the mature protein (see, e.g., Sakai et al. J. Clin. Invest. 97:988-995, 1996)

(SEQ ID NO: 12)
MLNLLLLALPVLASRAYAAPAPGQALQRVGIVGGQEAPRSKWPWQVSLR
VHGPYWMHFCGGSLIHPQWVLTAAHCVGPDVKDLAALRVQLREQHLYYQ
DQLLPVSRIIVHPQFYTAQIGADIALLELEEPVNVSSHVHTVTLPPASE
TFPPGMPCWVTGWGDVDNDERLPPPFPLKQVKVPIMENHICDAKYHLGA
YTGDDVRIVRDDMLCAGNTRRDSCQGDSGGPLVCKVNGTWLQAGVVSWG
EGCAOPNRPGIYTRVTYYLDWIHHYVPKKP.

Mature, enzymatically active tryptase beta is typically a homotetramer or heterotetramer, although active monomer has been reported (see, e.g., Fukuoka et al. J. Immunol. 176:3165, 2006). The subunits of the tryptase beta tetramer are held together by hydrophobic and polar interactions between subunits and stabilized by polyanions (particularly heparin and dextran sulfate). The term tryptase can refer to tryptase tetramer or tryptase monomer. Exemplary sequences for mature human tryptase beta 1, beta 2, and beta 3 are shown in SEQ ID NO: 15, SEQ ID NO: 16, and SEQ ID NO: 17, respectively. The active site of each subunit faces into a central pore of the tetramer, which measures approximately 50 ×30 angstroms (see, e.g., Pereira et al. Nature 392:306-311, 1998). The size of the central pore typically restricts access of the active sites by inhibitors. Exemplary substrates of tryptase beta include, but are not limited to, PAR2, C3, fibrinogen, fibronectin, and kininogen.

A “disorder” or “disease” is any condition that would benefit from treatment with a method of the invention. This includes chronic and acute disorders or diseases including those pathological conditions which predispose the mammal to the disorder in question. Examples of disorders to be treated herein include asthma (e.g., severe asthma (e.g., severe asthma that remains uncontrolled despite standard-of-care therapy), allergic asthma, or atopic asthma (e.g., mild atopic asthma)).

The term “administering” means the administration of a composition to a patient (e.g., a patient having asthma). The compositions (e.g., anti-tryptase antibodies) utilized in the methods and uses described herein can be administered, for example, parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously (e.g., by a pump (e.g., by a patch pump), subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. Parenteral administration includes intramuscular, intravenous, intraarterial, intraperitoneal, or subcutaneous administration. The compositions utilized in the methods described herein can also be administered systemically or locally. The method of administration can vary depending on various factors (e.g., the compound or composition being administered and the severity of the condition, disease, or disorder being treated).

The terms “therapeutic agent” or “agent” refer to any agent that is used to treat a disease, e.g., asthma (e.g., severe asthma (e.g., severe asthma that remains uncontrolled despite standard-of-care therapy), allergic asthma, or atopic asthma). A therapeutic agent may be, for example, a polypeptide(s) (e.g., an antibody, an immunoadhesin, or a peptibody), an aptamer, a small molecule that can bind to a protein, or a nucleic acid molecule that can bind to a nucleic acid molecule encoding a target (e.g., siRNA), and the like.

The terms “anti-tryptase antibody,” an “antibody that binds to tryptase,” and “antibody that specifically binds tryptase” refer to an antibody that is capable of binding tryptase with sufficient affinity such that the antibody is useful as a diagnostic and/or therapeutic agent in targeting tryptase. In one aspect, the extent of binding of an anti-tryptase antibody to an unrelated, non-tryptase protein is less than about 10% of the binding of the antibody to tryptase as measured, e.g., by a radioimmunoassay (RIA). In certain aspects, an antibody that binds to tryptase has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹M to 10⁻¹³ M). In certain aspects, an anti-tryptase antibody binds to an epitope of tryptase that is conserved among tryptase from different species. Exemplary anti-tryptase antibodies are described herein, in U.S. patent application publication Ser. No. US 2018/0230233, and in International Patent Application Publication No. WO 2018/148585, each of which is incorporated herein by reference in its entirety.

A “mast cell” is a type of granulocyte immune cell. Mast cells are typically present in mucosal and epithelial tissues throughout the body. Mast cells contain cytoplasmic granules that store inflammatory mediators, including tryptase (particularly tryptase beta), histamine, heparin, and cytokines. Mast cells can be activated by antigen/IgE/FcϵRI cross-linking, which can result in degranulation and release of inflammatory mediators. A mast cell may be a mucosal mast cell or a connective tissue mast cell. See, e.g., Krystel-Whittemore et al. Front. Immunol. 6:620, 2015.

The terms “patient,” “subject,” and “individual,” as used interchangeably herein, refer to any single animal, more specifically a mammal (including such non-human animals as, for example, cats, dogs, horses, rabbits, cows, pigs, sheep, zoo animals, and non-human primates) for which treatment is desired. Even more specifically, the patient herein is a human.

The term “effective amount” refers to an amount of a drug or therapeutic agent (e.g., an anti-tryptase antibody) effective to treat a disease or disorder (e.g., asthma (e.g., severe asthma (e.g., severe asthma that remains uncontrolled despite standard-of-care therapy), allergic asthma, or atopic asthma)) in a subject or patient, such as a mammal, e.g., a human.

As used herein, “therapy” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. Those in need of treatment include can include those already with the disorder as well as those at risk to have the disorder or those in whom the disorder is to be prevented. A patient may be successfully “treated” for asthma if, for example, after receiving an asthma therapy, the patient shows observable and/or measurable reduction in or absence of one or more of the following: recurrent wheezing, coughing, trouble breathing, chest tightness, symptoms that occur or worsen at night, symptoms that are triggered by cold air, exercise or exposure to allergens.

A “response” of a patient or a patient's “responsiveness” to treatment or therapy, for example a therapy including an anti-tryptase antibody, refers to the clinical or therapeutic benefit imparted to a patient at risk for or having asthma from or as a result of the treatment. A skilled person will readily be in position to determine whether a patient is responsive. For example, a patient having asthma who is responsive to a therapy including an anti-tryptase antibody may show observable and/or measurable reduction in or absence of one or more asthma symptoms, for example, recurrent wheezing, coughing, trouble breathing, chest tightness, symptoms that occur or worsen at night, symptoms that are triggered by cold air, exercise or exposure to allergens. In some aspects, a response may be an improvement in lung function.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “affinity-matured” antibody is one with one or more alterations in one or more HVRs and/or framework regions which result in an improvement in the affinity of the antibody for antigen, compared to a parent antibody which does not possess those alteration(s). Preferred affinity-matured antibodies will have nanomolar or even picomolar affinities for the target antigen. Affinity-matured antibodies are produced by procedures known in the art. For example, Marks et al. Bio/Technology 10:779-783, 1992 describes affinity maturation by VH and VL domain shuffling. Random mutagenesis of HVR and/or framework residues is described by: Barbas et al. Proc. Natl. Acad. Sci. USA 91:3809-3813, 1994; Schier et al. Gene 169:147-155, 1995; Yelton et al. J. ImmunoL 155:1994-2004, 1995; Jackson et al. J. Immunol. 154(7):3310-3319, 1995; and Hawkins et al. J. Mol. Biol. 226:889-896, 1992.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more, 60% or more, 70% or more, 80% or more, or 90% or more. In some aspects, the set of amino acid residues contacted by the antibody may be completely overlapping or partially overlapping with the set of amino acid residues contacted by the reference antibody. In some aspects, an antibody that binds to the same epitope as a reference antibody blocks binding of the reference antibody to its antigen in a competition assay by 50% or more, 60% or more, 70% or more, 80% or more, or 90% or more, and conversely, the reference antibody blocks binding of the antibody to its antigen in a competition assay by 50% or more, 60% or more, 70% or more, 80% or more, or 90% or more. An exemplary competition assay is provided herein. “Antibody fragments” comprise a portion of an intact antibody, preferably the antigen binding or variable region of the intact antibody. Examples of antibody fragments include Fab, Fab′, F(ab′)_{2,} and Fv fragments; diabodies; linear antibodies (see U.S. Pat. No. 5,641,870, Example 2; Zapata et al. Protein Eng. 8(10):1057-1062, 1995); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, Md., 1991.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315, 1994.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al. Proc. Natl. Acad. Sci. USA 90:6444-6448, 1993.

A “blocking” antibody or an “antagonist” antibody is one which inhibits or reduces biological activity of the antigen it binds. Certain blocking antibodies or antagonist antibodies substantially or completely inhibit the biological activity of the antigen. For example, with respect to anti-tryptase antibodies, in some aspects, the activity may be a tryptase enzymatic activity, e.g., protease activity. In other instances, the activity may be tryptase-mediated stimulation of bronchial smooth muscle cell proliferation and/or collagen-based contraction. In other instances, the activity may be mast cell histamine release (e.g., IgE-triggered histamine release and/or tryptase-triggered histamine release). In some aspects, an antibody can inhibit a biological activity of the antigen it binds by at least about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG_{3,} IgG_{4,} IgA₁, IgA₂. The heavy chain constant domains that correspond to the different classes of immunoglobulins are called α, δ, ϵ, γ, and μ, respectively.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcyRIII only, whereas monocytes express FcyRI, FcyRII, and FcyRIII. FcR expression on hematopoietic cells is summarized in Table 3 on page 464 of Ravetch et al. Annu. Rev. Immunol. 9:457-492, 1991. To assess ADCC activity of a molecule of interest, an in vitro ADCC assay, such as that described in US Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA 95:652-656, 1998.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain (see review M. in Daëron, Annu. Rev. Immunol. 15:203-234, 1997). FcRs are reviewed, for example, in Ravetch et al. Annu. Rev. Immunol. 9:457-492, 1991; Capel et al. Immunomethods 4:25-34, 1994; and de Haas et al. J. Lab. Clin. Med. 126:330-41, 1995. Other FcRs, including those to be identified in the future, are encompassed by the term “FcR” herein. The term also includes the neonatal receptor, FcRn, which is responsible for the transfer of maternal IgGs to the fetus (see, e.g., Guyer et al. J. Immunol. 117:587, 1976; and Kim et al. J. Immunol. 24:249, 1994).

“Human effector cells” are leukocytes which express one or more FcRs and perform effector functions. Preferably, the cells express at least FcyRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (Cl q) to antibodies (of the appropriate subclass) which are bound to their cognate antigen. To assess complement activation, a CDC assay, e.g., as described in Gazzano-Santoro et al. J. Immunol. Methods 202:163, 1996, can be performed.

An “epitope” is the portion of the antigen to which the antibody selectively binds. For a polypeptide antigen, a linear epitope can be a peptide portion of about 4-15 (e.g., 4, 5, 6, 7, 8, 9, 10, 11, 12, amino acid residues. A non-linear, conformational epitope may comprise residues of a polypeptide sequence brought to close vicinity in the three-dimensional (3D) structure of the protein. In some aspects, the epitope comprises amino acids that are within 4 angstroms (Å) of any atom of an antibody. In certain aspects, the epitope comprises amino acids that are within 3.5 Å, 3 Å, 2.5 Å, or 2 Å of any atom of an antibody. The amino acid residues of an antibody that contact an antigen (i.e., paratope) can be determined, for example, by determining the crystal structure of the antibody in complex with the antigen or by performing hydrogen/deuterium exchange.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human and/or has been made using any of the techniques for making human antibodies. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda Md., vols. 1-3, 1991. In one aspect, for the VL, the subgroup is subgroup kappa III or kappa IV as in Kabat et al. supra. In one aspect, for the VH, the subgroup is subgroup III as in Kabat et al. supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some aspects, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the HVRs (e.g., CDRs) correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525, 1986; Riechmann et al. Nature 332:323-329, 1988; and Presta, Curr. Op. Struct. Biol. 2:593-596, 1992.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:

(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);

(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

An “immunoconjugate” is an antibody conjugated to one or more heterologous molecule(s), including but not limited to a cytotoxic agent.

The term “isolated” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some aspects, an antibody is purified to greater than 95% or 99% purity as determined by, for example, electrophoretic (e.g., sodium dodecyl sulfate polyacrylamide gel electrophoresis (SDS-PAGE), isoelectric focusing (IEF), capillary electrophoresis) or chromatographic (e.g., ion exchange or reverse phase HPLC) methods. For a review of methods for assessment of antibody purity, see, for example, Flatman et al. J. Chromatogr. B 848:79-87, 2007. In preferred aspects, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope on an antigen, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In certain aspects, the term “monoclonal antibody” encompasses bispecific antibodies.

The term “bivalent antibody” refers to an antibody that has two binding sites for the antigen. A bivalent antibody can be, without limitation, in the IgG format or in the F(ab′)₂ format.

The term “multispecific antibody” is used in the broadest sense and covers an antibody that binds to two or more determinants or epitopes on one antigen or two or more determinants or epitopes on more than one antigen. Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). In certain aspects, the multispecific antibody is a bispecific antibody. “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one aspect, the multispecific antibody binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to the ability to bind only one epitope.

A “naked antibody” refers to an antibody that is not conjugated to a heterologous moiety (e.g., a cytotoxic moiety) or radiolabel. The naked antibody may be present in a pharmaceutical composition.

With regard to the binding of an antibody to a target molecule, the term “binds” or “binding” or “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one aspect, the term “specific binding” refers to binding where a molecule binds to a particular polypeptide or epitope on a particular polypeptide without substantially binding to any other polypeptide or polypeptide epitope.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al. supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al. supra). The “EU numbering system” or “EU index” is generally used when referring to a residue in an immunoglobulin heavy chain constant region (e.g., the EU index reported in Kabat et al. supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “pharmaceutical formulation” and “pharmaceutical composition” are used interchangeably herein, and refer to a preparation which is in such form as to permit the biological activity of an active ingredient contained therein to be effective, and which contains no additional components which are unacceptably toxic to a subject to which the formulation would be administered. Such formulations are sterile.

A “sterile” pharmaceutical formulation is aseptic or free or essentially free from all living microorganisms and their spores.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “kit” is any manufacture (e.g., a package or container) comprising at least one reagent, for example, a medicament for treatment of asthma (e.g., an anti-tryptase antibody). The manufacture is preferably promoted, distributed, or sold as a unit for performing the methods of the present disclosure.

### III. Therapeutic Methods, Compositions for Use, and Uses of the Invention

The present invention features methods of treating a patient having asthma, compositions (e.g., anti-tryptase antibodies) for use in treating a patient having an asthma, and uses of an anti-tryptase antibody, e.g., in the manufacture or preparation of a medicament for treating a patient having asthma.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1) of the anti-tryptase antibody of from about 300 mg to about 3600 mg. The C1D1 may be administered, for example, intravenously (IV) or subcutaneously (SC). In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1) of the anti-tryptase antibody of from about 300 mg to about 3600 mg. In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1) of the anti-tryptase antibody of from about 300 mg to about 3600 mg. In some aspects, the C1D1 is administered IV. In other aspects, the C1D1 is administered SC (e.g., by a pump (e.g., by a patch pump).

For example, in one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1) of the anti-tryptase antibody of from about 25 mg to about 450 mg (e.g., about 300 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1) of the anti-tryptase antibody of from about 25 mg to about 450 mg (e.g., about 300 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1) of the anti-tryptase antibody of from about 25 mg to about 450 mg (e.g., about 300 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 25 mg to about 450 mg, about 25 mg to about 425 mg, about 25 mg to about 400 mg, about 25 mg to about 375 mg, about 25 mg to about 350 mg, about 25 mg to about 325 mg, about 25 mg to about 300 mg, about 25 mg to about 275 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 450 mg, about 50 mg to about 425 mg, about 50 mg to about 400 mg, about 50 mg to about 375 mg, about 50 mg to about 350 mg, about 50 mg to about 325 mg, about 50 mg to about 300 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 450 mg, about 75 mg to about 425 mg, about 75 mg to about 400 mg, about 75 mg to about 375 mg, about 75 mg to about 350 mg, about 75 mg to about 325 mg, about 75 mg to about 300 mg, about 75 mg to about 275 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 450 mg, about 100 mg to about 425 mg, about 100 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 450 mg, about 125 mg to about 425 mg, about 125 mg to about 400 mg, about 125 mg to about 375 mg, about 125 mg to about 350 mg, about 125 mg to about 325 mg, about 125 mg to about 300 mg, about 125 mg to about 275 mg, about 125 mg to about 250 mg, about 125 mg to about 225 mg, about 125 mg to about 200 mg, about 125 mg to about 175 mg, about 125 mg to about 150 mg, about 150 mg to about 450 mg, about 150 mg to about 425 mg, about 150 mg to about 400 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 175 mg to about 450 mg, about 175 mg to about 425 mg, about 175 mg to about 400 mg, about 175 mg to about 375 mg, about 175 mg to about 350 mg, about 175 mg to about 325 mg, about 175 mg to about 300 mg, about 175 mg to about 275 mg, about 175 mg to about 250 mg, about 175 mg to about 225 mg, about 175 mg to about 200 mg, about 200 mg to about 450 mg, about 200 mg to about 425 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 225 mg to about 450 mg, about 225 mg to about 425 mg, about 225 mg to about 400 mg, about 225 mg to about 375 mg, about 225 mg to about 350 mg, about 225 mg to about 325 mg, about 225 mg to about 300 mg, about 225 mg to about 275 mg, about 225 mg to about 250 mg, about 250 mg to about 450 mg, about 250 mg to about 425 mg, about 250 mg to about 400 mg, about 250 mg to about 375 mg, about 250 mg to about 350 mg, about 250 mg to about 325 mg, about 250 mg to about 300 mg, about 250 mg to about 275 mg, about 275 mg to about 450 mg, about 275 mg to about 425 mg, about 275 mg to about 400 mg, about 275 mg to about 375 mg, about 275 mg to about 350 mg, about 275 mg to about 325 mg, about 275 mg to about 300 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 450 mg, about 325 mg to about 425 mg, about 325 mg to about 400 mg, about 325 mg to about 375 mg, about 325 mg to about 350 mg, about 350 mg to about 450 mg, about 350 mg to about 425 mg, about 350 mg to about 400 mg, about 350 mg to about 375 mg, about 375 mg to about 450 mg, about 375 mg to about 425 mg, about 375 mg to about 400 mg, about 400 mg to about 450 mg, about 400 mg to about 425 mg, or about 425 mg to about 450 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of from about 300 mg to about 750 mg (e.g., about 450 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 300 mg to about 750 mg (e.g., about 450 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 300 mg to about 750 mg (e.g., about 450 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 300 mg to about 750 mg, about 300 mg to about 725 mg, about 300 mg to about 700 mg, about 300 mg to about 675 mg, about 300 mg to about 650 mg, about 300 mg to about 625 mg, about 300 mg to about 600 mg, about 300 mg to about 575 mg, about 300 mg to about 550 mg, about 300 mg to about 525 mg, about 300 mg to about 500 mg, about 300 mg to about 475 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 750 mg, about 325 mg to about 725 mg, about 325 mg to about 700 mg, about 325 mg to about 675 mg, about 325 mg to about 650 mg, about 325 mg to about 625 mg, about 325 mg to about 600 mg, about 325 mg to about 575 mg, about 325 mg to about 550 mg, about 325 mg to about 525 mg, about 325 mg to about 500 mg, about 325 mg to about 475 mg, about 325 mg to about 450 mg, about 325 mg to about 425 mg, about 325 mg to about 400 mg, about 325 mg to about 375 mg, about 325 mg to about 350 mg, about 350 mg to about 750 mg, about 350 mg to about 725 mg, about 350 mg to about 700 mg, about 350 mg to about 675 mg, about 350 mg to about 650 mg, about 350 mg to about 625 mg, about 350 mg to about 600 mg, about 350 mg to about 575 mg, about 350 mg to about 550 mg, about 350 mg to about 525 mg, about 350 mg to about 500 mg, about 350 mg to about 475 mg, about 350 mg to about 450 mg, about 350 mg to about 425 mg, about 350 mg to about 400 mg, about 350 mg to about 375 mg, about 375 mg to about 750 mg, about 375 mg to about 725 mg, about 375 mg to about 700 mg, about 375 mg to about 675 mg, about 375 mg to about 650 mg, about 375 mg to about 625 mg, about 375 mg to about 600 mg, about 375 mg to about 575 mg, about 375 mg to about 550 mg, about 375 mg to about 525 mg, about 375 mg to about 500 mg, about 375 mg to about 475 mg, about 375 mg to about 450 mg, about 375 mg to about 425 mg, about 375 mg to about 400 mg, about 400 mg to about 750 mg, about 400 mg to about 725 mg, about 400 mg to about 700 mg, about 400 mg to about 675 mg, about 400 mg to about 650 mg, about 400 mg to about 625 mg, about 400 mg to about 600 mg, about 400 mg to about 575 mg, about 400 mg to about 550 mg, about 400 mg to about 525 mg, about 400 mg to about 500 mg, about 400 mg to about 475 mg, about 400 mg to about 450 mg, about 400 mg to about 425 mg, about 425 mg to about 750 mg, about 425 mg to about 725 mg, about 425 mg to about 700 mg, about 425 mg to about 675 mg, about 425 mg to about 650 mg, about 425 mg to about 625 mg, about 425 mg to about 600 mg, about 425 mg to about 575 mg, about 425 mg to about 550 mg, about 425 mg to about 525 mg, about 425 mg to about 500 mg, about 425 mg to about 475 mg, about 425 mg to about 450 mg, about 450 mg to about 750 mg, about 450 mg to about 725 mg, about 450 mg to about 700 mg, about 450 mg to about 675 mg, about 450 mg to about 650 mg, about 450 mg to about 625 mg, about 450 mg to about 600 mg, about 450 mg to about 575 mg, about 450 mg to about 550 mg, about 450 mg to about 525 mg, about 450 mg to about 500 mg, about 450 mg to about 475 mg, about 475 mg to about 750 mg, about 475 mg to about 725 mg, about 475 mg to about 700 mg, about 475 mg to about 675 mg, about 475 mg to about 650 mg, about 475 mg to about 625 mg, about 475 mg to about 600 mg, about 475 mg to about 575 mg, about 475 mg to about 550 mg, about 475 mg to about 525 mg, about 475 mg to about 500 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 525 mg to about 750 mg, about 525 mg to about 725 mg, about 525 mg to about 700 mg, about 525 mg to about 675 mg, about 525 mg to about 650 mg, about 525 mg to about 625 mg, about 525 mg to about 600 mg, about 525 mg to about 575 mg, about 525 mg to about 550 mg, about 550 mg to about 750 mg, about 550 mg to about 725 mg, about 550 mg to about 700 mg, about 550 mg to about 675 mg, about 550 mg to about 650 mg, about 550 mg to about 625 mg, about 550 mg to about 600 mg, about 550 mg to about 575 mg, about 575 mg to about 750 mg, about 575 mg to about 725 mg, about 575 mg to about 700 mg, about 575 mg to about 675 mg, about 575 mg to about 650 mg, about 575 mg to about 625 mg, about 575 mg to about 600 mg, about 600 mg to about 750 mg, about 600 mg to about 725 mg, about 600 mg to about 700 mg, about 600 mg to about 675 mg, about 600 mg to about 650 mg, about 600 mg to about 625 mg, about 625 mg to about 750 mg, about 625 mg to about 725 mg, about 625 mg to about 700 mg, about 625 mg to about 675 mg, about 625 mg to about 650 mg, about 650 mg to about 750 mg, about 650 mg to about 725 mg, about 650 mg to about 700 mg, about 650 mg to about 675 mg, about 675 mg to about 750 mg, about 675 mg to about 725 mg, about 675 mg to about 700 mg, about 700 mg to about 750 mg, about 700 mg to about 725 mg, or about 725 mg to about 750 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of from about 450 mg to about 900 mg (e.g., about 750 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 450 mg to about 900 mg (e.g., about 750 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 450 mg to about 900 mg (e.g., about 750 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 450 mg to about 900 mg, about 450 mg to about 875 mg, about 450 mg to about 850 mg, about 450 mg to about 825 mg, about 450 mg to about 800 mg, about 450 mg to about 775 mg, about 450 mg to about 750 mg, about 450 mg to about 725 mg, about 450 mg to about 700 mg, about 450 mg to about 675 mg, about 450 mg to about 650 mg, about 450 mg to about 625 mg, about 450 mg to about 600 mg, about 450 mg to about 575 mg, about 450 mg to about 550 mg, about 450 mg to about 525 mg, about 450 mg to about 500 mg, about 450 mg to about 475 mg, about 475 mg to about 900 mg, about 475 mg to about 875 mg, about 475 mg to about 850 mg, about 475 mg to about 825 mg, about 475 mg to about 800 mg, about 475 mg to about 775 mg, about 475 mg to about 750 mg, about 475 mg to about 725 mg, about 475 mg to about 700 mg, about 475 mg to about 675 mg, about 475 mg to about 650 mg, about 475 mg to about 625 mg, about 475 mg to about 600 mg, about 475 mg to about 575 mg, about 475 mg to about 550 mg, about 475 mg to about 525 mg, about 475 mg to about 500 mg, about 500 mg to about 900 mg, about 500 mg to about 875 mg, about 500 mg to about 850 mg, about 500 mg to about 825 mg, about 500 mg to about 800 mg, about 500 mg to about 775 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 500 mg to about 900 mg, about 500 mg to about 875 mg, about 500 mg to about 850 mg, about 500 mg to about 825 mg, about 500 mg to about 800 mg, about 500 mg to about 775 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 525 mg to about 900 mg, about 525 mg to about 875 mg, about 525 mg to about 850 mg, about 525 mg to about 825 mg, about 525 mg to about 800 mg, about 525 mg to about 775 mg, about 525 mg to about 750 mg, about 525 mg to about 725 mg, about 525 mg to about 700 mg, about 525 mg to about 675 mg, about 525 mg to about 650 mg, about 525 mg to about 625 mg, about 525 mg to about 600 mg, about 525 mg to about 575 mg, about 525 mg to about 550 mg, about 550 mg to about 900 mg, about 550 mg to about 875 mg, about 550 mg to about 850 mg, about 550 mg to about 825 mg, about 550 mg to about 800 mg, about 550 mg to about 775 mg, about 550 mg to about 750 mg, about 550 mg to about 725 mg, about 550 mg to about 700 mg, about 550 mg to about 675 mg, about 550 mg to about 650 mg, about 550 mg to about 625 mg, about 550 mg to about 600 mg, about 550 mg to about 575 mg, about 575 mg to about 900 mg, about 575 mg to about 875 mg, about 575 mg to about 850 mg, about 575 mg to about 825 mg, about 575 mg to about 800 mg, about 575 mg to about 775 mg, about 575 mg to about 750 mg, about 575 mg to about 725 mg, about 575 mg to about 700 mg, about 575 mg to about 675 mg, about 575 mg to about 650 mg, about 575 mg to about 625 mg, about 575 mg to about 600 mg, about 600 mg to about 900 mg, about 600 mg to about 875 mg, about 600 mg to about 850 mg, about 600 mg to about 825 mg, about 600 mg to about 800 mg, about 600 mg to about 775 mg, about 600 mg to about 750 mg, about 600 mg to about 725 mg, about 600 mg to about 700 mg, about 600 mg to about 675 mg, about 600 mg to about 650 mg, about 600 mg to about 625 mg, about 625 mg to about 900 mg, about 625 mg to about 875 mg, about 625 mg to about 850 mg, about 625 mg to about 825 mg, about 625 mg to about 800 mg, about 625 mg to about 775 mg, about 625 mg to about 750 mg, about 625 mg to about 725 mg, about 625 mg to about 700 mg, about 625 mg to about 675 mg, about 625 mg to about 650 mg, about 650 mg to about 900 mg, about 650 mg to about 875 mg, about 650 mg to about 850 mg, about 650 mg to about 825 mg, about 650 mg to about 800 mg, about 650 mg to about 775 mg, about 650 mg to about 750 mg, about 650 mg to about 725 mg, about 650 mg to about 700 mg, about 650 mg to about 675 mg, about 675 mg to about 900 mg, about 675 mg to about 875 mg, about 675 mg to about 850 mg, about 675 mg to about 825 mg, about 675 mg to about 800 mg, about 675 mg to about 775 mg, about 675 mg to about 750 mg, about 675 mg to about 725 mg, about 675 mg to about 700 mg, about 700 mg to about 900 mg, about 700 mg to about 875 mg, about 700 mg to about 850 mg, about 700 mg to about 825 mg, about 700 mg to about 800 mg, about 700 mg to about 775 mg, about 700 mg to about 750 mg, about 700 mg to about 725 mg, about 725 mg to about 900 mg, about 725 mg to about 875 mg, about 725 mg to about 850 mg, about 725 mg to about 825 mg, about 725 mg to about 800 mg, about 725 mg to about 775 mg, about 725 mg to about 750 mg, about 750 mg to about 900 mg, about 750 mg to about 875 mg, about 750 mg to about 850 mg, about 750 mg to about 825 mg, about 750 mg to about 800 mg, about 750 mg to about 775 mg, about 775 mg to about 900 mg, about 775 mg to about 875 mg, about 775 mg to about 850 mg, about 775 mg to about 825 mg, about 775 mg to about 800 mg, about 800 mg to about 900 mg, about 800 mg to about 875 mg, about 800 mg to about 850 mg, about 800 mg to about 825 mg, about 825 mg to about 900 mg, about 825 mg to about 875 mg, about 825 mg to about 850 mg, about 850 mg to about 900 mg, about 850 mg to about 875 mg, or about 875 mg to about 900 mg.

In one aspect, provided herein is a method of treating a patient having asthma, the method including administering to a patient having asthma an anti-tryptase antibody, wherein the anti-tryptase antibody is for administration to a patient having asthma in a dosing regimen including a dosing cycle, wherein the dosing cycle includes a first dose (C1D1 ) of the anti-tryptase antibody of from about 750 mg to about 1350 mg (e.g., about 900 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 750 mg to about 1350 mg (e.g., about 900 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 750 mg to about 1350 mg (e.g., about 900 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 750 mg to about 1350 mg, about 750 mg to about 1325 mg, about 750 mg to about 1300 mg, about 750 mg to about 1275 mg, about 750 mg to about 1250 mg, about 750 mg to about 1225 mg, about 750 mg to about 1200 mg, about 750 mg to about 1175 mg, about 750 mg to about 1150 mg, about 750 mg to about 1125 mg, about 750 mg to about 1100 mg, about 750 mg to about 1075 mg, about 750 mg to about 1050 mg, about 750 mg to about 1025 mg, about 750 mg to about 1000 mg, about 750 mg to about 975 mg, about 750 mg to about 950 mg, about 750 mg to about 925 mg, about 750 mg to about 900 mg, about 750 mg to about 875 mg, about 750 mg to about 850 mg, about 750 mg to about 825 mg, about 750 mg to about 800 mg, about 750 mg to about 775 mg, about 775 mg to about 1350 mg, about 775 mg to about 1325 mg, about 775 mg to about 1300 mg, about 775 mg to about 1275 mg, about 775 mg to about 1250 mg, about 775 mg to about 1225 mg, about 775 mg to about 1200 mg, about 775 mg to about 1175 mg, about 775 mg to about 1150 mg, about 775 mg to about 1125 mg, about 775 mg to about 1100 mg, about 775 mg to about 1075 mg, about 775 mg to about 1050 mg, about 775 mg to about 1025 mg, about 775 mg to about 1000 mg, about 775 mg to about 975 mg, about 775 mg to about 950 mg, about 775 mg to about 925 mg, about 775 mg to about 900 mg, about 775 mg to about 875 mg, about 775 mg to about 850 mg, about 775 mg to about 825 mg, about 775 mg to about 800 mg, about 800 mg to about 1350 mg, about 800 mg to about 1325 mg, about 800 mg to about 1300 mg, about 800 mg to about 1275 mg, about 800 mg to about 1250 mg, about 800 mg to about 1225 mg, about 800 mg to about 1200 mg, about 800 mg to about 1175 mg, about 800 mg to about 1150 mg, about 800 mg to about 1125 mg, about 800 mg to about 1100 mg, about 800 mg to about 1075 mg, about 800 mg to about 1050 mg, about 800 mg to about 1025 mg, about 800 mg to about 1000 mg, about 800 mg to about 975 mg, about 800 mg to about 950 mg, about 800 mg to about 925 mg, about 800 mg to about 900 mg, about 800 mg to about 875 mg, about 800 mg to about 850 mg, about 800 mg to about 825 mg, about 825 mg to about 1350 mg, about 825 mg to about 1325 mg, about 825 mg to about 1300 mg, about 825 mg to about 1275 mg, about 825 mg to about 1250 mg, about 825 mg to about 1225 mg, about 825 mg to about 1200 mg, about 825 mg to about 1175 mg, about 825 mg to about 1150 mg, about 825 mg to about 1125 mg, about 825 mg to about 1100 mg, about 825 mg to about 1075 mg, about 825 mg to about 1050 mg, about 825 mg to about 1025 mg, about 825 mg to about 1000 mg, about 825 mg to about 975 mg, about 825 mg to about 950 mg, about 825 mg to about 925 mg, about 825 mg to about 900 mg, about 825 mg to about 875 mg, about 825 mg to about 850 mg, about 850 mg to about 1350 mg, about 850 mg to about 1325 mg, about 850 mg to about 1300 mg, about 850 mg to about 1275 mg, about 850 mg to about 1250 mg, about 850 mg to about 1225 mg, about 850 mg to about 1200 mg, about 850 mg to about 1175 mg, about 850 mg to about 1150 mg, about 850 mg to about 1125 mg, about 850 mg to about 1100 mg, about 850 mg to about 1075 mg, about 850 mg to about 1050 mg, about 850 mg to about 1025 mg, about 850 mg to about 1000 mg, about 850 mg to about 975 mg, about 850 mg to about 950 mg, about 850 mg to about 925 mg, about 850 mg to about 900 mg, about 850 mg to about 875 mg, about 875 mg to about 1350 mg, about 875 mg to about 1325 mg, about 875 mg to about 1300 mg, about 875 mg to about 1275 mg, about 875 mg to about 1250 mg, about 875 mg to about 1225 mg, about 875 mg to about 1200 mg, about 875 mg to about 1175 mg, about 875 mg to about 1150 mg, about 875 mg to about 1125 mg, about 875 mg to about 1100 mg, about 875 mg to about 1075 mg, about 875 mg to about 1050 mg, about 875 mg to about 1025 mg, about 875 mg to about 1000 mg, about 875 mg to about 975 mg, about 875 mg to about 950 mg, about 875 mg to about 925 mg, about 875 mg to about 900 mg, about 900 mg to about 1350 mg, about 900 mg to about 1325 mg, about 900 mg to about 1300 mg, about 900 mg to about 1275 mg, about 900 mg to about 1250 mg, about 900 mg to about 1225 mg, about 900 mg to about 1200 mg, about 900 mg to about 1175 mg, about 900 mg to about 1150 mg, about 900 mg to about 1125 mg, about 900 mg to about 1100 mg, about 900 mg to about 1075 mg, about 900 mg to about 1050 mg, about 900 mg to about 1025 mg, about 900 mg to about 1000 mg, about 900 mg to about 975 mg, about 900 mg to about 950 mg, about 900 mg to about 925 mg, about 925 mg to about 1350 mg, about 925 mg to about 1325 mg, about 925 mg to about 1300 mg, about 925 mg to about 1275 mg, about 925 mg to about 1250 mg, about 925 mg to about 1225 mg, about 925 mg to about 1200 mg, about 925 mg to about 1175 mg, about 925 mg to about 1150 mg, about 925 mg to about 1125 mg, about 925 mg to about 1100 mg, about 925 mg to about 1075 mg, about 925 mg to about 1050 mg, about 925 mg to about 1025 mg, about 925 mg to about 1000 mg, about 925 mg to about 975 mg, about 925 mg to about 950 mg, about 950 mg to about 1350 mg, about 950 mg to about 1325 mg, about 950 mg to about 1300 mg, about 950 mg to about 1275 mg, about 950 mg to about 1250 mg, about 950 mg to about 1225 mg, about 950 mg to about 1200 mg, about 950 mg to about 1175 mg, about 950 mg to about 1150 mg, about 950 mg to about 1125 mg, about 950 mg to about 1100 mg, about 950 mg to about 1075 mg, about 950 mg to about 1050 mg, about 950 mg to about 1025 mg, about 950 mg to about 1000 mg, about 950 mg to about 975 mg, about 975 mg to about 1350 mg, about 975 mg to about 1325 mg, about 975 mg to about 1300 mg, about 975 mg to about 1275 mg, about 975 mg to about 1250 mg, about 975 mg to about 1225 mg, about 975 mg to about 1200 mg, about 975 mg to about 1175 mg, about 975 mg to about 1150 mg, about 975 mg to about 1125 mg, about 975 mg to about 1100 mg, about 975 mg to about 1075 mg, about 975 mg to about 1050 mg, about 975 mg to about 1025 mg, about 975 mg to about 1000 mg, about 1000 mg to about 1350 mg, about 1000 mg to about 1325 mg, about 1000 mg to about 1300 mg, about 1000 mg to about 1275 mg, about 1000 mg to about 1250 mg, about 1000 mg to about 1225 mg, about 1000 mg to about 1200 mg, about 1000 mg to about 1175 mg, about 1000 mg to about 1150 mg, about 1000 mg to about 1125 mg, about 1000 mg to about 1100 mg, about 1000 mg to about 1075 mg, about 1000 mg to about 1050 mg, about 1000 mg to about 1025 mg, about 1025 mg to about 1350 mg, about 1025 mg to about 1325 mg, about 1025 mg to about 1300 mg, about 1025 mg to about 1275 mg, about 1025 mg to about 1250 mg, about 1025 mg to about 1225 mg, about 1025 mg to about 1200 mg, about 1025 mg to about 1175 mg, about 1025 mg to about 1150 mg, about 1025 mg to about 1125 mg, about 1025 mg to about 1100 mg, about 1025 mg to about 1075 mg, about 1025 mg to about 1050 mg, about 1050 mg to about 1350 mg, about 1050 mg to about 1325 mg, about 1050 mg to about 1300 mg, about 1050 mg to about 1275 mg, about 1050 mg to about 1250 mg, about 1050 mg to about 1225 mg, about 1050 mg to about 1200 mg, about 1050 mg to about 1175 mg, about 1050 mg to about 1150 mg, about 1050 mg to about 1125 mg, about 1050 mg to about 1100 mg, about 1050 mg to about 1075 mg, about 1075 mg to about 1350 mg, about 1075 mg to about 1325 mg, about 1075 mg to about 1300 mg, about 1075 mg to about 1275 mg, about 1075 mg to about 1250 mg, about 1075 mg to about 1225 mg, about 1075 mg to about 1200 mg, about 1075 mg to about 1175 mg, about 1075 mg to about 1150 mg, about 1075 mg to about 1125 mg, about 1075 mg to about 1100 mg, about 1100 mg to about 1350 mg, about 1100 mg to about 1325 mg, about 1100 mg to about 1300 mg, about 1100 mg to about 1275 mg, about 1100 mg to about 1250 mg, about 1100 mg to about 1225 mg, about 1100 mg to about 1200 mg, about 1100 mg to about 1175 mg, about 1100 mg to about 1150 mg, about 1100 mg to about 1125 mg, about 1125 mg to about 1350 mg, about 1125 mg to about 1325 mg, about 1125 mg to about 1300 mg, about 1125 mg to about 1275 mg, about 1125 mg to about 1250 mg, about 1125 mg to about 1225 mg, about 1125 mg to about 1200 mg, about 1125 mg to about 1175 mg, about 1125 mg to about 1150 mg, about 1150 mg to about 1350 mg, about 1150 mg to about 1325 mg, about 1150 mg to about 1300 mg, about 1150 mg to about 1275 mg, about 1150 mg to about 1250 mg, about 1150 mg to about 1225 mg, about 1150 mg to about 1200 mg, about 1150 mg to about 1175 mg, about 1175 mg to about 1350 mg, about 1175 mg to about 1325 mg, about 1175 mg to about 1300 mg, about 1175 mg to about 1275 mg, about 1175 mg to about 1250 mg, about 1175 mg to about 1225 mg, about 1175 mg to about 1200 mg, about 1200 mg to about 1350 mg, about 1200 mg to about 1325 mg, about 1200 mg to about 1300 mg, about 1200 mg to about 1275 mg, about 1200 mg to about 1250 mg, about 1200 mg to about 1225 mg, about 1225 mg to about 1350 mg, about 1225 mg to about 1325 mg, about 1225 mg to about 1300 mg, about 1225 mg to about 1275 mg, about 1225 mg to about 1250 mg, about 1250 mg to about 1350 mg, about 1250 mg to about 1325 mg, about 1250 mg to about 1300 mg, about 1250 mg to about 1275 mg, about 1275 mg to about 1350 mg, about 1275 mg to about 1325 mg, about 1275 mg to about 1300 mg, about 1300 mg to about 1350 mg, about 1300 mg to about 1325 mg, or about 1325 mg to about 1350 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of from about 900 mg to about 1800 mg (e.g., about 1350 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 900 mg to about 1800 mg (e.g., about 1350 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 900 mg to about 1800 mg (e.g., about 1350 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 900 mg to about 1800 mg, about 900 mg to about 1775 mg, about 900 mg to about 1750 mg, about 900 mg to about 1725 mg, about 900 mg to about 1700 mg, about 900 mg to about 1675 mg, about 900 mg to about 1650 mg, about 900 mg to about 1625 mg, about 900 mg to about 1600 mg, about 900 mg to about 1575 mg, about 900 mg to about 1550 mg, about 900 mg to about 1525 mg, about 900 mg to about 1500 mg, about 900 mg to about 1475 mg, about 900 mg to about 1450 mg, about 900 mg to about 1425 mg, about 900 mg to about 1400 mg, about 900 mg to about 1375 mg, about 900 mg to about 1350 mg, about 900 mg to about 1325 mg, about 900 mg to about 1300 mg, about 900 mg to about 1275 mg, about 900 mg to about 1250 mg, about 900 mg to about 1225 mg, about 900 mg to about 1200 mg, about 900 mg to about 1175 mg, about 900 mg to about 1150 mg, about 900 mg to about 1125 mg, about 900 mg to about 1100 mg, about 900 mg to about 1075 mg, about 900 mg to about 1050 mg, about 900 mg to about 1025 mg, about 900 mg to about 1000 mg, about 900 mg to about 975 mg, about 900 mg to about 950 mg, about 900 mg to about 925 mg, about 925 mg to about 1800 mg, about 925 mg to about 1775 mg, about 925 mg to about 1750 mg, about 925 mg to about 1725 mg, about 925 mg to about 1700 mg, about 925 mg to about 1675 mg, about 925 mg to about 1650 mg, about 925 mg to about 1625 mg, about 925 mg to about 1600 mg, about 925 mg to about 1575 mg, about 925 mg to about 1550 mg, about 925 mg to about 1525 mg, about 925 mg to about 1500 mg, about 925 mg to about 1475 mg, about 925 mg to about 1450 mg, about 925 mg to about 1425 mg, about 925 mg to about 1400 mg, about 925 mg to about 1375 mg, about 925 mg to about 1350 mg, about 925 mg to about 1325 mg, about 925 mg to about 1300 mg, about 925 mg to about 1275 mg, about 925 mg to about 1250 mg, about 925 mg to about 1225 mg, about 925 mg to about 1200 mg, about 925 mg to about 1175 mg, about 925 mg to about 1150 mg, about 925 mg to about 1125 mg, about 925 mg to about 1100 mg, about 925 mg to about 1075 mg, about 925 mg to about 1050 mg, about 925 mg to about 1025 mg, about 925 mg to about 1000 mg, about 925 mg to about 975 mg, about 925 mg to about 950 mg, about 950 mg to about 1800 mg, about 950 mg to about 1775 mg, about 950 mg to about 1750 mg, about 950 mg to about 1725 mg, about 950 mg to about 1700 mg, about 950 mg to about 1675 mg, about 950 mg to about 1650 mg, about 950 mg to about 1625 mg, about 950 mg to about 1600 mg, about 950 mg to about 1575 mg, about 950 mg to about 1550 mg, about 950 mg to about 1525 mg, about 950 mg to about 1500 mg, about 950 mg to about 1475 mg, about 950 mg to about 1450 mg, about 950 mg to about 1425 mg, about 950 mg to about 1400 mg, about 950 mg to about 1375 mg, about 950 mg to about 1350 mg, about 950 mg to about 1325 mg, about 950 mg to about 1300 mg, about 950 mg to about 1275 mg, about 950 mg to about 1250 mg, about 950 mg to about 1225 mg, about 950 mg to about 1200 mg, about 950 mg to about 1175 mg, about 950 mg to about 1150 mg, about 950 mg to about 1125 mg, about 950 mg to about 1100 mg, about 950 mg to about 1075 mg, about 950 mg to about 1050 mg, about 950 mg to about 1025 mg, about 950 mg to about 1000 mg, about 950 mg to about 975 mg, about 975 mg to about 1800 mg, about 975 mg to about 1775 mg, about 975 mg to about 1750 mg, about 975 mg to about 1725 mg, about 975 mg to about 1700 mg, about 975 mg to about 1675 mg, about 975 mg to about 1650 mg, about 975 mg to about 1625 mg, about 975 mg to about 1600 mg, about 975 mg to about 1575 mg, about 975 mg to about 1550 mg, about 975 mg to about 1525 mg, about 975 mg to about 1500 mg, about 975 mg to about 1475 mg, about 975 mg to about 1450 mg, about 975 mg to about 1425 mg, about 975 mg to about 1400 mg, about 975 mg to about 1375 mg, about 975 mg to about 1350 mg, about 975 mg to about 1325 mg, about 975 mg to about 1300 mg, about 975 mg to about 1275 mg, about 975 mg to about 1250 mg, about 975 mg to about 1225 mg, about 975 mg to about 1200 mg, about 975 mg to about 1175 mg, about 975 mg to about 1150 mg, about 975 mg to about 1125 mg, about 975 mg to about 1100 mg, about 975 mg to about 1075 mg, about 975 mg to about 1050 mg, about 975 mg to about 1025 mg, about 975 mg to about 1000 mg, about 1000 mg to about 1800 mg, about 1000 mg to about 1775 mg, about 1000 mg to about 1750 mg, about 1000 mg to about 1725 mg, about 1000 mg to about 1700 mg, about 1000 mg to about 1675 mg, about 1000 mg to about 1650 mg, about 1000 mg to about 1625 mg, about 1000 mg to about 1600 mg, about 1000 mg to about 1575 mg, about 1000 mg to about 1550 mg, about 1000 mg to about 1525 mg, about 1000 mg to about 1500 mg, about 1000 mg to about 1475 mg, about 1000 mg to about 1450 mg, about 1000 mg to about 1425 mg, about 1000 mg to about 1400 mg, about 1000 mg to about 1375 mg, about 1000 mg to about 1350 mg, about 1000 mg to about 1325 mg, about 1000 mg to about 1300 mg, about 1000 mg to about 1275 mg, about 1000 mg to about 1250 mg, about 1000 mg to about 1225 mg, about 1000 mg to about 1200 mg, about 1000 mg to about 1175 mg, about 1000 mg to about 1150 mg, about 1000 mg to about 1125 mg, about 1000 mg to about 1100 mg, about 1000 mg to about 1075 mg, about 1000 mg to about 1050 mg, about 1000 mg to about 1025 mg, about 1025 mg to about 1800 mg, about 1025 mg to about 1775 mg, about 1025 mg to about 1750 mg, about 1025 mg to about 1725 mg, about 1025 mg to about 1700 mg, about 1025 mg to about 1675 mg, about 1025 mg to about 1650 mg, about 1025 mg to about 1625 mg, about 1025 mg to about 1600 mg, about 1025 mg to about 1575 mg, about 1025 mg to about 1550 mg, about 1025 mg to about 1525 mg, about 1025 mg to about 1500 mg, about 1025 mg to about 1475 mg, about 1025 mg to about 1450 mg, about 1025 mg to about 1425 mg, about 1025 mg to about 1400 mg, about 1025 mg to about 1375 mg, about 1025 mg to about 1350 mg, about 1025 mg to about 1325 mg, about 1025 mg to about 1300 mg, about 1025 mg to about 1275 mg, about 1025 mg to about 1250 mg, about 1025 mg to about 1225 mg, about 1025 mg to about 1200 mg, about 1025 mg to about 1175 mg, about 1025 mg to about 1150 mg, about 1025 mg to about 1125 mg, about 1025 mg to about 1100 mg, about 1025 mg to about 1075 mg, about 1025 mg to about 1050 mg, about 1050 mg to about 1800 mg, about 1050 mg to about 1775 mg, about 1050 mg to about 1750 mg, about 1050 mg to about 1725 mg, about 1050 mg to about 1700 mg, about 1050 mg to about 1675 mg, about 1050 mg to about 1650 mg, about 1050 mg to about 1625 mg, about 1050 mg to about 1600 mg, about 1050 mg to about 1575 mg, about 1050 mg to about 1550 mg, about 1050 mg to about 1525 mg, about 1050 mg to about 1500 mg, about 1050 mg to about 1475 mg, about 1050 mg to about 1450 mg, about 1050 mg to about 1425 mg, about 1050 mg to about 1400 mg, about 1050 mg to about 1375 mg, about 1050 mg to about 1350 mg, about 1050 mg to about 1325 mg, about 1050 mg to about 1300 mg, about 1050 mg to about 1275 mg, about 1050 mg to about 1250 mg, about 1050 mg to about 1225 mg, about 1050 mg to about 1200 mg, about 1050 mg to about 1175 mg, about 1050 mg to about 1150 mg, about 1050 mg to about 1125 mg, about 1050 mg to about 1100 mg, about 1050 mg to about 1075 mg, about 1075 mg to about 1800 mg, about 1075 mg to about 1775 mg, about 1075 mg to about 1750 mg, about 1075 mg to about 1725 mg, about 1075 mg to about 1700 mg, about 1075 mg to about 1675 mg, about 1075 mg to about 1650 mg, about 1075 mg to about 1625 mg, about 1075 mg to about 1600 mg, about 1075 mg to about 1575 mg, about 1075 mg to about 1550 mg, about 1075 mg to about 1525 mg, about 1075 mg to about 1500 mg, about 1075 mg to about 1475 mg, about 1075 mg to about 1450 mg, about 1075 mg to about 1425 mg, about 1075 mg to about 1400 mg, about 1075 mg to about 1375 mg, about 1075 mg to about 1350 mg, about 1075 mg to about 1325 mg, about 1075 mg to about 1300 mg, about 1075 mg to about 1275 mg, about 1075 mg to about 1250 mg, about 1075 mg to about 1225 mg, about 1075 mg to about 1200 mg, about 1075 mg to about 1175 mg, about 1075 mg to about 1150 mg, about 1075 mg to about 1125 mg, about 1075 mg to about 1100 mg, about 1100 mg to about 1800 mg, about 1100 mg to about 1775 mg, about 1100 mg to about 1750 mg, about 1100 mg to about 1725 mg, about 1100 mg to about 1700 mg, about 1100 mg to about 1675 mg, about 1100 mg to about 1650 mg, about 1100 mg to about 1625 mg, about 1100 mg to about 1600 mg, about 1100 mg to about 1575 mg, about 1100 mg to about 1550 mg, about 1100 mg to about 1525 mg, about 1100 mg to about 1500 mg, about 1100 mg to about 1475 mg, about 1100 mg to about 1450 mg, about 1100 mg to about 1425 mg, about 1100 mg to about 1400 mg, about 1100 mg to about 1375 mg, about 1100 mg to about 1350 mg, about 1100 mg to about 1325 mg, about 1100 mg to about 1300 mg, about 1100 mg to about 1275 mg, about 1100 mg to about 1250 mg, about 1100 mg to about 1225 mg, about 1100 mg to about 1200 mg, about 1100 mg to about 1175 mg, about 1100 mg to about 1150 mg, about 1100 mg to about 1125 mg, about 1125 mg to about 1800 mg, about 1125 mg to about 1775 mg, about 1125 mg to about 1750 mg, about 1125 mg to about 1725 mg, about 1125 mg to about 1700 mg, about 1125 mg to about 1675 mg, about 1125 mg to about 1650 mg, about 1125 mg to about 1625 mg, about 1125 mg to about 1600 mg, about 1125 mg to about 1575 mg, about 1125 mg to about 1550 mg, about 1125 mg to about 1525 mg, about 1125 mg to about 1500 mg, about 1125 mg to about 1475 mg, about 1125 mg to about 1450 mg, about 1125 mg to about 1425 mg, about 1125 mg to about 1400 mg, about 1125 mg to about 1375 mg, about 1125 mg to about 1350 mg, about 1125 mg to about 1325 mg, about 1125 mg to about 1300 mg, about 1125 mg to about 1275 mg, about 1125 mg to about 1250 mg, about 1125 mg to about 1225 mg, about 1125 mg to about 1200 mg, about 1125 mg to about 1175 mg, about 1125 mg to about 1150 mg, about 1150 mg to about 1800 mg, about 1150 mg to about 1775 mg, about 1150 mg to about 1750 mg, about 1150 mg to about 1725 mg, about 1150 mg to about 1700 mg, about 1150 mg to about 1675 mg, about 1150 mg to about 1650 mg, about 1150 mg to about 1625 mg, about 1150 mg to about 1600 mg, about 1150 mg to about 1575 mg, about 1150 mg to about 1550 mg, about 1150 mg to about 1525 mg, about 1150 mg to about 1500 mg, about 1150 mg to about 1475 mg, about 1150 mg to about 1450 mg, about 1150 mg to about 1425 mg, about 1150 mg to about 1400 mg, about 1150 mg to about 1375 mg, about 1150 mg to about 1350 mg, about 1150 mg to about 1325 mg, about 1150 mg to about 1300 mg, about 1150 mg to about 1275 mg, about 1150 mg to about 1250 mg, about 1150 mg to about 1225 mg, about 1150 mg to about 1200 mg, about 1150 mg to about 1175 mg, about 1175 mg to about 1800 mg, about 1175 mg to about 1775 mg, about 1175 mg to about 1750 mg, about 1175 mg to about 1725 mg, about 1175 mg to about 1700 mg, about 1175 mg to about 1675 mg, about 1175 mg to about 1650 mg, about 1175 mg to about 1625 mg, about 1175 mg to about 1600 mg, about 1175 mg to about 1575 mg, about 1175 mg to about 1550 mg, about 1175 mg to about 1525 mg, about 1175 mg to about 1500 mg, about 1175 mg to about 1475 mg, about 1175 mg to about 1450 mg, about 1175 mg to about 1425 mg, about 1175 mg to about 1400 mg, about 1175 mg to about 1375 mg, about 1175 mg to about 1350 mg, about 1175 mg to about 1325 mg, about 1175 mg to about 1300 mg, about 1175 mg to about 1275 mg, about 1175 mg to about 1250 mg, about 1175 mg to about 1225 mg, about 1175 mg to about 1200 mg, about 1200 mg to about 1800 mg, about 1200 mg to about 1775 mg, about 1200 mg to about 1750 mg, about 1200 mg to about 1725 mg, about 1200 mg to about 1700 mg, about 1200 mg to about 1675 mg, about 1200 mg to about 1650 mg, about 1200 mg to about 1625 mg, about 1200 mg to about 1600 mg, about 1200 mg to about 1575 mg, about 1200 mg to about 1550 mg, about 1200 mg to about 1525 mg, about 1200 mg to about 1500 mg, about 1200 mg to about 1475 mg, about 1200 mg to about 1450 mg, about 1200 mg to about 1425 mg, about 1200 mg to about 1400 mg, about 1200 mg to about 1375 mg, about 1200 mg to about 1350 mg, about 1200 mg to about 1325 mg, about 1200 mg to about 1300 mg, about 1200 mg to about 1275 mg, about 1200 mg to about 1250 mg, about 1200 mg to about 1225 mg, about 1225 mg to about 1800 mg, about 1225 mg to about 1775 mg, about 1225 mg to about 1750 mg, about 1225 mg to about 1725 mg, about 1225 mg to about 1700 mg, about 1225 mg to about 1675 mg, about 1225 mg to about 1650 mg, about 1225 mg to about 1625 mg, about 1225 mg to about 1600 mg, about 1225 mg to about 1575 mg, about 1225 mg to about 1550 mg, about 1225 mg to about 1525 mg, about 1225 mg to about 1500 mg, about 1225 mg to about 1475 mg, about 1225 mg to about 1450 mg, about 1225 mg to about 1425 mg, about 1225 mg to about 1400 mg, about 1225 mg to about 1375 mg, about 1225 mg to about 1350 mg, about 1225 mg to about 1325 mg, about 1225 mg to about 1300 mg, about 1225 mg to about 1275 mg, about 1225 mg to about 1250 mg, about 1250 mg to about 1800 mg, about 1250 mg to about 1775 mg, about 1250 mg to about 1750 mg, about 1250 mg to about 1725 mg, about 1250 mg to about 1700 mg, about 1250 mg to about 1675 mg, about 1250 mg to about 1650 mg, about 1250 mg to about 1625 mg, about 1250 mg to about 1600 mg, about 1250 mg to about 1575 mg, about 1250 mg to about 1550 mg, about 1250 mg to about 1525 mg, about 1250 mg to about 1500 mg, about 1250 mg to about 1475 mg, about 1250 mg to about 1450 mg, about 1250 mg to about 1425 mg, about 1250 mg to about 1400 mg, about 1250 mg to about 1375 mg, about 1250 mg to about 1350 mg, about 1250 mg to about 1325 mg, about 1250 mg to about 1300 mg, about 1250 mg to about 1275 mg, about 1275 mg to about 1800 mg, about 1275 mg to about 1775 mg, about 1275 mg to about 1750 mg, about 1275 mg to about 1725 mg, about 1275 mg to about 1700 mg, about 1275 mg to about 1675 mg, about 1275 mg to about 1650 mg, about 1275 mg to about 1625 mg, about 1275 mg to about 1600 mg, about 1275 mg to about 1575 mg, about 1275 mg to about 1550 mg, about 1275 mg to about 1525 mg, about 1275 mg to about 1500 mg, about 1275 mg to about 1475 mg, about 1275 mg to about 1450 mg, about 1275 mg to about 1425 mg, about 1275 mg to about 1400 mg, about 1275 mg to about 1375 mg, about 1275 mg to about 1350 mg, about 1275 mg to about 1325 mg, about 1275 mg to about 1300 mg, about 1300 mg to about 1800 mg, about 1300 mg to about 1775 mg, about 1300 mg to about 1750 mg, about 1300 mg to about 1725 mg, about 1300 mg to about 1700 mg, about 1300 mg to about 1675 mg, about 1300 mg to about 1650 mg, about 1300 mg to about 1625 mg, about 1300 mg to about 1600 mg, about 1300 mg to about 1575 mg, about 1300 mg to about 1550 mg, about 1300 mg to about 1525 mg, about 1300 mg to about 1500 mg, about 1300 mg to about 1475 mg, about 1300 mg to about 1450 mg, about 1300 mg to about 1425 mg, about 1300 mg to about 1400 mg, about 1300 mg to about 1375 mg, about 1300 mg to about 1350 mg, about 1300 mg to about 1325 mg, about 1325 mg to about 1800 mg, about 1325 mg to about 1775 mg, about 1325 mg to about 1750 mg, about 1325 mg to about 1725 mg, about 1325 mg to about 1700 mg, about 1325 mg to about 1675 mg, about 1325 mg to about 1650 mg, about 1325 mg to about 1625 mg, about 1325 mg to about 1600 mg, about 1325 mg to about 1575 mg, about 1325 mg to about 1550 mg, about 1325 mg to about 1525 mg, about 1325 mg to about 1500 mg, about 1325 mg to about 1475 mg, about 1325 mg to about 1450 mg, about 1325 mg to about 1425 mg, about 1325 mg to about 1400 mg, about 1325 mg to about 1375 mg, about 1325 mg to about 1350 mg, about 1350 mg to about 1800 mg, about 1350 mg to about 1775 mg, about 1350 mg to about 1750 mg, about 1350 mg to about 1725 mg, about 1350 mg to about 1700 mg, about 1350 mg to about 1675 mg, about 1350 mg to about 1650 mg, about 1350 mg to about 1625 mg, about 1350 mg to about 1600 mg, about 1350 mg to about 1575 mg, about 1350 mg to about 1550 mg, about 1350 mg to about 1525 mg, about 1350 mg to about 1500 mg, about 1350 mg to about 1475 mg, about 1350 mg to about 1450 mg, about 1350 mg to about 1425 mg, about 1350 mg to about 1400 mg, about 1350 mg to about 1375 mg, about 1375 mg to about 1800 mg, about 1375 mg to about 1775 mg, about 1375 mg to about 1750 mg, about 1375 mg to about 1725 mg, about 1375 mg to about 1700 mg, about 1375 mg to about 1675 mg, about 1375 mg to about 1650 mg, about 1375 mg to about 1625 mg, about 1375 mg to about 1600 mg, about 1375 mg to about 1575 mg, about 1375 mg to about 1550 mg, about 1375 mg to about 1525 mg, about 1375 mg to about 1500 mg, about 1375 mg to about 1475 mg, about 1375 mg to about 1450 mg, about 1375 mg to about 1425 mg, about 1375 mg to about 1400 mg, about 1400 mg to about 1800 mg, about 1400 mg to about 1775 mg, about 1400 mg to about 1750 mg, about 1400 mg to about 1725 mg, about 1400 mg to about 1700 mg, about 1400 mg to about 1675 mg, about 1400 mg to about 1650 mg, about 1400 mg to about 1625 mg, about 1400 mg to about 1600 mg, about 1400 mg to about 1575 mg, about 1400 mg to about 1550 mg, about 1400 mg to about 1525 mg, about 1400 mg to about 1500 mg, about 1400 mg to about 1475 mg, about 1400 mg to about 1450 mg, about 1400 mg to about 1425 mg, about 1425 mg to about 1800 mg, about 1425 mg to about 1775 mg, about 1425 mg to about 1750 mg, about 1425 mg to about 1725 mg, about 1425 mg to about 1700 mg, about 1425 mg to about 1675 mg, about 1425 mg to about 1650 mg, about 1425 mg to about 1625 mg, about 1425 mg to about 1600 mg, about 1425 mg to about 1575 mg, about 1425 mg to about 1550 mg, about 1425 mg to about 1525 mg, about 1425 mg to about 1500 mg, about 1425 mg to about 1475 mg, about 1425 mg to about 1450 mg, about 1450 mg to about 1800 mg, about 1450 mg to about 1775 mg, about 1450 mg to about 1750 mg, about 1450 mg to about 1725 mg, about 1450 mg to about 1700 mg, about 1450 mg to about 1675 mg, about 1450 mg to about 1650 mg, about 1450 mg to about 1625 mg, about 1450 mg to about 1600 mg, about 1450 mg to about 1575 mg, about 1450 mg to about 1550 mg, about 1450 mg to about 1525 mg, about 1450 mg to about 1500 mg, about 1450 mg to about 1475 mg, about 1475 mg to about 1800 mg, about 1475 mg to about 1775 mg, about 1475 mg to about 1750 mg, about 1475 mg to about 1725 mg, about 1475 mg to about 1700 mg, about 1475 mg to about 1675 mg, about 1475 mg to about 1650 mg, about 1475 mg to about 1625 mg, about 1475 mg to about 1600 mg, about 1475 mg to about 1575 mg, about 1475 mg to about 1550 mg, about 1475 mg to about 1525 mg, about 1475 mg to about 1500 mg, about 1500 mg to about 1800 mg, about 1500 mg to about 1775 mg, about 1500 mg to about 1750 mg, about 1500 mg to about 1725 mg, about 1500 mg to about 1700 mg, about 1500 mg to about 1675 mg, about 1500 mg to about 1650 mg, about 1500 mg to about 1625 mg, about 1500 mg to about 1600 mg, about 1500 mg to about 1575 mg, about 1500 mg to about 1550 mg, about 1500 mg to about 1525 mg, about 1525 mg to about 1800 mg, about 1525 mg to about 1775 mg, about 1525 mg to about 1750 mg, about 1525 mg to about 1725 mg, about 1525 mg to about 1700 mg, about 1525 mg to about 1675 mg, about 1525 mg to about 1650 mg, about 1525 mg to about 1625 mg, about 1525 mg to about 1600 mg, about 1525 mg to about 1575 mg, about 1525 mg to about 1550 mg, about 1550 mg to about 1800 mg, about 1550 mg to about 1775 mg, about 1550 mg to about 1750 mg, about 1550 mg to about 1725 mg, about 1550 mg to about 1700 mg, about 1550 mg to about 1675 mg, about 1550 mg to about 1650 mg, about 1550 mg to about 1625 mg, about 1550 mg to about 1600 mg, about 1550 mg to about 1575 mg, about 1575 mg to about 1800 mg, about 1575 mg to about 1775 mg, about 1575 mg to about 1750 mg, about 1575 mg to about 1725 mg, about 1575 mg to about 1700 mg, about 1575 mg to about 1675 mg, about 1575 mg to about 1650 mg, about 1575 mg to about 1625 mg, about 1575 mg to about 1600 mg, about 1600 mg to about 1800 mg, about 1600 mg to about 1775 mg, about 1600 mg to about 1750 mg, about 1600 mg to about 1725 mg, about 1600 mg to about 1700 mg, about 1600 mg to about 1675 mg, about 1600 mg to about 1650 mg, about 1600 mg to about 1625 mg, about 1625 mg to about 1800 mg, about 1625 mg to about 1775 mg, about 1625 mg to about 1750 mg, about 1625 mg to about 1725 mg, about 1625 mg to about 1700 mg, about 1625 mg to about 1675 mg, about 1625 mg to about 1650 mg, about 1650 mg to about 1800 mg, about 1650 mg to about 1775 mg, about 1650 mg to about 1750 mg, about 1650 mg to about 1725 mg, about 1650 mg to about 1700 mg, about 1650 mg to about 1675 mg, about 1675 mg to about 1800 mg, about 1675 mg to about 1775 mg, about 1675 mg to about 1750 mg, about 1675 mg to about 1725 mg, about 1675 mg to about 1700 mg, about 1700 mg to about 1800 mg, about 1700 mg to about 1775 mg, about 1700 mg to about 1750 mg, about 1700 mg to about 1725 mg, about 1725 mg to about 1800 mg, about 1725 mg to about 1775 mg, about 1725 mg to about 1750 mg, about 1750 mg to about 1800 mg, about 1750 mg to about 1775 mg, or about 1775 mg to about 1800 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1) of the anti-tryptase antibody of from about 1350 mg to about 3600 mg (e.g., about 1800 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 1350 mg to about 3600 mg (e.g., about 1800 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 1350 mg to about 3600 mg (e.g., about 1800 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 1350 mg to about 3600 mg, about 1350 mg to about 3550 mg, about 1350 mg to about 3500 mg, about 1350 mg to about 3450 mg, about 1350 mg to about 3400 mg, about 1350 mg to about 3350 mg, about 1350 mg to about 3300 mg, about 1350 mg to about 3250 mg, about 1350 mg to about 3200 mg, about 1350 mg to about 3150 mg, about 1350 mg to about 3100 mg, about 1350 mg to about 3050 mg, about 1350 mg to about 3000 mg, about 1350 mg to about 2950 mg, about 1350 mg to about 2900 mg, about 1350 mg to about 2850 mg, about 1350 mg to about 2800 mg, about 1350 mg to about 2750 mg, about 1350 mg to about 2700 mg, about 1350 mg to about 2650 mg, about 1350 mg to about 2600 mg, about 1350 mg to about 2550 mg, about 1350 mg to about 2500 mg, about 1350 mg to about 2450 mg, about 1350 mg to about 2400 mg, about 1350 mg to about 2350 mg, about 1350 mg to about 2300 mg, about 1350 mg to about 2250 mg, about 1350 mg to about 2200 mg, about 1350 mg to about 2150 mg, about 1350 mg to about 2100 mg, about 1350 mg to about 2050 mg, about 1350 mg to about 2000 mg, about 1350 mg to about 1950 mg, about 1350 mg to about 1900 mg, about 1350 mg to about 1850 mg, about 1350 mg to about 1800 mg, about 1350 mg to about 1750 mg, about 1350 mg to about 1700 mg, about 1350 mg to about 1650 mg, about 1350 mg to about 1600 mg, about 1350 mg to about 1550 mg, about 1350 mg to about 1500 mg, about 1350 mg to about 1450 mg, about 1350 mg to about 1400 mg, about 1400 mg to about 3600 mg, about 1400 mg to about 3550 mg, about 1400 mg to about 3500 mg, about 1400 mg to about 3450 mg, about 1400 mg to about 3400 mg, about 1400 mg to about 3350 mg, about 1400 mg to about 3300 mg, about 1400 mg to about 3250 mg, about 1400 mg to about 3200 mg, about 1400 mg to about 3150 mg, about 1400 mg to about 3100 mg, about 1400 mg to about 3050 mg, about 1400 mg to about 3000 mg, about 1400 mg to about 2950 mg, about 1400 mg to about 2900 mg, about 1400 mg to about 2850 mg, about 1400 mg to about 2800 mg, about 1400 mg to about 2750 mg, about 1400 mg to about 2700 mg, about 1400 mg to about 2650 mg, about 1400 mg to about 2600 mg, about 1400 mg to about 2550 mg, about 1400 mg to about 2500 mg, about 1400 mg to about 2450 mg, about 1400 mg to about 2400 mg, about 1400 mg to about 2350 mg, about 1400 mg to about 2300 mg, about 1400 mg to about 2250 mg, about 1400 mg to about 2200 mg, about 1400 mg to about 2150 mg, about 1400 mg to about 2100 mg, about 1400 mg to about 2050 mg, about 1400 mg to about 2000 mg, about 1400 mg to about 1950 mg, about 1400 mg to about 1900 mg, about 1400 mg to about 1850 mg, about 1400 mg to about 1800 mg, about 1400 mg to about 1750 mg, about 1400 mg to about 1700 mg, about 1400 mg to about 1650 mg, about 1400 mg to about 1600 mg, about 1400 mg to about 1550 mg, about 1400 mg to about 1500 mg, about 1400 mg to about 1450 mg, about 1450 mg to about 3600 mg, about 1450 mg to about 3550 mg, about 1450 mg to about 3500 mg, about 1450 mg to about 3450 mg, about 1450 mg to about 3400 mg, about 1450 mg to about 3350 mg, about 1450 mg to about 3300 mg, about 1450 mg to about 3250 mg, about 1450 mg to about 3200 mg, about 1450 mg to about 3150 mg, about 1450 mg to about 3100 mg, about 1450 mg to about 3050 mg, about 1450 mg to about 3000 mg, about 1450 mg to about 2950 mg, about 1450 mg to about 2900 mg, about 1450 mg to about 2850 mg, about 1450 mg to about 2800 mg, about 1450 mg to about 2750 mg, about 1450 mg to about 2700 mg, about 1450 mg to about 2650 mg, about 1450 mg to about 2600 mg, about 1450 mg to about 2550 mg, about 1450 mg to about 2500 mg, about 1450 mg to about 2450 mg, about 1450 mg to about 2400 mg, about 1450 mg to about 2350 mg, about 1450 mg to about 2300 mg, about 1450 mg to about 2250 mg, about 1450 mg to about 2200 mg, about 1450 mg to about 2150 mg, about 1450 mg to about 2100 mg, about 1450 mg to about 2050 mg, about 1450 mg to about 2000 mg, about 1450 mg to about 1950 mg, about 1450 mg to about 1900 mg, about 1450 mg to about 1850 mg, about 1450 mg to about 1800 mg, about 1450 mg to about 1750 mg, about 1450 mg to about 1700 mg, about 1450 mg to about 1650 mg, about 1450 mg to about 1600 mg, about 1450 mg to about 1550 mg, about 1450 mg to about 1500 mg, about 1500 mg to about 3600 mg, about 1500 mg to about 3550 mg, about 1500 mg to about 3500 mg, about 1500 mg to about 3450 mg, about 1500 mg to about 3400 mg, about 1500 mg to about 3350 mg, about 1500 mg to about 3300 mg, about 1500 mg to about 3250 mg, about 1500 mg to about 3200 mg, about 1500 mg to about 3150 mg, about 1500 mg to about 3100 mg, about 1500 mg to about 3050 mg, about 1500 mg to about 3000 mg, about 1500 mg to about 2950 mg, about 1500 mg to about 2900 mg, about 1500 mg to about 2850 mg, about 1500 mg to about 2800 mg, about 1500 mg to about 2750 mg, about 1500 mg to about 2700 mg, about 1500 mg to about 2650 mg, about 1500 mg to about 2600 mg, about 1500 mg to about 2550 mg, about 1500 mg to about 2500 mg, about 1500 mg to about 2450 mg, about 1500 mg to about 2400 mg, about 1500 mg to about 2350 mg, about 1500 mg to about 2300 mg, about 1500 mg to about 2250 mg, about 1500 mg to about 2200 mg, about 1500 mg to about 2150 mg, about 1500 mg to about 2100 mg, about 1500 mg to about 2050 mg, about 1500 mg to about 2000 mg, about 1500 mg to about 1950 mg, about 1500 mg to about 1900 mg, about 1500 mg to about 1850 mg, about 1500 mg to about 1800 mg, about 1500 mg to about 1750 mg, about 1500 mg to about 1700 mg, about 1500 mg to about 1650 mg, about 1500 mg to about 1600 mg, about 1500 mg to about 1550 mg, about 1550 mg to about 3600 mg, about 1550 mg to about 3550 mg, about 1550 mg to about 3500 mg, about 1550 mg to about 3450 mg, about 1550 mg to about 3400 mg, about 1550 mg to about 3350 mg, about 1550 mg to about 3300 mg, about 1550 mg to about 3250 mg, about 1550 mg to about 3200 mg, about 1550 mg to about 3150 mg, about 1550 mg to about 3100 mg, about 1550 mg to about 3050 mg, about 1550 mg to about 3000 mg, about 1550 mg to about 2950 mg, about 1550 mg to about 2900 mg, about 1550 mg to about 2850 mg, about 1550 mg to about 2800 mg, about 1550 mg to about 2750 mg, about 1550 mg to about 2700 mg, about 1550 mg to about 2650 mg, about 1550 mg to about 2600 mg, about 1550 mg to about 2550 mg, about 1550 mg to about 2500 mg, about 1550 mg to about 2450 mg, about 1550 mg to about 2400 mg, about 1550 mg to about 2350 mg, about 1550 mg to about 2300 mg, about 1550 mg to about 2250 mg, about 1550 mg to about 2200 mg, about 1550 mg to about 2150 mg, about 1550 mg to about 2100 mg, about 1550 mg to about 2050 mg, about 1550 mg to about 2000 mg, about 1550 mg to about 1950 mg, about 1550 mg to about 1900 mg, about 1550 mg to about 1850 mg, about 1550 mg to about 1800 mg, about 1550 mg to about 1750 mg, about 1550 mg to about 1700 mg, about 1550 mg to about 1650 mg, about 1550 mg to about 1600 mg, about 1600 mg to about 3600 mg, about 1600 mg to about 3550 mg, about 1600 mg to about 3500 mg, about 1600 mg to about 3450 mg, about 1600 mg to about 3400 mg, about 1600 mg to about 3350 mg, about 1600 mg to about 3300 mg, about 1600 mg to about 3250 mg, about 1600 mg to about 3200 mg, about 1600 mg to about 3150 mg, about 1600 mg to about 3100 mg, about 1600 mg to about 3050 mg, about 1600 mg to about 3000 mg, about 1600 mg to about 2950 mg, about 1600 mg to about 2900 mg, about 1600 mg to about 2850 mg, about 1600 mg to about 2800 mg, about 1600 mg to about 2750 mg, about 1600 mg to about 2700 mg, about 1600 mg to about 2650 mg, about 1600 mg to about 2600 mg, about 1600 mg to about 2550 mg, about 1600 mg to about 2500 mg, about 1600 mg to about 2450 mg, about 1600 mg to about 2400 mg, about 1600 mg to about 2350 mg, about 1600 mg to about 2300 mg, about 1600 mg to about 2250 mg, about 1600 mg to about 2200 mg, about 1600 mg to about 2150 mg, about 1600 mg to about 2100 mg, about 1600 mg to about 2050 mg, about 1600 mg to about 2000 mg, about 1600 mg to about 1950 mg, about 1600 mg to about 1900 mg, about 1600 mg to about 1850 mg, about 1600 mg to about 1800 mg, about 1600 mg to about 1750 mg, about 1600 mg to about 1700 mg, about 1600 mg to about 1650 mg, about 1650 mg to about 3600 mg, about 1650 mg to about 3550 mg, about 1650 mg to about 3500 mg, about 1650 mg to about 3450 mg, about 1650 mg to about 3400 mg, about 1650 mg to about 3350 mg, about 1650 mg to about 3300 mg, about 1650 mg to about 3250 mg, about 1650 mg to about 3200 mg, about 1650 mg to about 3150 mg, about 1650 mg to about 3100 mg, about 1650 mg to about 3050 mg, about 1650 mg to about 3000 mg, about 1650 mg to about 2950 mg, about 1650 mg to about 2900 mg, about 1650 mg to about 2850 mg, about 1650 mg to about 2800 mg, about 1650 mg to about 2750 mg, about 1650 mg to about 2700 mg, about 1650 mg to about 2650 mg, about 1650 mg to about 2600 mg, about 1650 mg to about 2550 mg, about 1650 mg to about 2500 mg, about 1650 mg to about 2450 mg, about 1650 mg to about 2400 mg, about 1650 mg to about 2350 mg, about 1650 mg to about 2300 mg, about 1650 mg to about 2250 mg, about 1650 mg to about 2200 mg, about 1650 mg to about 2150 mg, about 1650 mg to about 2100 mg, about 1650 mg to about 2050 mg, about 1650 mg to about 2000 mg, about 1650 mg to about 1950 mg, about 1650 mg to about 1900 mg, about 1650 mg to about 1850 mg, about 1650 mg to about 1800 mg, about 1650 mg to about 1750 mg, about 1650 mg to about 1700 mg, about 1700 mg to about 3600 mg, about 1700 mg to about 3550 mg, about 1700 mg to about 3500 mg, about 1700 mg to about 3450 mg, about 1700 mg to about 3400 mg, about 1700 mg to about 3350 mg, about 1700 mg to about 3300 mg, about 1700 mg to about 3250 mg, about 1700 mg to about 3200 mg, about 1700 mg to about 3150 mg, about 1700 mg to about 3100 mg, about 1700 mg to about 3050 mg, about 1700 mg to about 3000 mg, about 1700 mg to about 2950 mg, about 1700 mg to about 2900 mg, about 1700 mg to about 2850 mg, about 1700 mg to about 2800 mg, about 1700 mg to about 2750 mg, about 1700 mg to about 2700 mg, about 1700 mg to about 2650 mg, about 1700 mg to about 2600 mg, about 1700 mg to about 2550 mg, about 1700 mg to about 2500 mg, about 1700 mg to about 2450 mg, about 1700 mg to about 2400 mg, about 1700 mg to about 2350 mg, about 1700 mg to about 2300 mg, about 1700 mg to about 2250 mg, about 1700 mg to about 2200 mg, about 1700 mg to about 2150 mg, about 1700 mg to about 2100 mg, about 1700 mg to about 2050 mg, about 1700 mg to about 2000 mg, about 1700 mg to about 1950 mg, about 1700 mg to about 1900 mg, about 1700 mg to about 1850 mg, about 1700 mg to about 1800 mg, about 1700 mg to about 1750 mg, about 1750 mg to about 3600 mg, about 1750 mg to about 3550 mg, about 1750 mg to about 3500 mg, about 1750 mg to about 3450 mg, about 1750 mg to about 3400 mg, about 1750 mg to about 3350 mg, about 1750 mg to about 3300 mg, about 1750 mg to about 3250 mg, about 1750 mg to about 3200 mg, about 1750 mg to about 3150 mg, about 1750 mg to about 3100 mg, about 1750 mg to about 3050 mg, about 1750 mg to about 3000 mg, about 1750 mg to about 2950 mg, about 1750 mg to about 2900 mg, about 1750 mg to about 2850 mg, about 1750 mg to about 2800 mg, about 1750 mg to about 2750 mg, about 1750 mg to about 2700 mg, about 1750 mg to about 2650 mg, about 1750 mg to about 2600 mg, about 1750 mg to about 2550 mg, about 1750 mg to about 2500 mg, about 1750 mg to about 2450 mg, about 1750 mg to about 2400 mg, about 1750 mg to about 2350 mg, about 1750 mg to about 2300 mg, about 1750 mg to about 2250 mg, about 1750 mg to about 2200 mg, about 1750 mg to about 2150 mg, about 1750 mg to about 2100 mg, about 1750 mg to about 2050 mg, about 1750 mg to about 2000 mg, about 1750 mg to about 1950 mg, about 1750 mg to about 1900 mg, about 1750 mg to about 1850 mg, about 1750 mg to about 1800 mg, about 1800 mg to about 3600 mg, about 1800 mg to about 3550 mg, about 1800 mg to about 3500 mg, about 1800 mg to about 3450 mg, about 1800 mg to about 3400 mg, about 1800 mg to about 3350 mg, about 1800 mg to about 3300 mg, about 1800 mg to about 3250 mg, about 1800 mg to about 3200 mg, about 1800 mg to about 3150 mg, about 1800 mg to about 3100 mg, about 1800 mg to about 3050 mg, about 1800 mg to about 3000 mg, about 1800 mg to about 2950 mg, about 1800 mg to about 2900 mg, about 1800 mg to about 2850 mg, about 1800 mg to about 2800 mg, about 1800 mg to about 2750 mg, about 1800 mg to about 2700 mg, about 1800 mg to about 2650 mg, about 1800 mg to about 2600 mg, about 1800 mg to about 2550 mg, about 1800 mg to about 2500 mg, about 1800 mg to about 2450 mg, about 1800 mg to about 2400 mg, about 1800 mg to about 2350 mg, about 1800 mg to about 2300 mg, about 1800 mg to about 2250 mg, about 1800 mg to about 2200 mg, about 1800 mg to about 2150 mg, about 1800 mg to about 2100 mg, about 1800 mg to about 2050 mg, about 1800 mg to about 2000 mg, about 1800 mg to about 1950 mg, about 1800 mg to about 1900 mg, about 1800 mg to about 1850 mg, about 1850 mg to about 3600 mg, about 1850 mg to about 3550 mg, about 1850 mg to about 3500 mg, about 1850 mg to about 3450 mg, about 1850 mg to about 3400 mg, about 1850 mg to about 3350 mg, about 1850 mg to about 3300 mg, about 1850 mg to about 3250 mg, about 1850 mg to about 3200 mg, about 1850 mg to about 3150 mg, about 1850 mg to about 3100 mg, about 1850 mg to about 3050 mg, about 1850 mg to about 3000 mg, about 1850 mg to about 2950 mg, about 1850 mg to about 2900 mg, about 1850 mg to about 2850 mg, about 1850 mg to about 2800 mg, about 1850 mg to about 2750 mg, about 1850 mg to about 2700 mg, about 1850 mg to about 2650 mg, about 1850 mg to about 2600 mg, about 1850 mg to about 2550 mg, about 1850 mg to about 2500 mg, about 1850 mg to about 2450 mg, about 1850 mg to about 2400 mg, about 1850 mg to about 2350 mg, about 1850 mg to about 2300 mg, about 1850 mg to about 2250 mg, about 1850 mg to about 2200 mg, about 1850 mg to about 2150 mg, about 1850 mg to about 2100 mg, about 1850 mg to about 2050 mg, about 1850 mg to about 2000 mg, about 1850 mg to about 1950 mg, about 1850 mg to about 1900 mg, about 1900 mg to about 3600 mg, about 1900 mg to about 3550 mg, about 1900 mg to about 3500 mg, about 1900 mg to about 3450 mg, about 1900 mg to about 3400 mg, about 1900 mg to about 3350 mg, about 1900 mg to about 3300 mg, about 1900 mg to about 3250 mg, about 1900 mg to about 3200 mg, about 1900 mg to about 3150 mg, about 1900 mg to about 3100 mg, about 1900 mg to about 3050 mg, about 1900 mg to about 3000 mg, about 1900 mg to about 2950 mg, about 1900 mg to about 2900 mg, about 1900 mg to about 2850 mg, about 1900 mg to about 2800 mg, about 1900 mg to about 2750 mg, about 1900 mg to about 2700 mg, about 1900 mg to about 2650 mg, about 1900 mg to about 2600 mg, about 1900 mg to about 2550 mg, about 1900 mg to about 2500 mg, about 1900 mg to about 2450 mg, about 1900 mg to about 2400 mg, about 1900 mg to about 2350 mg, about 1900 mg to about 2300 mg, about 1900 mg to about 2250 mg, about 1900 mg to about 2200 mg, about 1900 mg to about 2150 mg, about 1900 mg to about 2100 mg, about 1900 mg to about 2050 mg, about 1900 mg to about 2000 mg, about 1900 mg to about 1950 mg, about 1950 mg to about 3600 mg, about 1950 mg to about 3550 mg, about 1950 mg to about 3500 mg, about 1950 mg to about 3450 mg, about 1950 mg to about 3400 mg, about 1950 mg to about 3350 mg, about 1950 mg to about 3300 mg, about 1950 mg to about 3250 mg, about 1950 mg to about 3200 mg, about 1950 mg to about 3150 mg, about 1950 mg to about 3100 mg, about 1950 mg to about 3050 mg, about 1950 mg to about 3000 mg, about 1950 mg to about 2950 mg, about 1950 mg to about 2900 mg, about 1950 mg to about 2850 mg, about 1950 mg to about 2800 mg, about 1950 mg to about 2750 mg, about 1950 mg to about 2700 mg, about 1950 mg to about 2650 mg, about 1950 mg to about 2600 mg, about 1950 mg to about 2550 mg, about 1950 mg to about 2500 mg, about 1950 mg to about 2450 mg, about 1950 mg to about 2400 mg, about 1950 mg to about 2350 mg, about 1950 mg to about 2300 mg, about 1950 mg to about 2250 mg, about 1950 mg to about 2200 mg, about 1950 mg to about 2150 mg, about 1950 mg to about 2100 mg, about 1950 mg to about 2050 mg, about 1950 mg to about 2000 mg, about 2000 mg to about 3600 mg, about 2000 mg to about 3550 mg, about 2000 mg to about 3500 mg, about 2000 mg to about 3450 mg, about 2000 mg to about 3400 mg, about 2000 mg to about 3350 mg, about 2000 mg to about 3300 mg, about 2000 mg to about 3250 mg, about 2000 mg to about 3200 mg, about 2000 mg to about 3150 mg, about 2000 mg to about 3100 mg, about 2000 mg to about 3050 mg, about 2000 mg to about 3000 mg, about 2000 mg to about 2950 mg, about 2000 mg to about 2900 mg, about 2000 mg to about 2850 mg, about 2000 mg to about 2800 mg, about 2000 mg to about 2750 mg, about 2000 mg to about 2700 mg, about 2000 mg to about 2650 mg, about 2000 mg to about 2600 mg, about 2000 mg to about 2550 mg, about 2000 mg to about 2500 mg, about 2000 mg to about 2450 mg, about 2000 mg to about 2400 mg, about 2000 mg to about 2350 mg, about 2000 mg to about 2300 mg, about 2000 mg to about 2250 mg, about 2000 mg to about 2200 mg, about 2000 mg to about 2150 mg, about 2000 mg to about 2100 mg, about 2000 mg to about 2050 mg, about 2050 mg to about 3600 mg, about 2050 mg to about 3550 mg, about 2050 mg to about 3500 mg, about 2050 mg to about 3450 mg, about 2050 mg to about 3400 mg, about 2050 mg to about 3350 mg, about 2050 mg to about 3300 mg, about 2050 mg to about 3250 mg, about 2050 mg to about 3200 mg, about 2050 mg to about 3150 mg, about 2050 mg to about 3100 mg, about 2050 mg to about 3050 mg, about 2050 mg to about 3000 mg, about 2050 mg to about 2950 mg, about 2050 mg to about 2900 mg, about 2050 mg to about 2850 mg, about 2050 mg to about 2800 mg, about 2050 mg to about 2750 mg, about 2050 mg to about 2700 mg, about 2050 mg to about 2650 mg, about 2050 mg to about 2600 mg, about 2050 mg to about 2550 mg, about 2050 mg to about 2500 mg, about 2050 mg to about 2450 mg, about 2050 mg to about 2400 mg, about 2050 mg to about 2350 mg, about 2050 mg to about 2300 mg, about 2050 mg to about 2250 mg, about 2050 mg to about 2200 mg, about 2050 mg to about 2150 mg, about 2050 mg to about 2100 mg, about 2100 mg to about 3600 mg, about 2100 mg to about 3550 mg, about 2100 mg to about 3500 mg, about 2100 mg to about 3450 mg, about 2100 mg to about 3400 mg, about 2100 mg to about 3350 mg, about 2100 mg to about 3300 mg, about 2100 mg to about 3250 mg, about 2100 mg to about 3200 mg, about 2100 mg to about 3150 mg, about 2100 mg to about 3100 mg, about 2100 mg to about 3050 mg, about 2100 mg to about 3000 mg, about 2100 mg to about 2950 mg, about 2100 mg to about 2900 mg, about 2100 mg to about 2850 mg, about 2100 mg to about 2800 mg, about 2100 mg to about 2750 mg, about 2100 mg to about 2700 mg, about 2100 mg to about 2650 mg, about 2100 mg to about 2600 mg, about 2100 mg to about 2550 mg, about 2100 mg to about 2500 mg, about 2100 mg to about 2450 mg, about 2100 mg to about 2400 mg, about 2100 mg to about 2350 mg, about 2100 mg to about 2300 mg, about 2100 mg to about 2250 mg, about 2100 mg to about 2200 mg, about 2100 mg to about 2150 mg, about 2150 mg to about 3600 mg, about 2150 mg to about 3550 mg, about 2150 mg to about 3500 mg, about 2150 mg to about 3450 mg, about 2150 mg to about 3400 mg, about 2150 mg to about 3350 mg, about 2150 mg to about 3300 mg, about 2150 mg to about 3250 mg, about 2150 mg to about 3200 mg, about 2150 mg to about 3150 mg, about 2150 mg to about 3100 mg, about 2150 mg to about 3050 mg, about 2150 mg to about 3000 mg, about 2150 mg to about 2950 mg, about 2150 mg to about 2900 mg, about 2150 mg to about 2850 mg, about 2150 mg to about 2800 mg, about 2150 mg to about 2750 mg, about 2150 mg to about 2700 mg, about 2150 mg to about 2650 mg, about 2150 mg to about 2600 mg, about 2150 mg to about 2550 mg, about 2150 mg to about 2500 mg, about 2150 mg to about 2450 mg, about 2150 mg to about 2400 mg, about 2150 mg to about 2350 mg, about 2150 mg to about 2300 mg, about 2150 mg to about 2250 mg, about 2150 mg to about 2200 mg, about 2200 mg to about 3600 mg, about 2200 mg to about 3550 mg, about 2200 mg to about 3500 mg, about 2200 mg to about 3450 mg, about 2200 mg to about 3400 mg, about 2200 mg to about 3350 mg, about 2200 mg to about 3300 mg, about 2200 mg to about 3250 mg, about 2200 mg to about 3200 mg, about 2200 mg to about 3150 mg, about 2200 mg to about 3100 mg, about 2200 mg to about 3050 mg, about 2200 mg to about 3000 mg, about 2200 mg to about 2950 mg, about 2200 mg to about 2900 mg, about 2200 mg to about 2850 mg, about 2200 mg to about 2800 mg, about 2200 mg to about 2750 mg, about 2200 mg to about 2700 mg, about 2200 mg to about 2650 mg, about 2200 mg to about 2600 mg, about 2200 mg to about 2550 mg, about 2200 mg to about 2500 mg, about 2200 mg to about 2450 mg, about 2200 mg to about 2400 mg, about 2200 mg to about 2350 mg, about 2200 mg to about 2300 mg, about 2200 mg to about 2250 mg, about 2250 mg to about 3600 mg, about 2250 mg to about 3550 mg, about 2250 mg to about 3500 mg, about 2250 mg to about 3450 mg, about 2250 mg to about 3400 mg, about 2250 mg to about 3350 mg, about 2250 mg to about 3300 mg, about 2250 mg to about 3250 mg, about 2250 mg to about 3200 mg, about 2250 mg to about 3150 mg, about 2250 mg to about 3100 mg, about 2250 mg to about 3050 mg, about 2250 mg to about 3000 mg, about 2250 mg to about 2950 mg, about 2250 mg to about 2900 mg, about 2250 mg to about 2850 mg, about 2250 mg to about 2800 mg, about 2250 mg to about 2750 mg, about 2250 mg to about 2700 mg, about 2250 mg to about 2650 mg, about 2250 mg to about 2600 mg, about 2250 mg to about 2550 mg, about 2250 mg to about 2500 mg, about 2250 mg to about 2450 mg, about 2250 mg to about 2400 mg, about 2250 mg to about 2350 mg, about 2250 mg to about 2300 mg, about 2300 mg to about 3600 mg, about 2300 mg to about 3550 mg, about 2300 mg to about 3500 mg, about 2300 mg to about 3450 mg, about 2300 mg to about 3400 mg, about 2300 mg to about 3350 mg, about 2300 mg to about 3300 mg, about 2300 mg to about 3250 mg, about 2300 mg to about 3200 mg, about 2300 mg to about 3150 mg, about 2300 mg to about 3100 mg, about 2300 mg to about 3050 mg, about 2300 mg to about 3000 mg, about 2300 mg to about 2950 mg, about 2300 mg to about 2900 mg, about 2300 mg to about 2850 mg, about 2300 mg to about 2800 mg, about 2300 mg to about 2750 mg, about 2300 mg to about 2700 mg, about 2300 mg to about 2650 mg, about 2300 mg to about 2600 mg, about 2300 mg to about 2550 mg, about 2300 mg to about 2500 mg, about 2300 mg to about 2450 mg, about 2300 mg to about 2400 mg, about 2300 mg to about 2350 mg, about 2350 mg to about 3600 mg, about 2350 mg to about 3550 mg, about 2350 mg to about 3500 mg, about 2350 mg to about 3450 mg, about 2350 mg to about 3400 mg, about 2350 mg to about 3350 mg, about 2350 mg to about 3300 mg, about 2350 mg to about 3250 mg, about 2350 mg to about 3200 mg, about 2350 mg to about 3150 mg, about 2350 mg to about 3100 mg, about 2350 mg to about 3050 mg, about 2350 mg to about 3000 mg, about 2350 mg to about 2950 mg, about 2350 mg to about 2900 mg, about 2350 mg to about 2850 mg, about 2350 mg to about 2800 mg, about 2350 mg to about 2750 mg, about 2350 mg to about 2700 mg, about 2350 mg to about 2650 mg, about 2350 mg to about 2600 mg, about 2350 mg to about 2550 mg, about 2350 mg to about 2500 mg, about 2350 mg to about 2450 mg, about 2350 mg to about 2400 mg, about 2400 mg to about 3600 mg, about 2400 mg to about 3550 mg, about 2400 mg to about 3500 mg, about 2400 mg to about 3450 mg, about 2400 mg to about 3400 mg, about 2400 mg to about 3350 mg, about 2400 mg to about 3300 mg, about 2400 mg to about 3250 mg, about 2400 mg to about 3200 mg, about 2400 mg to about 3150 mg, about 2400 mg to about 3100 mg, about 2400 mg to about 3050 mg, about 2400 mg to about 3000 mg, about 2400 mg to about 2950 mg, about 2400 mg to about 2900 mg, about 2400 mg to about 2850 mg, about 2400 mg to about 2800 mg, about 2400 mg to about 2750 mg, about 2400 mg to about 2700 mg, about 2400 mg to about 2650 mg, about 2400 mg to about 2600 mg, about 2400 mg to about 2550 mg, about 2400 mg to about 2500 mg, about 2400 mg to about 2450 mg, about 2450 mg to about 3600 mg, about 2450 mg to about 3550 mg, about 2450 mg to about 3500 mg, about 2450 mg to about 3450 mg, about 2450 mg to about 3400 mg, about 2450 mg to about 3350 mg, about 2450 mg to about 3300 mg, about 2450 mg to about 3250 mg, about 2450 mg to about 3200 mg, about 2450 mg to about 3150 mg, about 2450 mg to about 3100 mg, about 2450 mg to about 3050 mg, about 2450 mg to about 3000 mg, about 2450 mg to about 2950 mg, about 2450 mg to about 2900 mg, about 2450 mg to about 2850 mg, about 2450 mg to about 2800 mg, about 2450 mg to about 2750 mg, about 2450 mg to about 2700 mg, about 2450 mg to about 2650 mg, about 2450 mg to about 2600 mg, about 2450 mg to about 2550 mg, about 2450 mg to about 2500 mg, about 2500 mg to about 3600 mg, about 2500 mg to about 3550 mg, about 2500 mg to about 3500 mg, about 2500 mg to about 3450 mg, about 2500 mg to about 3400 mg, about 2500 mg to about 3350 mg, about 2500 mg to about 3300 mg, about 2500 mg to about 3250 mg, about 2500 mg to about 3200 mg, about 2500 mg to about 3150 mg, about 2500 mg to about 3100 mg, about 2500 mg to about 3050 mg, about 2500 mg to about 3000 mg, about 2500 mg to about 2950 mg, about 2500 mg to about 2900 mg, about 2500 mg to about 2850 mg, about 2500 mg to about 2800 mg, about 2500 mg to about 2750 mg, about 2500 mg to about 2700 mg, about 2500 mg to about 2650 mg, about 2500 mg to about 2600 mg, about 2500 mg to about 2550 mg, about 2550 mg to about 3600 mg, about 2550 mg to about 3550 mg, about 2550 mg to about 3500 mg, about 2550 mg to about 3450 mg, about 2550 mg to about 3400 mg, about 2550 mg to about 3350 mg, about 2550 mg to about 3300 mg, about 2550 mg to about 3250 mg, about 2550 mg to about 3200 mg, about 2550 mg to about 3150 mg, about 2550 mg to about 3100 mg, about 2550 mg to about 3050 mg, about 2550 mg to about 3000 mg, about 2550 mg to about 2950 mg, about 2550 mg to about 2900 mg, about 2550 mg to about 2850 mg, about 2550 mg to about 2800 mg, about 2550 mg to about 2750 mg, about 2550 mg to about 2700 mg, about 2550 mg to about 2650 mg, about 2550 mg to about 2600 mg, about 2600 mg to about 3600 mg, about 2600 mg to about 3550 mg, about 2600 mg to about 3500 mg, about 2600 mg to about 3450 mg, about 2600 mg to about 3400 mg, about 2600 mg to about 3350 mg, about 2600 mg to about 3300 mg, about 2600 mg to about 3250 mg, about 2600 mg to about 3200 mg, about 2600 mg to about 3150 mg, about 2600 mg to about 3100 mg, about 2600 mg to about 3050 mg, about 2600 mg to about 3000 mg, about 2600 mg to about 2950 mg, about 2600 mg to about 2900 mg, about 2600 mg to about 2850 mg, about 2600 mg to about 2800 mg, about 2600 mg to about 2750 mg, about 2600 mg to about 2700 mg, about 2600 mg to about 2650 mg, about 2650 mg to about 3600 mg, about 2650 mg to about 3550 mg, about 2650 mg to about 3500 mg, about 2650 mg to about 3450 mg, about 2650 mg to about 3400 mg, about 2650 mg to about 3350 mg, about 2650 mg to about 3300 mg, about 2650 mg to about 3250 mg, about 2650 mg to about 3200 mg, about 2650 mg to about 3150 mg, about 2650 mg to about 3100 mg, about 2650 mg to about 3050 mg, about 2650 mg to about 3000 mg, about 2650 mg to about 2950 mg, about 2650 mg to about 2900 mg, about 2650 mg to about 2850 mg, about 2650 mg to about 2800 mg, about 2650 mg to about 2750 mg, about 2650 mg to about 2700 mg, about 2700 mg to about 3600 mg, about 2700 mg to about 3550 mg, about 2700 mg to about 3500 mg, about 2700 mg to about 3450 mg, about 2700 mg to about 3400 mg, about 2700 mg to about 3350 mg, about 2700 mg to about 3300 mg, about 2700 mg to about 3250 mg, about 2700 mg to about 3200 mg, about 2700 mg to about 3150 mg, about 2700 mg to about 3100 mg, about 2700 mg to about 3050 mg, about 2700 mg to about 3000 mg, about 2700 mg to about 2950 mg, about 2700 mg to about 2900 mg, about 2700 mg to about 2850 mg, about 2700 mg to about 2800 mg, about 2700 mg to about 2750 mg, about 2750 mg to about 3600 mg, about 2750 mg to about 3550 mg, about 2750 mg to about 3500 mg, about 2750 mg to about 3450 mg, about 2750 mg to about 3400 mg, about 2750 mg to about 3350 mg, about 2750 mg to about 3300 mg, about 2750 mg to about 3250 mg, about 2750 mg to about 3200 mg, about 2750 mg to about 3150 mg, about 2750 mg to about 3100 mg, about 2750 mg to about 3050 mg, about 2750 mg to about 3000 mg, about 2750 mg to about 2950 mg, about 2750 mg to about 2900 mg, about 2750 mg to about 2850 mg, about 2750 mg to about 2800 mg, about 2800 mg to about 3600 mg, about 2800 mg to about 3550 mg, about 2800 mg to about 3500 mg, about 2800 mg to about 3450 mg, about 2800 mg to about 3400 mg, about 2800 mg to about 3350 mg, about 2800 mg to about 3300 mg, about 2800 mg to about 3250 mg, about 2800 mg to about 3200 mg, about 2800 mg to about 3150 mg, about 2800 mg to about 3100 mg, about 2800 mg to about 3050 mg, about 2800 mg to about 3000 mg, about 2800 mg to about 2950 mg, about 2800 mg to about 2900 mg, about 2800 mg to about 2850 mg, about 2850 mg to about 3600 mg, about 2850 mg to about 3550 mg, about 2850 mg to about 3500 mg, about 2850 mg to about 3450 mg, about 2850 mg to about 3400 mg, about 2850 mg to about 3350 mg, about 2850 mg to about 3300 mg, about 2850 mg to about 3250 mg, about 2850 mg to about 3200 mg, about 2850 mg to about 3150 mg, about 2850 mg to about 3100 mg, about 2850 mg to about 3050 mg, about 2850 mg to about 3000 mg, about 2850 mg to about 2950 mg, about 2850 mg to about 2900 mg, about 2900 mg to about 3600 mg, about 2900 mg to about 3550 mg, about 2900 mg to about 3500 mg, about 2900 mg to about 3450 mg, about 2900 mg to about 3400 mg, about 2900 mg to about 3350 mg, about 2900 mg to about 3300 mg, about 2900 mg to about 3250 mg, about 2900 mg to about 3200 mg, about 2900 mg to about 3150 mg, about 2900 mg to about 3100 mg, about 2900 mg to about 3050 mg, about 2900 mg to about 3000 mg, about 2900 mg to about 2950 mg, about 2950 mg to about 3600 mg, about 2950 mg to about 3550 mg, about 2950 mg to about 3500 mg, about 2950 mg to about 3450 mg, about 2950 mg to about 3400 mg, about 2950 mg to about 3350 mg, about 2950 mg to about 3300 mg, about 2950 mg to about 3250 mg, about 2950 mg to about 3200 mg, about 2950 mg to about 3150 mg, about 2950 mg to about 3100 mg, about 2950 mg to about 3050 mg, about 2950 mg to about 3000 mg, about 3000 mg to about 3600 mg, about 3000 mg to about 3550 mg, about 3000 mg to about 3500 mg, about 3000 mg to about 3450 mg, about 3000 mg to about 3400 mg, about 3000 mg to about 3350 mg, about 3000 mg to about 3300 mg, about 3000 mg to about 3250 mg, about 3000 mg to about 3200 mg, about 3000 mg to about 3150 mg, about 3000 mg to about 3100 mg, about 3000 mg to about 3050 mg, about 3050 mg to about 3600 mg, about 3050 mg to about 3550 mg, about 3050 mg to about 3500 mg, about 3050 mg to about 3450 mg, about 3050 mg to about 3400 mg, about 3050 mg to about 3350 mg, about 3050 mg to about 3300 mg, about 3050 mg to about 3250 mg, about 3050 mg to about 3200 mg, about 3050 mg to about 3150 mg, about 3050 mg to about 3100 mg, about 3100 mg to about 3600 mg, about 3100 mg to about 3550 mg, about 3100 mg to about 3500 mg, about 3100 mg to about 3450 mg, about 3100 mg to about 3400 mg, about 3100 mg to about 3350 mg, about 3100 mg to about 3300 mg, about 3100 mg to about 3250 mg, about 3100 mg to about 3200 mg, about 3100 mg to about 3150 mg, about 3150 mg to about 3600 mg, about 3150 mg to about 3550 mg, about 3150 mg to about 3500 mg, about 3150 mg to about 3450 mg, about 3150 mg to about 3400 mg, about 3150 mg to about 3350 mg, about 3150 mg to about 3300 mg, about 3150 mg to about 3250 mg, about 3150 mg to about 3200 mg, about 3200 mg to about 3600 mg, about 3200 mg to about 3550 mg, about 3200 mg to about 3500 mg, about 3200 mg to about 3450 mg, about 3200 mg to about 3400 mg, about 3200 mg to about 3350 mg, about 3200 mg to about 3300 mg, about 3200 mg to about 3250 mg, about 3250 mg to about 3600 mg, about 3250 mg to about 3550 mg, about 3250 mg to about 3500 mg, about 3250 mg to about 3450 mg, about 3250 mg to about 3400 mg, about 3250 mg to about 3350 mg, about 3250 mg to about 3300 mg, about 3300 mg to about 3600 mg, about 3300 mg to about 3550 mg, about 3300 mg to about 3500 mg, about 3300 mg to about 3450 mg, about 3300 mg to about 3400 mg, about 3300 mg to about 3350 mg, about 3350 mg to about 3600 mg, about 3350 mg to about 3550 mg, about 3350 mg to about 3500 mg, about 3350 mg to about 3450 mg, about 3350 mg to about 3400 mg, about 3400 mg to about 3600 mg, about 3400 mg to about 3550 mg, about 3400 mg to about 3500 mg, about 3400 mg to about 3450 mg, about 3450 mg to about 3600 mg, about 3450 mg to about 3550 mg, about 3450 mg to about 3500 mg, about 3500 mg to about 3600 mg, about 3500 mg to about 3550 mg, or about 3550 mg to about 3600 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of from about 1800 mg to about 4000 mg (e.g., about 3600 mg). The C1D1 may be administered, for example, IV or SC. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of from about 1800 mg to about 4000 mg (e.g., about 3600 mg). In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of from about 1800 mg to about 4000 mg (e.g., about 3600 mg). In some aspects, the C1D1 is administered IV. In other aspects, the C1D1 is administered SC (e.g., by a pump (e.g., by a patch pump).

For example, in any of the preceding aspects, the first dose (C1D1 ) of the anti-tryptase antibody, and/or any additional doses of the anti-tryptase antibody, may be about 1800 mg to about 4000 mg, about 1800 mg to about 3900 mg, about 1800 mg to about 3800 mg, about 1800 mg to about 3700 mg, about 1800 mg to about 3600 mg, about 1800 mg to about 3500 mg, about 1800 mg to about 3400 mg, about 1800 mg to about 3300 mg, about 1800 mg to about 3200 mg, about 1800 mg to about 3100 mg, about 1800 mg to about 3000 mg, about 1800 mg to about 2900 mg, about 1800 mg to about 2800 mg, about 1800 mg to about 2700 mg, about 1800 mg to about 2600 mg, about 1800 mg to about 2500 mg, about 1800 mg to about 2400 mg, about 1800 mg to about 2300 mg, about 1800 mg to about 2200 mg, about 1800 mg to about 2100 mg, about 1800 mg to about 2000 mg, about 1800 mg to about 1900 mg, about 1900 mg to about 4000 mg, about 1900 mg to about 3900 mg, about 1900 mg to about 3800 mg, about 1900 mg to about 3700 mg, about 1900 mg to about 3600 mg, about 1900 mg to about 3500 mg, about 1900 mg to about 3400 mg, about 1900 mg to about 3300 mg, about 1900 mg to about 3200 mg, about 1900 mg to about 3100 mg, about 1900 mg to about 3000 mg, about 1900 mg to about 2900 mg, about 1900 mg to about 2800 mg, about 1900 mg to about 2700 mg, about 1900 mg to about 2600 mg, about 1900 mg to about 2500 mg, about 1900 mg to about 2400 mg, about 1900 mg to about 2300 mg, about 1900 mg to about 2200 mg, about 1900 mg to about 2100 mg, about 1900 mg to about 2000 mg, about 2000 mg to about 4000 mg, about 2000 mg to about 3900 mg, about 2000 mg to about 3800 mg, about 2000 mg to about 3700 mg, about 2000 mg to about 3600 mg, about 2000 mg to about 3500 mg, about 2000 mg to about 3400 mg, about 2000 mg to about 3300 mg, about 2000 mg to about 3200 mg, about 2000 mg to about 3100 mg, about 2000 mg to about 3000 mg, about 2000 mg to about 2900 mg, about 2000 mg to about 2800 mg, about 2000 mg to about 2700 mg, about 2000 mg to about 2600 mg, about 2000 mg to about 2500 mg, about 2000 mg to about 2400 mg, about 2000 mg to about 2300 mg, about 2000 mg to about 2200 mg, about 2000 mg to about 2100 mg, about 2100 mg to about 4000 mg, about 2100 mg to about 3900 mg, about 2100 mg to about 3800 mg, about 2100 mg to about 3700 mg, about 2100 mg to about 3600 mg, about 2100 mg to about 3500 mg, about 2100 mg to about 3400 mg, about 2100 mg to about 3300 mg, about 2100 mg to about 3200 mg, about 2100 mg to about 3100 mg, about 2100 mg to about 3000 mg, about 2100 mg to about 2900 mg, about 2100 mg to about 2800 mg, about 2100 mg to about 2700 mg, about 2100 mg to about 2600 mg, about 2100 mg to about 2500 mg, about 2100 mg to about 2400 mg, about 2100 mg to about 2300 mg, about 2100 mg to about 2200 mg, about 2200 mg to about 4000 mg, about 2200 mg to about 3900 mg, about 2200 mg to about 3800 mg, about 2200 mg to about 3700 mg, about 2200 mg to about 3600 mg, about 2200 mg to about 3500 mg, about 2200 mg to about 3400 mg, about 2200 mg to about 3300 mg, about 2200 mg to about 3200 mg, about 2200 mg to about 3100 mg, about 2200 mg to about 3000 mg, about 2200 mg to about 2900 mg, about 2200 mg to about 2800 mg, about 2200 mg to about 2700 mg, about 2200 mg to about 2600 mg, about 2200 mg to about 2500 mg, about 2200 mg to about 2400 mg, about 2200 mg to about 2300 mg, about 2300 mg to about 4000 mg, about 2300 mg to about 3900 mg, about 2300 mg to about 3800 mg, about 2300 mg to about 3700 mg, about 2300 mg to about 3600 mg, about 2300 mg to about 3500 mg, about 2300 mg to about 3400 mg, about 2300 mg to about 3300 mg, about 2300 mg to about 3200 mg, about 2300 mg to about 3100 mg, about 2300 mg to about 3000 mg, about 2300 mg to about 2900 mg, about 2300 mg to about 2800 mg, about 2300 mg to about 2700 mg, about 2300 mg to about 2600 mg, about 2300 mg to about 2500 mg, about 2300 mg to about 2400 mg, about 2400 mg to about 4000 mg, about 2400 mg to about 3900 mg, about 2400 mg to about 3800 mg, about 2400 mg to about 3700 mg, about 2400 mg to about 3600 mg, about 2400 mg to about 3500 mg, about 2400 mg to about 3400 mg, about 2400 mg to about 3300 mg, about 2400 mg to about 3200 mg, about 2400 mg to about 3100 mg, about 2400 mg to about 3000 mg, about 2400 mg to about 2900 mg, about 2400 mg to about 2800 mg, about 2400 mg to about 2700 mg, about 2400 mg to about 2600 mg, about 2400 mg to about 2500 mg, about 2500 mg to about 4000 mg, about 2500 mg to about 3900 mg, about 2500 mg to about 3800 mg, about 2500 mg to about 3700 mg, about 2500 mg to about 3600 mg, about 2500 mg to about 3500 mg, about 2500 mg to about 3400 mg, about 2500 mg to about 3300 mg, about 2500 mg to about 3200 mg, about 2500 mg to about 3100 mg, about 2500 mg to about 3000 mg, about 2500 mg to about 2900 mg, about 2500 mg to about 2800 mg, about 2500 mg to about 2700 mg, about 2500 mg to about 2600 mg, about 2600 mg to about 4000 mg, about 2600 mg to about 3900 mg, about 2600 mg to about 3800 mg, about 2600 mg to about 3700 mg, about 2600 mg to about 3600 mg, about 2600 mg to about 3500 mg, about 2600 mg to about 3400 mg, about 2600 mg to about 3300 mg, about 2600 mg to about 3200 mg, about 2600 mg to about 3100 mg, about 2600 mg to about 3000 mg, about 2600 mg to about 2900 mg, about 2600 mg to about 2800 mg, about 2600 mg to about 2700 mg, about 2700 mg to about 4000 mg, about 2700 mg to about 3900 mg, about 2700 mg to about 3800 mg, about 2700 mg to about 3700 mg, about 2700 mg to about 3600 mg, about 2700 mg to about 3500 mg, about 2700 mg to about 3400 mg, about 2700 mg to about 3300 mg, about 2700 mg to about 3200 mg, about 2700 mg to about 3100 mg, about 2700 mg to about 3000 mg, about 2700 mg to about 2900 mg, about 2700 mg to about 2800 mg, about 2800 mg to about 4000 mg, about 2800 mg to about 3900 mg, about 2800 mg to about 3800 mg, about 2800 mg to about 3700 mg, about 2800 mg to about 3600 mg, about 2800 mg to about 3500 mg, about 2800 mg to about 3400 mg, about 2800 mg to about 3300 mg, about 2800 mg to about 3200 mg, about 2800 mg to about 3100 mg, about 2800 mg to about 3000 mg, about 2800 mg to about 2900 mg, about 2900 mg to about 4000 mg, about 2900 mg to about 3900 mg, about 2900 mg to about 3800 mg, about 2900 mg to about 3700 mg, about 2900 mg to about 3600 mg, about 2900 mg to about 3500 mg, about 2900 mg to about 3400 mg, about 2900 mg to about 3300 mg, about 2900 mg to about 3200 mg, about 2900 mg to about 3100 mg, about 2900 mg to about 3000 mg, about 3000 mg to about 4000 mg, about 3000 mg to about 3900 mg, about 3000 mg to about 3800 mg, about 3000 mg to about 3700 mg, about 3000 mg to about 3600 mg, about 3000 mg to about 3500 mg, about 3000 mg to about 3400 mg, about 3000 mg to about 3300 mg, about 3000 mg to about 3200 mg, about 3000 mg to about 3100 mg, about 3100 mg to about 4000 mg, about 3100 mg to about 3900 mg, about 3100 mg to about 3800 mg, about 3100 mg to about 3700 mg, about 3100 mg to about 3600 mg, about 3100 mg to about 3500 mg, about 3100 mg to about 3400 mg, about 3100 mg to about 3300 mg, about 3100 mg to about 3200 mg, about 3200 mg to about 4000 mg, about 3200 mg to about 3900 mg, about 3200 mg to about 3800 mg, about 3200 mg to about 3700 mg, about 3200 mg to about 3600 mg, about 3200 mg to about 3500 mg, about 3200 mg to about 3400 mg, about 3200 mg to about 3300 mg, about 3300 mg to about 4000 mg, about 3300 mg to about 3900 mg, about 3300 mg to about 3800 mg, about 3300 mg to about 3700 mg, about 3300 mg to about 3600 mg, about 3300 mg to about 3500 mg, about 3300 mg to about 3400 mg, about 3400 mg to about 4000 mg, about 3400 mg to about 3900 mg, about 3400 mg to about 3800 mg, about 3400 mg to about 3700 mg, about 3400 mg to about 3600 mg, about 3400 mg to about 3500 mg, about 3500 mg to about 4000 mg, about 3500 mg to about 3900 mg, about 3500 mg to about 3800 mg, about 3500 mg to about 3700 mg, about 3500 mg to about 3600 mg, about 3600 mg to about 4000 mg, about 3600 mg to about 3900 mg, about 3600 mg to about 3800 mg, about 3600 mg to about 3700 mg, about 3700 mg to about 4000 mg, about 3700 mg to about 3900 mg, about 3700 mg to about 3800 mg, about 3800 mg to about 4000 mg, about 3800 mg to about 3900 mg, or about 3900 mg to about 4000 mg.

In one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody selected from 300 mg, 450 mg, 750 mg, 900 mg, 1350 mg, 1800 mg, or 3600 mg. The C1D1 may be administered, for example, intravenously (IV) or subcutaneously (SC) (e.g., by a pump (e.g., by a patch pump).

In some aspects, any of the doses disclosed herein may be administered IV. Any suitable approach for IV administration may be used, including injection (e.g., a bolus injection) or infusion. In some examples, the anti-tryptase antibody may be administered IV by infusion. For example, the IV infusion may use pressure supplied by gravity (e.g., a drip) or using a pump (e.g., an infusion pump). In some examples, the IV infusion may be continuous or intermittent. In some examples, a central venous catheter, a peripheral venous catheter, a peripherally inserted central catheter (PICC), a midline catheter, or an implantable port may be used for IV administration. In some examples, the anti-tryptase antibody may be administered IV using a pump. Any suitable pump may be used for IV administration, for example, an infusion pump (e.g., an ambulatory infusion pump or a stationary infusion pump), a syringe pump, a patch pump, or a large volume pump (LVP).

In other aspects, any of the doses disclosed herein may be administered SC. Any suitable approach for SC administration may be used, including injection (e.g., a bolus injection) or infusion. For example, the anti-tryptase antibody may be administered SC using a pump (e.g., a patch pump, a syringe pump (e.g., a syringe pump with an infusion set), or an infusion pump (e.g., an ambulatory infusion pump or a stationary infusion pump)), a pre-filled syringe, a pen injector, or an autoinjector.

For example, in any of the methods or uses disclosed herein, the anti-tryptase antibody may be administered SC using a pump. In some examples, a pump may be used for patient or health care provider (HCP) convenience, an improved safety profile (e.g., in terms of a drug's mechanism of action or the risk of IV-related infection), and/or for a combination therapy. Any suitable pump may be used, e.g., a patch pump, a syringe pump (e.g., a syringe pump with an infusion set), an infusion pump (e.g., an ambulatory infusion pump or a stationary infusion pump), or an LVP. In particular examples, the anti-tryptase antibody may be administered SC using a patch pump. In some examples, the pump (e.g., the patch pump) may be a wearable or on-body pump (e.g., a wearable or on-body patch pump), for example, an Enable ENFUSE® on-body infusor or a West SMARTDOSE® wearable injector (e.g., a West SMARTDOSE® 10 wearable injector). In other examples, the anti-tryptase antibody may be administered SC using a syringe pump (e.g., a syringe pump with an infusion set).

For example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 300 mg. In some aspects, the C1D1 is administered IV. In other aspects, the C1D1 is administered SC (e.g., by a pump (e.g., by a patch pump).

In another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 450 mg. In some aspects, the C1D1 is administered IV. In other aspects, the C1D1 is administered SC (e.g., by a pump (e.g., by a patch pump).

In yet another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 750 mg. In some aspects, the C1D1 is administered IV. In other aspects, the C1D1 is administered SC (e.g., by a pump (e.g., by a patch pump).

In a further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 900 mg. In some aspects, the C1D1 is administered IV. In other aspects, the C1D1 is administered SC (e.g., by a pump (e.g., by a patch pump).

In yet a further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 1350 mg. In some aspects, the C1D1 is administered IV. In other aspects, the C1D1 is administered SC (e.g., by a pump (e.g., by a patch pump).

In a still further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 1800 mg. In some aspects, the C1D1 is administered IV. In other aspects, the C1D1 is administered SC (e.g., by a pump (e.g., by a patch pump).

In another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ) of the anti-tryptase antibody of 3600 mg. In some aspects, the C1D1 is administered IV. In other aspects, the C1D1 is administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody selected from 300 mg, 450 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump).

For example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 300 mg. In some aspects, the C1D1 is administered IV. In other aspects, the C1D1 is administered SC (e.g., by a pump (e.g., by a patch pump).

In another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 450 mg. In some aspects, the C1D1 is administered IV. In other aspects, the C1D1 is administered SC (e.g., by a pump (e.g., by a patch pump).

In yet another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 750 mg. In some aspects, the C1D1 is administered IV. In other aspects, the C1D1 is administered SC (e.g., by a pump (e.g., by a patch pump).

In a further example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 900 mg. In some aspects, the C1D1 is administered IV. In other aspects, the C1D1 is administered SC (e.g., by a pump (e.g., by a patch pump).

In yet a further example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 1350 mg. In some aspects, the C1D1 is administered IV. In other aspects, the C1D1 is administered SC (e.g., by a pump (e.g., by a patch pump).

In a still further example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 1800 mg. In some aspects, the C1D1 is administered IV. In other aspects, the C1D1 is administered SC (e.g., by a pump (e.g., by a patch pump).

In another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 3600 mg. In some aspects, the C1D1 is administered IV. In other aspects, the C1D1 is administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody selected from 300 mg, 450 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump).

For example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 300 mg. In some aspects, the C1D1 is administered IV. In other aspects, the C1D1 is administered SC (e.g., by a pump (e.g., by a patch pump).

In another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 450 mg. In some aspects, the C1D1 is administered IV. In other aspects, the C1D1 is administered SC (e.g., by a pump (e.g., by a patch pump).

In yet another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 750 mg. In some aspects, the C1D1 is administered IV. In other aspects, the C1D1 is administered SC (e.g., by a pump (e.g., by a patch pump).

In a further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 900 mg. In some aspects, the C1D1 is administered IV. In other aspects, the C1D1 is administered SC (e.g., by a pump (e.g., by a patch pump).

In yet a further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 1350 mg. In some aspects, the C1D1 is administered IV. In other aspects, the C1D1 is administered SC (e.g., by a pump (e.g., by a patch pump).

In a still further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 1800 mg. In some aspects, the C1D1 is administered IV. In other aspects, the C1D1 is administered SC (e.g., by a pump (e.g., by a patch pump).

In another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase antibody of 3600 mg. In some aspects, the C1D1 is administered IV. In other aspects, the C1D1 is administered SC (e.g., by a pump (e.g., by a patch pump).

In any of the aspects disclosed herein, the dosing cycle may further include one or more additional doses of the anti-tryptase antibody. The dosing cycle may include any suitable number of additional doses (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more additional doses) of the anti-tryptase antibody. For example, in some aspects, the dosing cycle may include a second dose (C1D2). In another example, in some aspects, the dosing cycle may include a C1D2 and a third dose (C1D3). The one or more additional doses may be equal to or unequal to the C1D1. For example, in some aspects, the dosing cycle includes a second dose (C1D2) and a third dose (C1D3) of the anti-tryptase antibody, wherein the C1D2 and the C1D3 are each equal to the C1D1. The one or more additional doses may be administered by any suitable administration route, e.g., IV or SC (e.g., by a pump (e.g., by a patch pump).

For example, in one aspect, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are selected from 300 mg, 450 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

For example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump).

In a further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet a further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump).

In a still further example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1 D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is a method of treating a patient having asthma, the method including administering to a patient having asthma an anti-tryptase antibody (e.g., an anti-tryptase beta antibody) in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

In another aspect, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are selected from 300 mg, 450 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

For example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ) of the anti-tryptase antibody of 750 mg. In some aspects, the C1D1, the C1D2, and the C1D3 are administered IV. In other aspects, the C1D1, the C1D2, and the C1D3 are administered SC (e.g., by a pump (e.g., by a patch pump).

In a further example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet a further example, provided herein is an anti-tryptase antibody (e.g., an anti-tryptase beta antibody) antibody for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump).

In a still further example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is an anti-tryptase antibody (e.g., an anti-tryptase beta antibody) for use in treating a patient having asthma, wherein the anti-tryptase antibody is for administration to a patient having asthma in a dosing regimen including a dosing cycle, wherein the dosing cycle includes a first dose (C1D1 ), a second dose (C1D2), and a third dose (C1D3) of the anti- tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

In another aspect, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are selected from 300 mg, 450 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

For example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump).

In a further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet a further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump).

In a still further example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is the use of an anti-tryptase antibody (e.g., an anti-tryptase beta antibody) in the manufacture of a medicament for treating a patient having asthma, wherein the medicament is for administration to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

The doses of each dosing cycle may be administered to the subject at any suitable time interval. For example, in some aspects, the doses of the dosing cycle are administered to the subject every four weeks (q4w).

For example, in one aspect, provided herein is a method of treating a patient having asthma, the method including administering to a patient having asthma an anti-tryptase antibody (e.g., an anti-tryptase beta antibody) at a dose of 300 mg IV, 450 mg IV, 750 mg SC, 900 mg IV, 1350 mg IV, 1800 mg IV, or 3600 mg IV every four weeks (q4w).

For example, provided herein is a method of treating a patient having asthma, the method including administering to a patient having asthma an anti-tryptase antibody (e.g., an anti-tryptase beta antibody) at a dose of 300 mg IV every four weeks (q4w). In another example, provided herein is a method of treating a patient having asthma, the method including administering to a patient having asthma an anti-tryptase antibody (e.g., an anti-tryptase beta antibody) at a dose of 450 mg IV every four weeks (q4w).

In yet another example, provided herein is a method of treating a patient having asthma, the method including administering to a patient having asthma an anti-tryptase antibody (e.g., an anti-tryptase beta antibody) at a dose of 750 mg SC (e.g., by a pump (e.g., by a patch pump) every four weeks (q4w).

In a further example, provided herein is a method of treating a patient having asthma, the method including administering to a patient having asthma an anti-tryptase antibody (e.g., an anti-tryptase beta antibody) at a dose of 900 mg IV every four weeks (q4w).

In yet a further example, provided herein is a method of treating a patient having asthma, the method including administering to a patient having asthma an anti-tryptase antibody (e.g., an anti-tryptase beta antibody) at a dose of 1350 mg IV every four weeks (q4w).

In a still further example, provided herein is a method of treating a patient having asthma, the method including administering to a patient having asthma an anti-tryptase antibody (e.g., an anti-tryptase beta antibody) at a dose of 1800 mg IV every four weeks (q4w).

In another example, provided herein is a method of treating a patient having asthma, the method including administering to a patient having asthma an anti-tryptase antibody (e.g., an anti-tryptase beta antibody) at a dose of 3600 mg IV every four weeks (q4w).

In another aspect, provided herein is an anti-tryptase antibody (e.g., an anti-tryptase beta antibody) for use in treating a patient having asthma, wherein the anti-tryptase antibody is for administration to a patient having asthma at a dose selected from 300 mg IV, 450 mg IV, 750 mg SC (e.g., by a pump (e.g., by a patch pump), 900 mg IV, 1350 mg IV, 1800 mg IV, or 3600 mg IV every four weeks (q4w).

For example, provided herein is an anti-tryptase antibody (e.g., an anti-tryptase beta antibody) for use in treating a patient having asthma, wherein the anti-tryptase antibody is for administration to a patient having asthma at a dose of 300 mg IV every four weeks (q4w).

In another example, provided herein is an anti-tryptase antibody (e.g., an anti-tryptase beta antibody) for use in treating a patient having asthma, wherein the anti-tryptase antibody is for administration to a patient having asthma at a dose of 450 mg IV every four weeks (q4w).

In yet another example, provided herein is an anti-tryptase antibody (e.g., an anti-tryptase beta antibody) for use in treating a patient having asthma, wherein the anti-tryptase antibody is for administration to a patient having asthma at a dose of 750 mg SC (e.g., by a pump (e.g., by a patch pump) every four weeks (q4w).

In a further example, provided herein is an anti-tryptase antibody (e.g., an anti-tryptase beta antibody) for use in treating a patient having asthma, wherein the anti-tryptase antibody is for administration to a patient having asthma at a dose of 900 mg IV every four weeks (q4w).

In yet a further example, provided herein is an anti-tryptase antibody (e.g., an anti-tryptase beta antibody) for use in treating a patient having asthma, wherein the anti-tryptase antibody is for administration to a patient having asthma at a dose of 1350 mg IV every four weeks (q4w).

In a still further example, provided herein is an anti-tryptase antibody (e.g., an anti-tryptase beta antibody) antibody for use in treating a patient having asthma, wherein the anti-tryptase antibody is for administration to a patient having asthma at a dose of 1800 mg IV every four weeks (q4w).

In another example, provided herein is an anti-tryptase antibody (e.g., an anti-tryptase beta antibody) for use in treating a patient having asthma, wherein the anti-tryptase antibody is for administration to a patient having asthma at a dose of 3600 mg IV every four weeks (q4w).

In another aspect, provided herein is the use of an anti-tryptase antibody (e.g., an anti-tryptase beta antibody) antibody in the manufacture of a medicament for treating a patient having asthma, wherein the medicament is for administration to a patient having asthma at a dose selected from 300 mg IV, 450 mg IV, 750 mg SC (e.g., by a pump (e.g., by a patch pump), 900 mg IV, 1350 mg IV, 1800 mg IV, or 3600 mg IV every four weeks (q4w).

For example, provided herein is the use of an anti-tryptase antibody (e.g., an anti-tryptase beta antibody) in the manufacture of a medicament for treating a patient having asthma, wherein the medicament is for administration to a patient having asthma at a dose of 300 mg IV every four weeks (q4w).

In another example, provided herein is the use of an anti-tryptase antibody (e.g., an anti-tryptase beta antibody) in the manufacture of a medicament for treating a patient having asthma, wherein the medicament is for administration to a patient having asthma at a dose of 450 mg IV every four weeks (q4w).

In yet another example, provided herein is the use of an anti-tryptase antibody (e.g., an anti-tryptase beta antibody) in the manufacture of a medicament for treating a patient having asthma, wherein the medicament is for administration to a patient having asthma at a dose of 750 mg SC (e.g., by a pump (e.g., by a patch pump) every four weeks (q4w).

In a further example, provided herein is the use of an anti-tryptase antibody (e.g., an anti-tryptase beta antibody) in the manufacture of a medicament for treating a patient having asthma, wherein the medicament is for administration to a patient having asthma at a dose of 900 mg IV every four weeks (q4w).

In yet a further example, provided herein is the use of an anti-tryptase antibody (e.g., an anti-tryptase beta antibody) in the manufacture of a medicament for treating a patient having asthma, wherein the medicament is for administration to a patient having asthma at a dose of 1350 mg IV every four weeks (q4w).

In a still further example, provided herein is the use of an anti-tryptase antibody (e.g., an anti-tryptase beta antibody) in the manufacture of a medicament for treating a patient having asthma, wherein the medicament is for administration to a patient having asthma at a dose of 1800 mg IV every four weeks (q4w).

In another example, provided herein is the use of an anti-tryptase antibody (e.g., an anti-tryptase beta antibody) in the manufacture of a medicament for treating a patient having asthma, wherein the medicament is for administration to a patient having asthma at a dose of 3600 mg IV every four weeks (q4w).

Each dosing cycle may have any suitable length.

For example, in some aspects, each dosing cycle may have a length of about 57 days.

The doses of each dosing cycle may be administered on any suitable day(s) of the dosing cycle. For example, in some aspects, the C1D1 is administered on Day 1 of the dosing cycle, the C1D2 is administered on Day 29 of the dosing cycle, and the C1D3 is administered on Day 57 of the dosing cycle.

In other aspects, the dosing cycle may have a length of about 48 weeks. For example, in some aspects, the doses of the dosing cycle are administered every four weeks (q4w) for 48 weeks. For example, in some aspects, the C1D1 is administered on Week 0 of the dosing cycle, a C1D2 is administered on Week 4 of the dosing cycle, a C1D3 is administered on Week 8 of the dosing cycle, a C1D3 is administered on Week 12 of the dosing cycle, a C1D4 is administered on Week 16 of the dosing cycle, a C1D5 is administered on Week 20 of the dosing cycle, a C1D6 is administered on Week 24 of the dosing cycle, a C1D7 is administered on Week 28 of the dosing cycle, a C1D8 is administered on Week 32 of the dosing cycle, a C1D9 is administered on Week 36 of the dosing cycle, a C1D10 is administered on Week 40 of the dosing cycle, a C1D11 is administered on Week 44 of the dosing cycle, and a C1D12 is administered on Week 48 of the dosing cycle.

The dosing regimens described herein may include any suitable number of dosing cycles. For example, in some aspects, the dosing regimen includes or consists of one dosing cycle. In other aspects, the dosing regimen may include more than one dosing cycle (e.g., 2, 3, 4, 5, 6, 7, 8, 9, 10, 11, 12, 13, 14, 15, 16, 17, 18, 19, 20, or more dosing cycles).

The methods, compositions for use (e.g., anti-tryptase antibodies for use), and uses of the present disclosure may be used for treating any suitable type of asthma. For example, in some aspects, the asthma is moderate asthma. In some aspects, the moderate asthma is uncontrolled despite standard-of-care therapy. In some aspects, the asthma is severe asthma. In some aspects, the severe asthma is uncontrolled despite standard-of-care therapy. In other aspects, the asthma is allergic asthma. In other aspects, the asthma is atopic asthma.

In some aspects, the patient is receiving inhaled corticosteroid therapy and/or a controller medication. In some aspects, the patient is receiving inhaled corticosteroid therapy. In some aspects, the patient is receiving a controller medication. In some aspects, the patient is receiving inhaled corticosteroid therapy and a controller medication.

Any suitable inhaled corticosteroid (e.g., fluticasone, mudesonide, mometasone, flunisolide, beclomethasone, or ciclesonide) and/or controller (e.g., a long-acting β-agonist (LABA), a leukotriene modulator (e.g., a leukotriene modifier (LTM) or a leukotriene receptor antagonist (LTRA)), a long-acting muscarinic antagonist (LAMA), a long-acting theophylline preparation, or a combination thereof) may be used. In some aspects, the patient is receiving daily inhaled corticosteroid therapy and at least one of the following controller medications: an LABA (e.g., salmeterol, formoterol, or a combination of a LABA and an inhaled corticosteroid (e.g., fluticasone and salmeterol, budesonide and formoterol, moetasone and formoterol, or fluticasone and vilanterol)), a leukotriene modulator (e.g., an LTM (e.g., montelukast sodium, zafirlukast, or zileuton) or an LTRA (e.g., montelukast or zafirlukast)), an LAMA (e.g., aclidinium, glycopyrronium, tiotropium, or umeclidinium), or a long-acting theophylline preparation.

In some aspects, the inhaled corticosteroid is 100 μg of fluticasone propionate or an equivalent.

In some aspects, the patient is receiving allergen immunotherapy.

In some aspects, the patient is receiving maintenance oral corticosteroids (e.g., daily or every other day)

In some aspects, the patient is receiving systemic corticosteroids (e.g., oral, IV, or IM systemic corticosteroids).

In some aspects, the patient is receiving bronchial thermoplasty.

In some aspects, the patient is receiving bilevel positive airway pressure therapy.

In some aspects, the patient is receiving mast cell stabilizers (e.g., chromolyn).

Any suitable anti-tryptase antibody (e.g., anti-tryptase beta antibody) may be used in any of the aspects described herein. For example, any of the anti-tryptase antibodies described in Section IV, Subsection A below can be used. In some aspects, the anti-tryptase antibody may be any anti-tryptase antibody described in International Patent Application Publication No. WO 2018/148585, which is incorporated herein by reference in its entirety.

For example, any of the anti-tryptase (e.g., anti-tryptase beta) antibodies may include one, two, three, four, five, or all six of the following complementarity determining regions (CDRs): (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

For example, in one aspect, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody selected from 300 mg IV, 450 mg IV, 750 mg SC, 900 mg IV, 1350 mg IV, 1800 mg IV, or 3600 mg IV, wherein the anti-tryptase beta antibody includes one, two, three, four, five, or all six of the following complementarity determining regions (CDRs): (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

For example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 300 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 450 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet another example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 750 mg SC, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a further example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 900 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet a further example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 1350 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a still further example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 1800 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is a method of treating a patient having asthma, the method including administering to a patient having asthma an anti-tryptase beta antibody in a dosing regimen including a dosing cycle, wherein the dosing cycle includes a first dose (C1D1 ) of the anti-tryptase beta antibody of 3600 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another aspect, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody selected from 300 mg IV, 450 mg IV, 750 mg SC (e.g., by a pump (e.g., by a patch pump), 900 mg IV, 1350 mg IV, 1800 mg IV, or 3600 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

For example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 300 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 450 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet another example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 750 mg SC (e.g., by a pump (e.g., by a patch pump), wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a further example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 900 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet a further example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 1350 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a still further example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 1800 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is an anti-tryptase beta antibody for use in treating a patient having asthma, wherein the anti-tryptase beta antibody is for administration to a patient having asthma in a dosing regimen including a dosing cycle, wherein the dosing cycle includes a first dose (C1D1 ) of the anti-tryptase beta antibody of 3600 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another aspect, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody selected from 300 mg IV, 450 mg IV, 750 mg SC (e.g., by a pump (e.g., by a patch pump), 900 mg IV, 1350 mg IV, 1800 mg IV, or 3600 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

For example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 300 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody 450 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet another example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 750 mg SC (e.g., by a pump (e.g., by a patch pump), wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a further example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 900 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In yet a further example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 1350 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In a still further example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 1800 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is the use of an anti-tryptase beta antibody in the manufacture of a medicament for treating a patient having asthma, wherein the medicament is for administration to a patient having asthma in a dosing regimen including a dosing cycle, wherein the dosing cycle includes a first dose (C1D1 ) of the anti-tryptase beta antibody of 3600 mg IV, wherein the anti-tryptase beta antibody includes one, two, three, four, five, or all six of the following CDRs: (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In another example, provided herein is a method of treating a patient having asthma, the method comprising administering to a patient having asthma an anti-tryptase beta antibody in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In yet another example, provided herein is an anti-tryptase beta antibody for use in treating a patient having asthma, wherein the anti-tryptase beta antibody is for administration to a patient having asthma in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In a further example, provided herein is the use of an anti-tryptase beta antibody in the manufacture of a medicament for treating a patient having asthma, wherein the anti-tryptase beta antibody is for administration to a patient having asthma in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In any of the aspects provided herein, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7; (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8; or (c) a VH domain as in (a) and a VL domain as in (b).

For example, in some aspects, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7. In some aspects, the VH domain includes the amino acid sequence of SEQ ID NO: 7.

In another example, in some aspects, the antibody may include (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8. In some aspects, the VL domain includes the amino acid sequence of SEQ ID NO: 8.

In any of the aspects described herein, the VH domain may include the amino acid sequence of SEQ ID NO: 7 and the VL domain includes the amino acid sequence of SEQ ID NO: 8.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 9 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 9 and (b) a light chain including the amino acid sequence of SEQ ID NO: 10.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 11 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 11 and (b) a light chain including the amino acid sequence of SEQ ID NO: 10.

Any of the aspects disclosed herein may include administering one or more additional therapeutic agents to the patient. The one or more additional therapeutic agents may be standard of care for asthma. Any suitable standard of care for asthma may be used, e.g., inhaled corticosteroids, long-acting beta agonists, and other controller medications. A person of skill in the art will be able to select a suitable standard of care as appropriate. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. The combined administration includes co-administration, using separate formulations or a single pharmaceutical formulation, and consecutive administration in either order, wherein preferably there is a time period while both (or all) active agents simultaneously exert their biological activities. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. When administered sequentially, the combination may be administered in two or more administrations.

Such combination therapies noted above encompass combined administration (where two or more therapeutic agents are included in the same or separate formulations), and separate administration, in which case, administration of an agent (e.g., an anti-tryptase antibody), or a pharmaceutical composition thereof, can occur prior to, simultaneously, and/or following, administration of the additional therapeutic agent(s). In one aspect, administration of an agent (e.g., an anti-tryptase antibody), or a pharmaceutical composition thereof, and administration of an additional therapeutic agent occur within about one month; or within about one, two, or three weeks; or within about one, two, three, four, five, or six days; or within about 1, 2, 3, 4, 5, 6, 7, 8, or 9 hours; or within about 1, 5, 10, 20, 30, 40, or 50 minutes, of each other. For aspects involving sequential administration, the agent (e.g., an anti-tryptase antibody) may be administered prior to or after administration of the additional therapeutic agent(s).

In any of the aspects described herein, the anti-tryptase antibody, and any additional therapeutic agent, can be administered by any suitable means, including parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The administration may be systemic or local. In addition, the antagonist may suitably be administered by pulse infusion, e.g., with declining doses of the antagonist.

In any of the aspects described herein, the anti-tryptase antibody, and any additional therapeutic agent, can be administered intravenously.

In any of the aspects described herein, the anti-tryptase antibody, and any additional therapeutic agent, can be administered subcutaneously (e.g., by a pump (e.g., by a patch pump)).

Any therapeutic agent, e.g., an anti-tryptase antibody, any additional therapeutic agent, or pharmaceutical compositions thereof, would be formulated, dosed, and administered in a fashion consistent with good medical practice. Dosages for anti-tryptase antibodies are disclosed herein. Dosages for additional therapeutic agents are known in the art. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent (e.g., an anti-tryptase antibody), or pharmaceutical composition thereof, need not be, but is optionally formulated with one or more agents currently used to prevent or treat the disorder in question (e.g., asthma). The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

As one example, for the prevention or treatment of disease, the appropriate dosage of an antibody, when used alone or in combination with one or more other additional therapeutic agents, will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg to 10 mg/kg) of antibody can be an initial candidate dosage for administration to the patient, whether, for example, by one or more separate administrations, or by continuous infusion. One typical daily dosage might range from about 1 μg/kg to 200 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. For example, a dose may be administered once per month. An initial higher loading dose, followed by one or more lower doses, may be administered. However, other dosage regimens may be useful. The progress of this therapy is easily monitored by conventional techniques and assays. In some aspects, a dose of about 50 mg/mL to about 200 mg/mL (e.g., about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL of an antibody may be administered.

### IV. Compositions and Pharmaceutical Formulations

Any suitable composition (e.g., anti-tryptase antibody) or a pharmaceutical formulation thereof can be used in the methods, compositions for use, and uses described herein. Non-limiting examples suitable for the methods, compositions for use, and uses described herein are described further below.

A. Anti-Tryptase Antibodies

Any suitable anti-tryptase antibody can be used in the methods, compositions for use, and uses of the invention. For example, the anti-tryptase antibody may be any anti-tryptase antibody described in International Patent Application Publication No. WO 2018/148585.

In some aspects, the anti-tryptase antibody (e.g., the anti-tryptase beta antibody) can include at least one, at least two, at least three, at least four, at least five, or all six CDRs selected from (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 1-6. For example, in some aspects, the anti-tryptase antibody includes (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In some aspects, the anti-tryptase antibody (e.g., the anti-tryptase beta antibody) can include (a) a heavy chain variable (VH) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 7; (b) a light chain variable (VL) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 8; or (c) a VH domain as in (a) and a VL domain as in (b). For example, in some aspects, the VH domain comprises the amino acid sequence of SEQ ID NO: 7. In some aspects, the VL domain comprises the amino acid sequence of SEQ ID NO: 8. In particular aspects, the VH domain comprises the amino acid sequence of SEQ ID NO: 7 and the VL domain comprises the amino acid sequence of SEQ ID NO: 8.

In some aspects, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 9 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some aspects, the anti-tryptase antibody (e.g., the anti-tryptase beta antibody) includes (a) a heavy chain comprising the amino acid sequence of SEQ ID NO: 9 and (b) a light chain comprising the amino acid sequence of SEQ ID NO: 10.

In other aspects, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 11 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some aspects, the anti-tryptase antibody (e.g., the anti-tryptase beta antibody) includes (a) a heavy chain comprising the amino acid sequence of SEQ ID NO: 11 and (b) a light chain comprising the amino acid sequence of SEQ ID NO: 10.

In some aspects, the anti-tryptase antibody is an antibody that binds to the same epitope as any one of the preceding antibodies. In some aspects, whether the antibody binds to the same epitope or competes for binding to human tryptase beta 1 is determined by an epitope binning assay. In some aspects, the epitope binning assay is an OCTET® epitope binning assay such as described in Example 3, Section C of WO 2018/148585. In some aspects, human tryptase beta 1 monomer protein is biotinylated at Lys residue by reacting with NHS-PEG4-biotin. Biotinylated monomer is diluted to 5 μg/ml in kinetics buffer (ForteBio, Inc.) and immobilized onto streptavidin sensor tips (ForteBio, Inc.). After the immobilization step, human tryptase beta 1-immobilized sensors are saturated with the first antibody, diluted at 10-20 82 g/ml, followed by binding with second antibody diluted at 2.5 μg/ml. In some aspects, the epitope binning assay is performed at 30° C.

In some aspects, the anti-tryptase antibody is an antibody that competes for binding with, or cross-blocks or is cross-blocked by any one of the preceding antibodies.

It is expressly contemplated that any such anti-tryptase antibodies for use in any of the aspects enumerated herein may have any of the features, singly or in combination, described in Sections 1-7 below.

1. Antibody Affinity

In certain aspects, an antibody provided herein (e.g., an anti-tryptase antibody) has a dissociation constant (K_{D}) of ≤1 μM, ≤100 nM, ≤10 nM, ≤1 nM, ≤0.1 nM, ≤0.01 nM, ≤1 pM, or ≤0.1 pM (e.g., 10⁻⁶ M or less, e.g., from 10⁻⁶ M to 10⁻⁹M or less, e.g., from 10⁻⁹M to 10⁻¹³ M or less). For example, in some aspects, an anti-tryptase antibody binds to tryptase (e.g., human tryptase, e.g., human tryptase beta) with a K_{D} of about 100 nM or lower (e.g., 100 nM or lower, 10 nM or lower, 1 nM or lower, 100 pM or lower, 10 pM or lower, 1 pM or lower, or 0.1 pM or lower). In some aspects, the antibody binds tryptase (e.g., human tryptase, e.g., human tryptase beta) with a K_{D} of 10 nM or lower (e.g., 10 nM or lower, 1 nm or lower, 100 pM or lower, 10 pM or lower, 1 pM or lower, or 0.1 pM or lower). In some aspects, the antibody binds tryptase (e.g., human tryptase, e.g., human tryptase beta) with a K_{D} of 1 nM or lower (e.g., 1 nm or lower, 100 pM or lower, 10 pM or lower, 1 pM or lower, or 0.1 pM or lower). In some aspects, any of the anti-tryptase antibodies described above or herein binds to tryptase (e.g., human tryptase, e.g., human tryptase beta) with a K_{D} of about 0.5 nM or lower (e.g., 0.5 nm or lower, 400 pM or lower, 300 pM or lower, 200 pM or lower, 100 pM or lower, 50 pM or lower, 25 pM or lower, 10 pM or lower, 1 pM or lower, or 0.1 pM or lower). In some aspects, the antibody binds tryptase (e.g., human tryptase, e.g., human tryptase beta) with a K_{D} between about 0.1 nM to about 0.5 nM (e.g., about 0.1 nM, about 0.2 nM, about 0.3 nM, about 0.4 nM, or about 0.5 nM). In some aspects, the antibody binds tryptase (e.g., human tryptase, e.g., human tryptase beta) with a K_{D} of about 0.4 nM. In some aspects, the antibody binds tryptase (e.g., human tryptase, e.g., human tryptase beta) with a K_{D} of about 0.18 nM. Any of the other antibodies described herein may bind to its antigen with affinities as described above with respect to anti-tryptase antibodies.

In one aspect, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)—labeled antigen in the presence of a titration series of unlabeled antigen, then capturing bound antigen with an anti-Fab antibody-coated plate (see, e.g., Chen et al. J. Mol. Biol. 293:865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]—antigen are mixed with serial dilutions of a Fab of interest (e.g., consistent with assessment of the anti-VEGF antibody, Fab-12, in Presta et al. Cancer Res. 57:4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN®-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another aspect K_{D} is measured using a BIACORE® surface plasmon resonance assay. For example, an assay using a BIACORE®-2000 or a BIACORE®-3000 (BlAcore, Inc., Piscataway, N.J.) is performed at 25° C. with immobilized antigen CM5 chips at ˜10 response units (RU). In one aspect, carboxymethylated dextran biosensor chips (CM5, BIACORE, Inc.) are activated with N-ethyl-N′- (3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in phosphate buffered saline (PBS) with 0.05% polysorbate 20 (TWEEN®-20) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (_{KD}) is calculated as the ratio k_{off}/kₒₙ. See, for example, Chen et al. (J. Mol. Biol. 293:865-881, 1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some aspects, K_{D} is measured using a BIACORE® SPR assay. In some aspects, the SPR assay can use a BlAcore®T200 or an equivalent device. In some aspects, BlAcore® Series S CM5 sensor chips (or equivalent sensor chips) are immobilized with monoclonal mouse anti-human IgG (Fc) antibody and anti-tryptase antibodies are subsequently captured on the flow cell. Serial 3-fold dilutions of the His-tagged human tryptase beta 1 monomer (SEQ ID NO: 128) are injected at a flow rate of 30μl/min. Each sample is analyzed with 3 min association and 10 min dissociation. The assay is performed at 25° C. After each injection, the chip is regenerated using 3 M MgCl_{2.} Binding response is corrected by subtracting the response units (RU) from a flow cell capturing an irrelevant IgG at similar density. A 1:1 Languir model of simultaneous fitting of kₒₙ and k_{off} is used for kinetics analysis.

2. Antibody Fragments

In certain aspects, an antibody provided herein (e.g., an anti-tryptase antibody) is an antibody fragment. Antibody fragments include, but are not limited to, Fab, Fab′, Fab′-SH, F(ab′)_{2,} Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134, 2003; and Hollinger et al. Proc. Natl. Acad. Sci. USA 90: 6444-6448, 1993. Triabodies and tetrabodies are also described in Hudson et al. Nat. Med. 9:129-134, 2003.

Single-domain antibodies are antibody fragments comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain aspects, an antibody provided herein (e.g., an anti-tryptase antibody) is a chimeric antibody. Certain chimeric antibodies are described, e.g., in U.S. Pat. No. 4,816,567; and Morrison et al. Proc. Natl. Acad. Sci. USA, 81:6851-6855, 1984). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:1619-1633, 2008, and are further described, e.g., in Riechmann et al. Nature 332:323-329, 1988; Queen et al. Proc. Natl. Acad. ScL USA 86:10029-10033, 1989; US Pat. Nos. 5, 821,337, 7,527,791, 6,982,321, and 7,087,409; Kashmiri et al. Methods 36:25-34, 2005 (describing specificity determining region (SDR) grafting); Padlan, Mol. Immunol 28:489-498, 1991 (describing “resurfacing”); Dall′Acqua et al. Methods 36:43-60, 2005 (describing “FR shuffling”); and Osbourn et al. Methods 36:61-68, 2005 and Klimka et al. Br. J. Cancer, 83:252-260, 2000 (describing the “guided selection” approach to FR shuffling).

Human framework regions that may be used for humanization include but are not limited to: framework regions selected using the “best-fit” method (see, e.g., Sims et al. J. Immunol 151:2296, 1993); framework regions derived from the consensus sequence of human antibodies of a particular subgroup of light or heavy chain variable regions (see, e.g., Carter et al. Proc. Natl. Acad. ScL USA, 89:4285, 1992; and Presta et al. J. Immunol, 151:2623, 1993); human mature (somatically mutated) framework regions or human germline framework regions (see, e.g., Almagro et al. Front. Biosci. 13:1619-1633, 2008); and framework regions derived from screening FR libraries (see, e.g., Baca et al. J. BioL Chem. 272:10678-10684, 1997 and Rosok et al. J. BioL Chem. 271:22611-22618, 1996).

4. Human Antibodies

In certain aspects, an antibody provided herein (e.g., an anti-tryptase antibody) is a human antibody. Human antibodies can be produced using various techniques known in the art. Human antibodies are described generally in van Dijk et al. Curr. Opin. PharmacoL 5:368-74, 2001 and Lonberg, Curr. Opin. Immunol 20:450-459, 2008.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125, 2005. See also, for example, U.S. Patent Nos. 6,075,181 and 6,150,584 describing XENOMOUSE^{TM} technology; U.S. Patent No. 5,770,429 describing HUMAB® technology; U.S. Patent No. 7,041,870 describing K-M MOUSE® technology, and U.S. patent application publication Ser. No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001, 1984; Brodeur et al. Monoclonal Antibody Production Techniques and Applications, pp. 51-63 (Marcel Dekker, Inc., New York, 1987); and Boerner et al. J. Immunol. 147: 86, 1991). Human antibodies generated via human B-cell hybridoma technology are also described in Li et al. Proc. Natl. Acad. Sci. USA, 103:3557-3562, 2006. Additional methods include those described, for example, in U.S. Patent No. 7,189,826 (describing production of monoclonal human IgM antibodies from hybridoma cell lines) and Ni, Xiandai Mianyixue, 26(4):265-268, 2006 (describing human-human hybridomas). Human hybridoma technology (Trioma technology) is also described in Vollmers et al. Histology and Histopathology 20(3):927-937, 2005 and Vollmers et al. Methods and Findings in Experimental and Clinical Pharmacology 27(3):185-91, 2005.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies (e.g., an anti-tryptase antibody) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O′Brien et al., ed., Human Press, Totowa, N.J., 2001) and further described, e.g., in the McCafferty et al. Nature 348:552-554, 1990; Clackson et al. Nature 352: 624-628, 1991; Marks et al. J. Mol. Biol. 222: 581-597, 1992; Marks et al. in Methods in Molecular Biology 248:161-175 (Lo, ed., Human Press, Totowa, N.J., 2003); Sidhu et al. J. Mol. Biol. 338(2): 299-310, 2004; Lee et al. J. Mol. Biol. 340(5): 1073-1093, 2004; Fellouse, Proc. Natl. Acad. Sci. USA 101(34):12467-12472, 2004; and Lee et al. J. Immunol. Methods 284(1-2): 119-132, 2004.

In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol., 12: 433-455, 1994. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. EMBO J. 12: 725-734, 1993. Finally, naive libraries can also be made synthetically by cloning unrearranged V-gene segments from stem cells, and using PCR primers containing random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and U.S. patent publication Ser. Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain aspects, an antibody provided herein (e.g., an anti-tryptase antibody) is a multispecific antibody, for example, a bispecific antibody. Multispecific antibodies are monoclonal antibodies that have binding specificities for at least two different sites. For example, with respect to anti-tryptase antibodies, in certain aspects, bispecific antibodies may bind to two different epitopes of tryptase. In certain aspects, one of the binding specificities is for tryptase and the other is for any other antigen (e.g., a second biological molecule). In some aspects, bispecific antibodies may bind to two different epitopes of tryptase. In other aspects, one of the binding specificities is for tryptase (e.g., human tryptase, e.g., human tryptase beta) and the other is for any other antigen (e.g., a second biological molecule, e.g., IL-13, IL-4, IL-5, IL-17, IL-33, IgE, M1 prime, CRTH2, or TRPA). Accordingly, the bispecific antibody may have binding specificity for tryptase and IL-13; tryptase and IgE; tryptase and IL-4; tryptase and IL-5; tryptase and IL-17, or tryptase and IL-33. In particular, the bispecific antibody may have binding specificity for tryptase and IL-13 or tryptase and IL-33. In other particular aspects, the bispecific antibody may have binding specificity for tryptase and IgE. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein et al. Nature 305: 537, 1983; WO 93/08829; and Traunecker et al. EMBO J. 10: 3655, 1991), and “knob- in-hole” engineering (see, e.g., U.S. Patent No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al. Science, 229: 81, 1985); using leucine zippers to produce bispecific antibodies (see, e.g., Kostelny et al. J. Immunol., 148(5):1547-1553, 1992); using “diabody” technology for making bispecific antibody fragments (see, e.g., Hollinger et al. Proc. Natl. Acad. Sci. USA 90:6444- 6448, 1993); and using single-chain Fv (scFv) dimers (see, e.g., Gruber et al. J. Immunol. 152:5368, 1994); and preparing trispecific antibodies as described, e.g., in Tutt et al. J. Immunol. 147: 60, 1991.

Engineered antibodies with three or more functional antigen binding sites, including “Octopus antibodies,” are also included herein (see, e.g., US 2006/0025576A1).

The antibody or fragment herein also includes a “Dual Acting Fab” or “DAF” comprising an antigen binding site that binds to tryptase as well as another, different antigen (see, US 2008/0069820, for example).

### Knobs-into-Holes

The use of knobs-into-holes as a method of producing multispecific antibodies is described, e.g., in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin. 26:1-9. A brief nonlimiting discussion is provided below. A “protuberance” refers to at least one amino acid side chain which projects from the interface of a first polypeptide and is therefore positionable in a compensatory cavity in the adjacent interface (i.e., the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some aspects, a nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US 2011/0287009 or Table 1 of U.S. Pat. No. 7,642,228.

In some aspects, import residues for the formation of a protuberance are naturally occurring amino acid residues selected from arginine (R), phenylalanine (F), tyrosine (Y) and tryptophan (W). In some aspects, an import residue is tryptophan or tyrosine. In some aspects, the original residue for the formation of the protuberance has a small side chain volume, such as alanine, asparagine, aspartic acid, glycine, serine, threonine, or valine. See, for example, U.S. Pat. No. 7,642,228.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g., by altering nucleic acid encoding the interface). In some aspects, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some aspects, import residues for the formation of a cavity are naturally occurring amino acid residues selected from alanine (A), serine (S), threonine (T), and valine (V). In some aspects, an import residue is serine, alanine, or threonine. In some aspects, the original residue for the formation of the cavity has a large side chain volume, such as tyrosine, arginine, phenylalanine, or tryptophan.

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe, and Trp do not typically extend perpendicularly from the axis of the interface and have preferred conformations, the alignment of a protuberance with a corresponding cavity may, in some aspects, rely on modeling the protuberance/cavity pair based upon a three-dimensional structure such as that obtained by X-ray crystallography or nuclear magnetic resonance (NMR). This can be achieved using widely-accepted techniques in the art. In some aspects, a knob mutation in an IgG1 constant region is T366W. In some aspects, a hole mutation in an IgG1 constant region comprises one or more mutations selected from T366S, L368A, and Y407V. In some aspects, a hole mutation in an IgG1 constant region comprises T3665, L368A, and Y407V.

In some aspects, a knob mutation in an IgG4 constant region is T366W. In some aspects, a hole mutation in an IgG4 constant region comprises one or more mutations selected from T366S, L368A, and Y407V. In some aspects, a hole mutation in an IgG4 constant region comprises T3665, L368A, and Y407V.

7. Antibody Variants

In certain aspects, amino acid sequence variants of the antibodies provided herein are contemplated. For example, it may be desirable to improve the binding affinity and/or other biological properties of the antibody, such as inhibitory activity. Amino acid sequence variants of an antibody (e.g., an anti-tryptase antibody) may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a) Substitution, Insertion, and Deletion Variants

In certain aspects, antibody variants having one or more amino acid substitutions are provided. Sites of interest for substitutional mutagenesis include the HVRs (e.g., CDRs) and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196, 2008), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O′Brien et al. ed., Human Press, Totowa, N.J., 2001). In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In certain aspects, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain aspects of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham et al. Science 244:1081-1085, 1989. In this method, a residue or group of target residues (e.g., charged residues such as Arg, Asp, His, Lys, and Glu) are identified and replaced by a neutral or negatively charged amino acid (e.g., Ala or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b) Glycosylation Variants

In certain aspects, an antibody provided herein (e.g., an anti-tryptase antibody) is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32, 1997. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Patent Publication Ser. Nos. 2003/0157108 and 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249, 2004; Yamane-Ohnuki et al. Biotech. Bioeng. 87: 614, 2004. Examples of cell lines capable of producing defucosylated antibodies include Lec13 CHO cells deficient in protein fucosylation (Ripka et al. Arch. Biochem. Biophys. 249:533-545, 1986; US 2003/0157108; and WO 2004/056312 A1, especially at Example 11), and knockout cell lines, such as alpha-1 ,6-fucosyltransferase gene, FUT8, knockout CHO cells (see, e.g., Yamane-Ohnuki et al. Biotech. Bioeng. 87: 614, 2004; Kanda et al. Biotechnol. Bioeng. 94(4):680-688, 2006; and WO 2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c) Fc Region Variants

In certain aspects, one or more amino acid modifications may be introduced into the Fc region of an antibody (e.g., an anti-tryptase antibody) provided herein, thereby generating an Fc region variant. The Fc region variant may comprise a human Fc region sequence (e.g., a human IgG1, IgG2, IgG3, or IgG4 Fc region) comprising an amino acid modification (e.g., a substitution) at one or more amino acid positions.

In certain aspects, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch et al. Annu. Rev. Immunol. 9:457-492, 1991. Non-limiting examples of in vitro assays to assess ADCC activity of a molecule of interest is described in U.S. Pat. No. 5,500,362 (see, e.g., Hellstrom et al. Proc. Natl. Acad. Sci. USA 83:7059-7063, 1986 and Hellstrom et al. Proc. Natl. Acad. Sci. USA 82:1499-1502, 1985; U.S. Pat. No. 5,821,337 (see Bruggemann et al. J. Exp. Med. 166:1351-1361, 1987). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al. Proc. Natl. Acad. Sci. USA 95:652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al. J. Immunol. Methods 202:163, 1996; Cragg et al. Blood 101:1045-1052, 2003; and Cragg et al. Blood 103:2738-2743, 2004). FcRn binding and in vivo clearance/half-life determinations can also be performed using methods known in the art (see, e.g., Petkova et al. Intl. Immunol. 18(12):1759-1769, 2006).

Antibodies with reduced effector function include those with substitution of one or more of Fc region residues 238, 265, 269, 270, 297, 327 and 329 (U.S. Patent No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312; and Shields et al. J. Biol. Chem. 9(2): 6591-6604, 2001).

In certain aspects, an antibody variant comprises an Fc region with one or more amino acid substitutions which improve ADCC, e.g., substitutions at positions 298, 333, and/or 334 of the Fc region (EU numbering of residues).

In some aspects, alterations are made in the Fc region that result in altered (i.e., either improved or diminished) C1q binding and/or Complement Dependent Cytotoxicity (CDC), for example, as described in US Pat. No. 6,194,551, WO 99/51642, and Idusogie et al. J. Immunol. 164: 4178-4184, 2000.

Antibodies with increased half-lives and improved binding to the neonatal Fc receptor (FcRn), which is responsible for the transfer of maternal IgGs to the fetus (Guyer et al. J. Immunol. 117:587, 1976 and Kim et al. J. Immunol. 24:249, 1994), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan et al. Nature 322:738-40, 1988; U.S. Pat. Nos. 5,648,260 and 5,624,821; and WO 94/29351 concerning other examples of Fc region variants.

d) Cysteine Engineered Antibody Variants

In certain aspects, it may be desirable to create cysteine engineered antibodies, for example, “thioMAbs,” in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain aspects, an antibody provided herein may be further modified to contain additional nonproteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1 ,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, and the like.

In another aspect, conjugates of an antibody and nonproteinaceous moiety that may be selectively heated by exposure to radiation are provided. In one aspect, the nonproteinaceous moiety is a carbon nanotube (Kam et al. Proc. Natl. Acad. Sci USA 102: 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Phamaceutical Formulations

Therapeutic formulations including therapeutic agents used in accordance with the present disclosure (e.g., anti-tryptase antibodies, including any of the anti-tryptase antibodies described herein) are prepared for storage by mixing the therapeutic agent(s) having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol. 119, Marcel Dekker, 2002.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENTM, PLURONICSTM, or polyethylene glycol (PEG).

The formulation herein may also contain more than one active compound, preferably those with complementary activities that do not adversely affect each other. The type and effective amounts of such medicaments depend, for example, on the amount and type of the therapeutic agent(s) present in the formulation, and clinical parameters of the subjects.

The active ingredients may also be entrapped in microcapsules prepared, for example, by coacervation techniques or by interfacial polymerization, for example, hydroxymethylcellulose or gelatin- microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

### V. Articles of Manufacture and Kits

In another aspect, an article of manufacture or kit containing materials useful for the methods and uses described herein is provided. The article of manufacture may include any of the compositions (e.g., anti-tryptase antibodies or compositions thereof (e.g., pharmaceutical compositions)) provided herein. The articles of manufacture and kits may include a container and a label or package insert on or associated with the container. Suitable containers include, for example, bottles, vials, syringes, IV solution bags, and the like. The containers may be formed from a variety of materials such as glass or plastic. The container can hold a composition which is by itself or combined with another composition effective for treating, preventing and/or diagnosing the disorder (e.g., asthma) and may have a sterile access port (for example the container may be an intravenous solution bag or a vial having a stopper pierceable by a hypodermic injection needle). In some aspects, at least one active agent in the composition is an anti-tryptase antibody. The label or package insert indicates that the composition is used for treating the condition of choice. The articles of manufacture or kits can include any of the compositions (e.g., pharmaceutical compositions) described herein. The article of manufacture or kit may include a pump (e.g., a patch pump), e.g., for subcutaneous administration of an anti-tryptase antibody or an antigen-binding fragment thereof. Any suitable pump described herein or known in the art may be included.

For example, provided herein is a kit including any of the anti-tryptase antibodies described herein (e.g., an anti-tryptase beta antibody) and instructions to administer the anti-tryptase antibody to a patient having asthma (e.g., severe asthma (e.g., severe asthma that remains uncontrolled despite standard-of-care therapy) in accordance with any of the methods described herein.

For example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1 D1) of the anti-tryptase antibody selected from 300 mg, 450 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump).

For example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 300 mg. In some aspects, the C1D1 is administered IV. In other aspects, the C1D1 is administered SC (e.g., by a pump (e.g., by a patch pump).

In another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 450 mg. In some aspects, the C1D1 is administered IV. In other aspects, the C1D1 is administered SC (e.g., by a pump (e.g., by a patch pump).

In yet another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 750 mg. In some aspects, the C1D1 is administered IV. In other aspects, the C1D1 is administered SC (e.g., by a pump (e.g., by a patch pump).

In a further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 900 mg. In some aspects, the C1D1 is administered IV. In other aspects, the C1D1 is administered SC (e.g., by a pump (e.g., by a patch pump).

In yet a further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 1350 mg. In some aspects, the C1D1 is administered IV. In other aspects, the C1D1 is administered SC (e.g., by a pump (e.g., by a patch pump).

In a still further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 1800 mg. In some aspects, the C1D1 is administered IV. In other aspects, the C1D1 is administered SC (e.g., by a pump (e.g., by a patch pump).

In another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ) of the anti-tryptase antibody of 3600 mg. In some aspects, the C1D1 is administered IV. In other aspects, the C1D1 is administered SC (e.g., by a pump (e.g., by a patch pump).

In any of the aspects disclosed herein, the dosing cycle may further include one or more additional doses of the anti-tryptase antibody. The dosing cycle may include any suitable number of additional doses (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more additional doses) of the anti-tryptase antibody. For example, in some aspects, the dosing cycle may include a second dose (C1D2). In another example, in some aspects, the dosing cycle may include a C1D2 and a third dose (C1D3). The one or more additional doses may be equal to or unequal to the C1D1. For example, in some aspects, the dosing cycle includes a second dose (C1D2) and a third dose (C1D3) of the anti-tryptase antibody, wherein the C1D2 and the C1D3 are each equal to the C1D1. The one or more additional doses may be administered using any suitable administration route. For example, the one or more additional doses may be administered IV or SC (e.g., by a pump (e.g., by a patch pump).

For example, in one aspect, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are selected from 300 mg, 450 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

For example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump).

In a further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump).

In yet a further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump).

In a still further example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump).

In another example, provided herein is a kit including any of the anti-tryptase antibodies described herein (e.g., an anti-tryptase beta antibody) and instructions to administer the anti-tryptase antibody to a patient having asthma in a dosing regimen including a dosing cycle, wherein the dosing cycle includes a first dose (C1D1 ), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump).

The doses of each dosing cycle may be administered to the subject at any suitable time interval. For example, in some aspects, the doses of the dosing cycle are administered to the subject every four weeks (q4w).

For example, in one aspect, provided herein is a kit including any of the anti-tryptase antibodies described herein (e.g., an anti-tryptase beta antibody) and instructions to administer the anti-tryptase antibody to a patient having asthma at a dose of 300 mg IV, 450 mg IV, 750 mg SC (e.g., by a pump (e.g., by a patch pump), 900 mg IV, 1350 mg IV, 1800 mg IV, or 3600 mg IV every four weeks (q4w).

For example, provided herein is a kit including any of the anti-tryptase antibodies described herein (e.g., an anti-tryptase beta antibody) and instructions to administer the anti-tryptase antibody to a patient having asthma at a dose of 300 mg IV every four weeks (q4w).

In another example, provided herein is a kit including any of the anti-tryptase antibodies described herein (e.g., an anti-tryptase beta antibody) and instructions to administer the anti-tryptase antibody to a patient having asthma at a dose of 450 mg IV every four weeks (q4w).

In yet another example, provided herein is a kit including any of the anti-tryptase antibodies described herein (e.g., an anti-tryptase beta antibody) and instructions to administer the anti-tryptase antibody to a patient having asthma at a dose of 750 mg SC (e.g., by a pump (e.g., by a patch pump) every four weeks (q4w).

In a further example, provided herein is a kit including any of the anti-tryptase antibodies described herein (e.g., an anti-tryptase beta antibody) and instructions to administer the anti-tryptase antibody to a patient having asthma at a dose of 900 mg IV every four weeks (q4w).

In yet a further example, provided herein is a kit including any of the anti-tryptase antibodies described herein (e.g., an anti-tryptase beta antibody) and instructions to administer the anti-tryptase antibody to a patient having asthma at a dose of 1350 mg IV every four weeks (q4w).

In a still further example, provided herein is a kit including any of the anti-tryptase antibodies described herein (e.g., an anti-tryptase beta antibody) and instructions to administer the anti-tryptase antibody to a patient having asthma at a dose of 1800 mg IV every four weeks (q4w).

In another example, provided herein is a kit including any of the anti-tryptase antibodies described herein (e.g., an anti-tryptase beta antibody) and instructions to administer the anti-tryptase antibody to a patient having asthma at a dose of 3600 mg IV every four weeks (q4w).

Each dosing cycle may have any suitable length. For example, in some aspects, each dosing cycle may have a length of about 57 days.

The doses of each dosing cycle may be administered on any suitable day(s) of the dosing cycle. For example, in some aspects, the C1D1 is administered on Day 1 of the dosing cycle, the C1D2 is administered on Day 29 of the dosing cycle, and the C1D3 is administered on Day 57 of the dosing cycle.

The dosing regimens described herein may include any suitable number of dosing cycles. For example, in some aspects, the dosing regimen includes or consists of one dosing cycle. In other aspects, the dosing regimen may include more than one dosing cycle (e.g., 2, 3, 4, 5, 6, 7, 8, 9, 10, 11, 12, 13, 14, 15, 16, 17, 18, 19, 20, or more dosing cycles).

The articles of manufacture and kits may be used for treating any suitable type of asthma. For example, in some aspects, the asthma is moderate asthma. In some aspects, the moderate asthma is uncontrolled despite standard-of-care therapy. In some aspects, the asthma is severe asthma. In some aspects, the severe asthma is uncontrolled despite standard-of-care therapy. In other aspects, the asthma is allergic asthma. In other aspects, the asthma is atopic asthma.

In some aspects, the patient is receiving daily inhaled corticosteroid therapy and at least one of the following controller medications: a long-acting β-agonist (LABA), a leukotriene modulator, a long-acting muscarinic antagonist (LAMA), or a long-acting theophylline preparation.

In some aspects, the leukotriene modulator is a leukotriene modifier (LTM) or leukotriene receptor antagonist (LTRA).

Any suitable anti-tryptase antibody (e.g., anti-tryptase beta antibody) may be used in any of the articles of manufacture and kits described herein. For example, any of the anti-tryptase antibodies described in Section IV, Subsection A above can be used. In some aspects, the anti-tryptase antibody may be any anti-tryptase antibody described in International Patent Application Publication No. WO 2018/148585, which is incorporated herein by reference in its entirety.

For example, any of the articles of manufacture or kits may include an anti-tryptase antibody that includes one, two, three, four, five, or all six of the following complementarity determining regions (CDRs): (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In any of the aspects provided herein, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7; (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8; or (c) a VH domain as in (a) and a VL domain as in (b).

For example, in some aspects, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7. In some aspects, the VH domain includes the amino acid sequence of SEQ ID NO: 7.

In another example, in some aspects, the antibody may include (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8. In some aspects, the VL domain includes the amino acid sequence of SEQ ID NO: 8.

In any of the aspects described herein, the VH domain may include the amino acid sequence of SEQ ID NO: 7 and the VL domain includes the amino acid sequence of SEQ ID NO: 8.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 9 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 9 and (b) a light chain including the amino acid sequence of SEQ ID NO: 10.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 11 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 11 and (b) a light chain including the amino acid sequence of SEQ ID NO: 10.

Any of the articles of manufacture or kits disclosed herein may include one or more additional therapeutic agents. The one or more additional therapeutic agents may be standard of care for asthma. Any suitable standard of care for asthma may be included, e.g., inhaled corticosteroids, long-acting beta agonists, and other controller medications. A person of skill in the art will be able to select a suitable standard of care as appropriate.

### EXAMPLES

The following examples are provided to illustrate, but not to limit the presently claimed invention.

### Example 1: A Phase I, Single-Center, Randomized, Observer-Blinded, Placebo-Controlled Study to Evaluate the Safety, Tolerability, Pharmacokinetics, and Explore Pharmacodynamic Effects and Immunogenicity of Single- and Multiple-Ascending Doses of the Anti-Tryptase Antibody MTPS9579A in Healthy Adult Subjects

1. Study Design

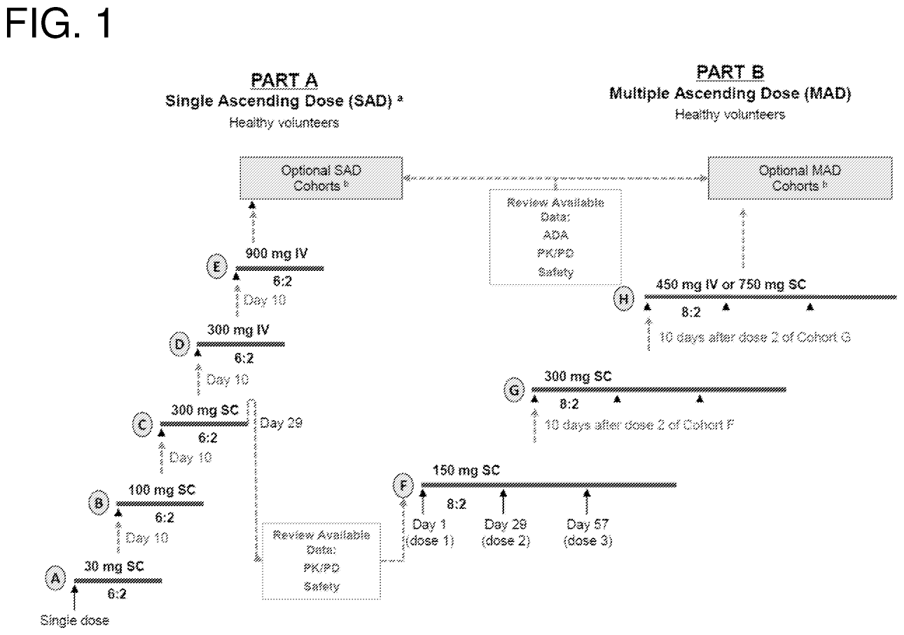

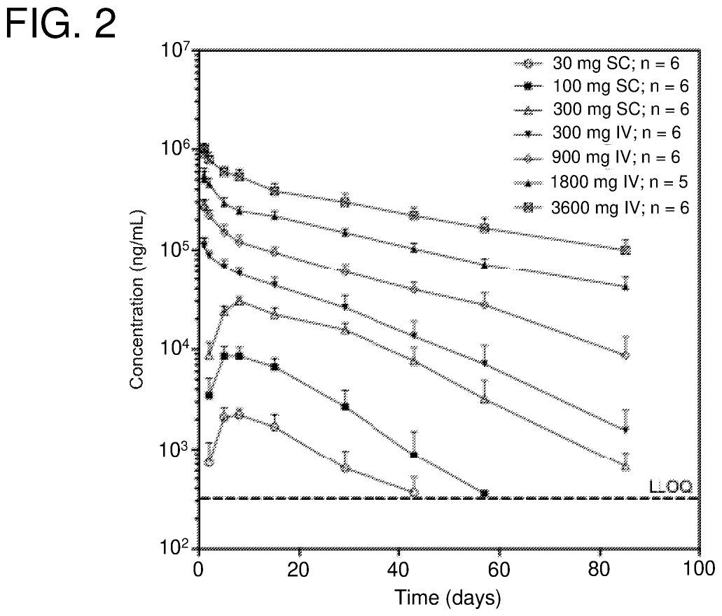

GA40396 is a Phase I, single-center, randomized, observer-blinded, placebo-controlled study to evaluate the safety, tolerability, pharmacokinetics and explore PD effects and immunogenicity of single-ascending doses (SADs; Part A) and multiple-ascending doses (MADs; Part B) of MTPS9579A in healthy male and female adult subjects. Approximately 114 subjects were enrolled in the study with approximately up to 88 subjects total receiving MTPS9579A. FIG. 1 presents an overview of the study design.

A. Part A: Single Ascending Dose (SAD)

Part A comprised ascending, single-dose, sequential cohorts. In total, approximately 40 subjects were studied in an initial 5 cohorts: Cohorts A-E. Each cohort consisted of 8 subjects (6 active:2 placebo). For safety precaution, all SAD cohorts used sentinel dosing. The first 2 subjects of each cohort were randomly assigned to receive either MTPS9579A or placebo (1 subject each). The remainder of the subjects in the respective cohort were allowed to be dosed 24 hours after dosing of the sentinel pair, provided there were no clinically significant safety concerns.

Approximately 30 subjects in total received active treatment with MTPS9579A in Cohorts A-E. However, if the cumulative review of the available PK/PD and safety data of SAD Cohorts A-E revealed that further characterization of the PK, PD, or safety profile of MTPS9579A was needed, optional cohorts were allowed to be added. The dose for each optional cohort was not greater than 2-fold the previously evaluated adequately tolerated dose level and the dose administered did not exceed the pre-specified maximum dose of 3600 mg IV. The proposal to explore additional higher doses was supported by the preliminary safety and tolerability to date. The maximum dose has been increased to 3600 mg (IV). At this dose level, safety margins for MTPS9579A in a 60 kg subject are 2.3-fold on the basis of maximum concentration (Cₘₐₓ), 1.2-fold on the basis of exposure (AUCₛₛ) or 1.7-fold on the basis of dose based on the no-observed-adverse-effects-level (NOAEL) of 100 mg/kg IV, the highest dose tested, in the Good Laboratory Practices (GLP) 3-month repeat dose cynomolgus monkey study.

Potential subjects were screened to assess their eligibility to enter the study within 35 days prior to enrollment. Eligible subjects were enrolled in the study and randomly assigned (6:2 for all cohorts) to receive a single SC or IV dose of MTPS9579A, or matching placebo (on Day 1), in a blinded fashion (see Table 1). Subjects screening to enroll in optional cohorts that explored doses above 900 mg IV were required to have a minimum body weight of 60 kg at screening.

TABLE 1
Part A: Single Ascending-Dose Plan for Cohorts A-E

No. of Subjects
(approximate)

| Cohort | Dose | Route | Active | Placebo |
|---|---|---|---|---|
| A | 30 mg | SC | 6 | 2 |
| B | 100 mg | SC | 6 | 2 |
| C | 300 mg | SC | 6 | 2 |
| D | 300 mg | IV | 6 | 2 |
| E | Up to 900 mg | IV | 6 | 2 |
| Optional | Up to 1800 mg IV | IV | 6 | 2 |
| Cohort 1 | | | | |
| Optional | Up to 3600 mg IV | IV | 6 | 2 |
| Cohort 2 | | | | |

Subjects were required to check in to the clinic on Day -1 and were confined at the clinical site until Day 2. Thereafter, subjects returned to the clinical research unit for required follow-up assessment on subsequent visits till the end of the study (Day 85 (±4 days)). Subjects who discontinued the study prematurely were asked to return to the clinic for an early termination visit 30 (±3) days after the study drug administration, and those who discontinued the study between 30 days after study drug dose and last scheduled visit were asked to return to the clinic for an early termination visit as soon as possible.

A cumulative review of all available pharmacokinetic (PK) and safety data through Day 29 from Cohorts A, B, and C was conducted prior to initiation of multiple dosing in Part B. After Cohort E, available PK/PD, safety, and anti-drug antibody (ADA) data was reviewed to determine whether the optional SAD cohorts should be dosed and what dose(s) should be explored.

B. Part B: Multiple Ascending Dose (MAD)

Part B comprised ascending, multiple-dose, sequential cohorts. In total, approximately 30 subjects were studied in 3 initial cohorts: Cohorts F-H. Each cohort consisted of 10 subjects (8 active:2 placebo). Approximately 24 subjects received active treatment with MTPS9579A in Cohorts F-H. However, if the cumulative review of the available PK, PD, and safety data of MAD Cohorts F-H or other previously dosed cohorts revealed that further characterization of the PK, PD, or safety profile of MTPS9579A was needed, optional Part B cohorts were allowed to be added.

Dosing in the optional Part B cohorts was administered either intravenously or subcutaneously. Dosing of optional Part B cohorts was allowed to commence only after completion of all dose escalation cohorts of Part A and only provided that the IV equivalent dose had been determined to be adequately tolerated in Part A with subjects followed for at least 10 days post-dose.

The dose administered for optional cohorts in Part B was not allowed to exceed those listed in Table 2. Optional cohorts evaluating doses that exceeded 900 mg IV (or SC dose equivalents) were only allowed to be administered to subjects with a body weight of at least 60 kg at screening.

Sentinel dosing was not implemented in Part B given that new adverse events were not expected to be observed with the first dose of a multiple-dose cohort, and staggered dosing would not provide useful safety information. The exposures (along with safety and tolerability) associated with the nominal dose of each MAD cohort have already been investigated in Part A.

Potential subjects were screened to assess their eligibility to enter the study within 35 days prior to enrollment. Eligible subjects were enrolled in the study and randomly assigned (8:2 for all cohorts) to receive doses of MTPS9579A or matching placebo every 4 weeks (04W) for a total of 3 doses (on Days 1, 29, and 57) in a blinded fashion (see Table 2). Lower doses were allowed to be selected for each cohort based on available PK data from Part A at the time of transition to Part B. For example, if higher than anticipated exposures were observed in Part A, then lower doses were allowed to be selected for Part B.

TABLE 2
Part B: Multiple Ascending-Dose Plan for Cohorts F-H

| | | | No. of Subjects | |
|---|---|---|---|---|
| | | | (approximate) | |
| Cohort | Dose | Route | Active | Placebo |
| F | Up to 150 mg | SC | 8 | 2 |
| G | Up to 300 mg | SC | 8 | 2 |
| H | Up to 450 mg IV or | SC or IV | 8 | 2 |
| | 750 mg SC | | | |
| Optional | Up to 1350 mg IV or | SC or IV | 8 | 2 |
| Cohort 1 | equivalent SC dose | | | |
| Optional | Up to 3600 mg IV or | SC or IV | 8 | 2 |
| Cohort 2 | equivalent SC dose | | | |

Subjects were required to check into the clinical research unit 1 day prior to every dosing and were confined at the clinical site until 1 day after dosing. Between the confinement periods and thereafter until the end of the study (Day 141 (±4 days)), subjects returned to the clinical research unit for required follow-up assessment. Subjects who discontinued the study prematurely were asked to return to the clinic for an early termination visit 30 (±3) days after study drug administration, and those who discontinued the study between 30 days after study drug dose and last scheduled visit were asked to return to the clinic for an early termination visit as soon as possible. After the second dose administration for the third MAD cohort, available PK/PD, safety, and ADA data from Parts A and B were reviewed to determine whether optional MAD cohorts should be dosed and what dose(s) should be explored.

C. Dose-Escalation Stage

Approximately 40 subjects were enrolled in Part A (SAD) Cohorts A-E and approximately 30 subjects in Part B (MAD) Cohorts F-H. Additional optional cohorts were evaluated in Part A and Part B. Cohorts were treated at escalating doses in accordance with the dose-escalation rules described below. All SAD cohorts in Part A used sentinel dosing. The first two subjects of each cohort were randomly assigned to receive either MTPS9579A or placebo (one subject each). The remainder of the subjects in the respective cohort were allowed to be dosed 24 hours after dosing of the sentinel pair, provided there were no clinically significant safety concerns.

Prior to each dose escalation, all subjects were monitored for safety for a minimum of 10 days following dosing in the SAD and a minimum of 10 days following the second dose in the MAD. This was true for both planned cohorts (A-H) and optional cohorts. The decision to escalate to the next dose was determined after reviewing all available clinical and safety data, including adverse events, vital signs, and clinical laboratory test results (and PK and exploratory PD data, if applicable).

If a patient was randomized but discontinued early for reasons unrelated to treatment (e.g., lost to follow-up), an additional patient was allowed to be assigned to the associated treatment cohort. Additional cohorts of up to eight more subjects in Part A or 10 subjects in Part B were allowed to be added during dose-escalation safety review to allow adequate exposure to active drug for key safety and/or PK assessments.

D. Dose-Escalation Rules

For Part A, the starting dose of MTPS9579A was 30 mg SC. The dose was increased by up to 3.33-fold of the preceding dose level for each successive cohort, until a dose of 300 mg SC was reached or a safety threshold was observed. Once a dose of 300 mg SC was reached, the route of administration for Part A was changed to IV and a dose of 300 mg IV was administered. The IV dose was increased by up to 3-fold (up to 900 mg IV) of the preceding dose level if no safety event occurred. For Part A, optional cohorts were allowed be added to evaluate IV doses up to 3600 mg. Optional cohorts exploring doses above 900 mg IV did not exceed 2-fold of previously administered, adequately well tolerated (IV) doses. If the PK, PD, and safety objectives were not yet met, additional SC cohorts were also allowed to be added to Part A. SC doses had projected exposures that did not exceed those associated with the highest well-tolerated dose administered IV. Intra-subject dose escalation was not allowed.

After Cohort C, available cumulative PK/PD and safety data from Part A was evaluated to determine commencement of Part B. For Part B, the starting dose of MTPS9579A was 150 mg SC Q4W (Cohort F). Cohort G explored doses up to 300 mg SC 04W. Cohort H explored doses up to 450 mg IV Q4W or 750 mg SC Q4W. If a safety threshold was not observed and if the PK and PD objectives had not yet been met, additional optional Part B cohorts were allowed to commence only after completion of all dose escalation cohorts of Part A and were administered either intravenously or subcutaneously provided that: 1) the IV equivalent dose had been determined to be adequately tolerated in Part A with subjects followed for at least 10 days post-dose; and 2) the IV equivalent dose did not exceed the doses indicated in Table 2. Due to its lower bioavailability, the SC-equivalent dose to the IV dose level exceeded the nominal IV dose indicated in Table 2. Intra-subject dose escalation was not allowed.

E. Stopping Rules

Subjects were required to permanently discontinue study treatment if they experienced any of the following:

Any medical condition that the investigator or Sponsor determined may jeopardize the subject's safety if he or she continues to receive study treatment

Serious adverse event (regardless of relatedness to study drug)

Adverse event Grade related to study drug

Investigator or Sponsor determines it is in the best interest of the subject

Hy's Law

Change in QT interval corrected through use of Fridericia's formula (QTcF) >500 ms or >60 ms change from baseline or episode of torsade de pointes

Pregnancy

Suspicion of hypersensitivity or anaphylactic reaction to study drug

Concurrent illness or requirement for prohibited medication

In the event of subject withdrawal due to a study drug-related adverse event, the subject was expected to complete follow-up procedures. Any subject withdrawn or discontinuing the study prematurely because of a study drug-related adverse event or termination of the study was considered to have completed the study and was not replaced.

F. Cohort Stopping Rules

Dosing of all subjects in a cohort were to be halted for any event whose occurrence suggested that there was significant safety risk to other subjects in a cohort, or a clinically significant pattern of toxicity was apparent in multiple subjects (even if no individual subject was discontinued due to the adverse event). In addition, dosing in a cohort was to be stopped if the following occurred:

Individual stopping rules met for 2 subjects in the same cohort suggestive of a pattern (with the exception of pregnancy) indicating that other subjects in the cohort are at risk for similar adverse drug reactions

Serious adverse event related to study drug occurring in a subject who received active MTPS9579A

Severe (Grade 3 or higher) adverse event, deemed related to study drug and occurring in 2 or more subjects who received active MTPS9579A in the same cohort

If any the above stopping rules were met at any time, dosing was to be suspended for all patients in that cohort, while continuation or initiation of cohorts at lower nominal doses was to be considered. If, after further review of the study data, a determination was made that the stopping rules had not been met or that clear alternative cause(s) for the adverse event was identified (and thus the adverse event was determined to be unrelated to study drug), then dosing of the cohort was allowed to resume.

2. Rationale for Study Design

Healthy volunteers, instead of a patient population with asthma, were chosen for this FIH study in order to safely assess the effects of MTPS9579A. Healthy volunteers rather than patients were enrolled in this study for the following reasons:

Healthy volunteers are expected to have the best systemic reserve to respond to any unexpected reactions to MTPS9579A, such as hypersensitivity reactions

Patients are not participating in this study as they may need to discontinue standard-of-care therapy so that the study could achieve its objective of assessing the safety, tolerability, PK profile, immunogenicity, and exploratory biomarker effects of MTPS9579A alone

The proposed starting and maximum doses for MTPS9579A were selected based on the totality of the data including our understanding of tryptase biology in healthy volunteers and asthma patients, MTPS9579A properties, mechanism of action, nonclinical activity and safety, and prior clinical experience targeting tryptase. In summary:

Active tryptase is secreted from mast cells only upon activation during inflammatory or allergic responses. Active tryptase in tetrameric form is generally not present in systemic circulation, and even in tissue, the active tryptase is held inside mast cells. Moreover, tryptase acts as a secreted protease in response to stimulus as opposed to playing an essential role in homeostatic function. Pharmacological inhibition of tryptase is not anticipated to produce any physiological effect in healthy volunteers as the numbers of mast cells are low and are generally not degranulating under normal physiological conditions.

MTPS9579A is an antagonistic IgG4 antibody targeting a soluble protein, with no direct effect on T-cell activation or cytokine production, nor any agonistic activity on the immune system. No MTPS9579A-related adverse events were observed in the nonclinical toxicological evaluation, including effects on T, B, and NK cells.

The published clinical experience with small molecule inhibitors targeting tryptase, APC366 (inhaled) and APC 2059 (oral), demonstrated pharmacological activity with no adverse events related to the inhibition of tryptase.

The proposed starting dose in healthy volunteers is 30 mg administered subcutaneously. This dose is >98-fold lower than the exposure—, Cₘₐₓ—, and dose-based nonclinical safety margins (see Table 3) determined from a cynomolgus monkey (cyno) toxicology study at a no observed adverse effect level (NOAEL) of 100 mg/kg IV. Based on PK/PD modeling, the maximum target inhibition of active tryptase in the lung in healthy volunteers by MTPS9579A was predicted to be 60%-75% at this dose level. However, the starting dose was not anticipated to produce any physiological effect in healthy volunteers, since active tryptase is secreted from mast cells upon activation during inflammatory or allergic responses. Healthy volunteers with a history or allergy or anaphylaxis were excluded from this study. For all the reasons described above, 30 mg SC was an appropriate starting dose for MTPS9579A.

The extent of target inhibition necessary for efficacy could depend on tryptase levels, which can be highly variable between subjects and can be impacted by disease status and severity. The proposed maximum dose in healthy volunteers is 3600 mg administered intravenously. This maximum dose was selected following evaluation of preliminary clinical PK data from Part A of the ongoing study. The available data from Part A indicated that the Cₘₐₓ was lower and the half-life of MTPS9579A was shorter than originally predicted by the nonclinical model. Patients with asthma have higher tryptase levels than healthy volunteers, and MTPS9579A potentially undergoes target-mediated clearance; therefore, without wishing to be bound by theory, doses required to achieve adequate target inhibition may be substantially higher in patients relative to healthy subjects. Based on these data, and without wishing to be bound by theory, doses as high as 3600 mg IV may be needed to saturate the target in the lung.

At this maximum dose of 3600 mg IV, >1.2-fold exposure—, Cₘₐₓ—, and dose-based safety margins were established based on the GLP, repeat-dose, toxicology study at a NOAEL of 100 mg/kg IV for subjects weighing at least 60 kg (see Table 3). No MTPS9579A-related adverse effects were observed in the toxicology study and 100 mg/kg was the highest dose tested in the study. For the FIH SAD part of the study, 5 initial dose cohorts were planned, with 3- to 3.33-fold increases in dose between each cohort up to 900 mg IV (>3-fold safety margin to NOAEL in the monkey GLP toxicology study) to allow for adequate separation of exposure. Doses above 900 mg IV and up to 3600 mg IV were explored in optional SAD cohorts, based on adequate safety and tolerability. Dose steps between increasing dose levels in Part A were not allowed to exceed 2-fold the previous highest tolerated SAD dose, and subjects entering optional cohorts evaluating dose levels >900 mg IV (or SC equivalent) were required to have a minimum body weight of 60 kg, to enable at least a 1.2-fold safety margin (see Table 3).

Each SAD cohort had 2 sentinel subjects (1 receiving active drug). Starting at a dose of 30 mg allowed for dose escalations to occur before reaching the anticipated therapeutic dose range, which is estimated to be 300 mg SC to 900 mg IV for healthy volunteers. The anticipated therapeutic range in disease is broad as a large range of mast cell degranulation is possible, leading to a wider range of active tryptase that must be inhibited in the target organ.

Dose escalations within the SAD portion and within the MAD portion were guided by safety information. Pharmacokinetics as measured by drug levels in the serum are considered to be less relevant for defining active exposure range as tryptase monomers in circulating blood are inactive. After Cohort C of the SAD, transition to the first MAD Cohort was guided by available cumulative PK/PD and safety data. Three initial cohorts were planned for the MAD of the study with 2-3-fold increases in dose between cohorts. Additional optional MAD cohorts were evaluated at or below doses evaluated and determined by the SMC to be adequately tolerated in Part A. The dose equivalents explored did not exceed those indicated in Table 2. Subjects entering an optional MAD cohort evaluating doses levels >900 mg IV (or SC equivalent) were required to have a minimum body weight of 60 kg, to enable at least a 1.2-fold safety margin (see Table 3).

See Table 3 for safety margin estimates.

TABLE 3
Safety Margin Estimates for MTPS9579A
across the Phase I Clinical Doses

| | | | |
|---|---|---|---|
| Phase I Dose | AUC^{a} | Cₘₐₓ^{b} | Dose^{c} |

Starting SAD (30 mg SC)

| | | | |
|---|---|---|---|
| 40 kg subject | 115 | 406 | 133 |
| 70 kg subject | 201 | 711 | 233 |
| 120 kg subject | 345 | 1220 | 400 |

SAD (900 mg IV)

| | | | |
|---|---|---|---|
| 40 kg subject | 3 | 6 | 4 |
| 70 kg subject | 6 | 11 | 8 |
| 120 kg subject | 10 | 18 | 13 |

MAD (750 mg × 3 SC)

| | | | |
|---|---|---|---|
| 40 kg subject | 8 | 10 | 12 |
| 70 kg subject | 13 | 17 | 19 |
| 120 kg subject | 23 | 30 | 37 |

MAD (450 mg × 3 IV)

| | | | |
|---|---|---|---|
| 40 kg subject | 11 | 9 | 21 |
| 70 kg subject | 19 | 16 | 36 |
| 120 kg subject | 32 | 27 | 62 |

Maximum SAD (3600 mg IV)

| | | | |
|---|---|---|---|
| 60 kg subject | 1.2 | 2.3 | 1.7 |

Maximum MAD (3600 mg × 3 IV)

| | | | |
|---|---|---|---|
| 60 kg subject | 2.0 | 1.7 | 3.9 |

AUC = area under the concentration-time curve; Cₘₐₓ = maximum serum concentration observed; TK = toxicokinetic.
Note:
The subject weight range is 40-120 kg for cohorts up to 900 mg IV. Subjects must weigh at least 60 kg for (optional) cohorts evaluating doses above 900 mg IV.
^{a}Exposure-based safety margin; AUC_{cyno} for SAD safety margins calculated using AUCₛₛ (AUC₇₀₋₈₄); AUC_{cyno} for MAD safety margins calculated using AUCₐₗₗ (AUC₀₋₈₇).
^{b}Concentration-based safety margin; C_{max-cyno} following last dose on Study Day 85 (TK Day 84).
^{c}Dose-based safety margin; Dose_{cyno} for SAD safety margin calculated using single-dose of 100 mg/kg, Dose_{cyno} for MAD safety margin calculated using 100 mg/kg × 7 doses (700 mg/kg).

This study was placebo controlled to avoid bias in the collection and evaluation of data during its conduct. Placebo has been chosen as the control treatment to assess whether any observed effects or safety outcomes are treatment-related or simply reflect the study conditions.

Biomarkers were measured in serum and using a technique called nasosorption to observe evidence of the biologic activity of MTPS9579A in subjects, identify biomarkers that are predictive of response to MTPS9579A, define PK/PD relationships, and support selection of a recommended dose regimen. Nasosorption is a non-invasive sampling method that uses a synthetic absorptive matrix to collect nasal mucosal lining fluid from the nose.

3. Materials and Methods

A. Subjects

Approximately 114 healthy, male and female volunteers between the ages of 18 and 55 years were enrolled at one investigative site.

B. Inclusion Criteria

Subjects were required to meet the following criteria for study entry:

Signed Informed Consent Form

Age ≥18 and ≤55 years at time of signing Informed Consent Form

Ability to comply with the study protocol, in the investigator's judgment

Body mass index of ≥18.0 to ≤32.0 kg/m² or, if outside the range, considered not clinically significant by the investigator and approved by the Medical Monitor
For any optional cohort in either Part A or B evaluating doses >900 mg IV (or SC equivalent): Must have a body weight ≥60 kg at screening

In good health, determined by no clinically significant findings from medical history, 12-lead electrocardiogram (ECGO, and vital signs. Vital signs at rest at screening should be within the following ranges:
Oral body temperature ≥35 to ≤37° C.
Systolic blood pressure ≥90 to ≤140 mmHg
Diastolic blood pressure ≥50 to ≤90 mmHg

QuantiFERON® TB Gold negative result at screening

C. Exclusion Criteria

Subjects who met any of the following criteria were excluded from study entry:

Pregnant or breastfeeding, or intending to become pregnant during the study or within 110 days after the last dose of study drug
Women of childbearing potential must have a negative urine pregnancy test at screening and a negative serum pregnancy test on Day -1

Treatment with investigational therapy within 3 months or 5 drug half-lives, whichever is longer, prior to screening

Planned surgical intervention during the study

Positive for hepatitis C virus (HCV) antibody, hepatitis B surface antigen (HBsAg), hepatitis B core antibody (HBcAb) or human immunodeficiency virus (HIV) antibody at screening

History of a positive tuberculin skin test in subjects who are bacille Calmette-Guérin vaccine naïve, history of a positive interferon-gamma release assay, or history of latent or active tuberculosis or exposure to endemic areas within 8 weeks prior to screening

Illicit drug or alcohol abuse within 2 years prior to screening, in the investigator's judgment or positive drugs of abuse test result at screening

Regular alcohol consumption in males >15 units per week and females >10 units per week (1 unit =8 oz beer, 1 oz of 40% spirit or a 4 oz glass of wine)

Current smokers, including tobacco, marijuana, use of electronic cigarettes (i.e., vaping), or nicotine replacement products, and those who have smoked or used these products within the last 12 months or a positive urine cotinine test

Poor peripheral venous access as assessed by the investigator or delegate at screening

Any serious medical condition or abnormality in clinical laboratory tests that, in the investigator's judgment, precludes the subject's safe participation in and completion of the study

Receipt of blood products within 2 months prior to screening

History of malignancy, except for appropriately treated carcinoma in situ of the cervix, non-melanoma skin carcinoma, or Stage I uterine cancer

Donation or loss of blood (excluding the volume of blood that will be drawn during screening procedures) as follows: 50-499 mL of blood within 30 days or >499 mL of blood within 2 months prior to screening

History of clinically significant cardiovascular, renal, hepatic, chronic respiratory or gastrointestinal disease, or psychiatric disorder, as judged by the investigator

Evidence of renal impairment at screening, as indicated by an estimated creatinine clearance (estimated glomerular filtration rate (eGFR)) of <70 mL/min using the Chronic Kidney Disease Epidemiology Collaboration (CKD-EPI) equation

History or presence of an abnormal ECG that is clinically significant in the investigator's opinion, including complete left bundle branch block, second- or third-degree heart block, or evidence of prior myocardial infarction.

QTcF ≥450 ms, if subject is male, or QTcF ≥470 ms, if subject is female, demonstrated by the average of a triplicate of ECGs at screening

Current treatment with medications that are well known to prolong the QT interval

History of anaphylaxis, hypersensitivity, or significant drug allergies

Presence or history of clinically significant allergy requiring treatment, as judged by the investigator
History of hay fever is allowed unless it is active

Clinical diagnosis of asthma requiring treatment in the last 12 months

Upper or lower respiratory tract infection within 4 weeks prior to screening

Received oral antibiotics within 4 weeks prior to screening, or IV/intramuscular (IM) antibiotics within 8 weeks prior to screening

Hospitalization within 4 weeks prior to screening

Subjects who are taking, or have taken, any prescribed or over-the-counter drug or herbal remedies in the 14 days or 5 half-lives, whichever is longer, before screening
Exceptions may apply on a case-by-case basis, if considered not to interfere with the objectives of the study

Received live or attenuated vaccine (including but not limited to FluMist®-brand influenza vaccine; measles, mumps, rubella; varicella zoster/chickenpox; oral polio; etc.) within 30 days prior to screening

Received killed vaccine (including seasonal flu and H1N1 vaccine) within 14 days prior to screening, unless deemed acceptable

Inability to comply with study protocol

D. Method of Treatment Assignment and Blinding

This is a randomized, observer-blinded, placebo-controlled study. Approximately 114 subjects were randomized and treated with study drug (MTPS9579A or placebo). The study subjects and the site staff remained blinded to treatment assignment at all times, with the exception of the study pharmacist(s) who prepared and dispensed the study medication and were aware of the randomization code. The unblinded site pharmacist will maintain a list of treatment assignments to ensure that subjects receive either MTPS9579A or placebo in a 6:2 ratio for cohorts in Part A (SAD) and an 8:2 ratio for cohorts in Part B (MAD).

E. Study Treatment and other Treatments Relevant to the Study Design

The investigational medicinal product (IMP) for this study is MTPS9579A. MTPS9579A was supplied as sterile liquid in 2-cc glass vials. MTPS9579A is provided in single-dose, USP/Ph. Eur. Type I colorless borosilicate vials for injection. The approximate concentration of MTPS9579A in the vials was 150 mg/mL. MTPS9579A placebo was provided in single-dose 2 mL, USP/Ph. Eur. Type I colorless borosilicate vials for injection. Treatment regimens are summarized above (e.g., in Tables 2 and 3) and in FIG. 1.

For SC administration, undiluted study drug (150 mg/mL concentration) was administered by SC injection using syringes. For administration of the 30 mg dose (0.2 mL volume) and the 100 mg dose (0.67 mL volume), a 1.0-mL syringe should be used. For administration of the 300 mg dose (2.0 mL volume or 2x1.0 mL volume) or greater, a larger syringe (up to 5.0 mL) may be used. To ensure adequate precision, syringe sizes of 10 mL or larger were not used. Multiple injections may have been required for higher dose levels. All SC injections were expected to be administered in the abdomen, if possible. Use of an alternate injection site could be considered if needed to ensure SC rather than IM injection. The preferred alternate injection site was the back of the upper arm.

For IV administration, the doses were prepared by diluting study drug with saline. Once diluted, study drug was administered IV at a rate of 1.5 mL per minute. For the 300 mg dose (approximately 100 mL at approximately 3.0 mg/mL concentration), the infusion required approximately 67 minutes, and the dose was expected to be given in its entirety. For the 900 mg dose (approximately 100 mL at approximately 9.0 mg/mL concentration), the infusion required approximately 67 minutes, and the dose was expected to be given in its entirety. For optional cohorts evaluating doses above 900 mg, the concentration was adjusted to deliver a total volume of approximately 100 mL per dose (e.g., the highest possible dose of 3600 mg was approximately 100 mL at approximately 36 mg/mL concentration). For subjects who experienced mild infusion-related signs or symptoms (<Grade 2), the infusion time was allowed to be modified. For subjects with infusion-related signs or symptoms requiring treatment, the infusion was expected to be discontinued. Subjects were expected to not be medicated or premedicated in order to tolerate IV administration of study drug.

F. Study Assessments

Subjects were closely monitored for safety and tolerability throughout the study. Subjects were expected to be assessed for toxicity prior to each dose; dosing occurred only if the clinical assessment and local laboratory test values were acceptable.

Medical history, including clinically significant diseases, surgeries, cancer history (including prior cancer therapies and procedures), reproductive status, smoking history, and use of alcohol and drugs of abuse, were recorded at baseline. In addition, all medications (e.g., prescription drugs, over-the-counter drugs, vaccines, herbal or homeopathic remedies, nutritional supplements) used by the subject within 7 days prior to initiation of study treatment were recorded. At the time of each follow-up physical examination, an interval medical history was expected to be obtained and any changes in medications and allergies were expected to be recorded.

Demographic data included age, sex, and self-reported race/ethnicity.

A complete physical examination, performed at screening and other specified visits, was expected to include an evaluation of the head, eyes, ears, nose, and throat, and the cardiovascular, dermatologic, musculoskeletal, respiratory, gastrointestinal, and neurologic systems. Limited, symptom-directed physical examinations could be performed at specified postbaseline visits and as clinically indicated. Changes from baseline abnormalities were recorded. New or worsened clinically significant abnormalities were recorded as adverse events in the appropriate source.

Vital signs included measurements of respiratory rate, pulse rate, pulse oximetry, and systolic and diastolic blood pressure while the subject was in a seated position (resting for at least 5 minutes), and oral temperature. Vital signs were monitored serially (every 15 min (±3 min)) during study drug administration and for the first hour immediately after dosing. Vital signs were performed within 20 minutes prior to dosing; and every hour (±10 minutes) starting from the end of the dose up to 6 hours post-dose.

Nasosorption is a minimally invasive technique that samples the nasal mucosal lining fluid using the NASOSORPTION™FX-i-device (Hunt Developments, available as CE-marked device). This device has been used in humans. The aseptic was is inserted into a nostril with the absorbent strip held flat against the surface of the inferior turbinate for 60 seconds.

Samples for the following laboratory tests were sent to the study site's local laboratory for analysis:

Hematology: white blood cell (WBC) count, red blood cell (RBC) count, hemoglobin, hematocrit, platelet count, differential count (neutrophils, eosinophils, basophils, monocytes, lymphocytes, other cells)

Chemistry panel (serum or plasma): sodium, potassium, chloride, bicarbonate, glucose, blood urea nitrogen (BUN) or urea, creatinine, creatine phosphokinase, total protein, albumin, phosphorus, calcium, magnesium, total and direct bilirubin, alkaline phosphatase (ALP), alanine aminotransferase (ALT), aspartate aminotransferase (AST), gamma glutamyl transferase, eGFR (Chronic Kidney Disease Epidemiology Collaboration (CKD-EPI))

Coagulation: International Normalized Ratio (INR), activated partial thromboplastin time (aPTT), prothrombin time (PT), fibrinogen, fibrinogen split products (contingent on assay availability)

Viral serology: HIV, HBsAg, total HBcAb, HCV antibody

Tuberculosis (TB) test: QuantiFERON®-TB Gold

Pregnancy test
All women of childbearing potential had a urine pregnancy test at screening. Serum pregnancy tests were performed at specified subsequent visits.

Urinalysis, including dipstick (pH, specific gravity, glucose, protein, ketones, bilirubin, leukocytes, nitrite, blood); and microscopic examination (sediment, RBCs, WBCs, casts, crystals, epithelial cells, bacteria) was assessed

Urine drug screen

Alcohol test
All subjects had a breath test (or breathalyzer) to detect recent alcohol consumption at check-in the day prior to dosing days.

Urine cotinine test

Serum samples for PK analysis
MTPS9579A in samples was quantified using a validated assay.

Serum samples for immunogenicity assessment
Serum samples were collected to assess immunogenicity of MTPS9579A by measuring ADAs. ADA serum samples were collected prior to study drug administration on dosing days, and ADA was detected and characterized using validated assays.

Nasosorption samples for PK analysis

Serum, nasosorption, and blood DNA samples for exploratory research on biomarkers

Exploratory biomarker research can include, but is not limited to, active tryptase, total tryptase, and urea.

Subjects underwent safety monitoring during the study, including assessment of the nature, frequency, and severity of adverse events. In general, the WHO toxicity grading scale was used for assessing adverse event severity. Safety assessments included monitoring and recording adverse events, including serious adverse events and adverse events of special interest, performing protocol-specified safety laboratory assessments, measuring protocol-specified vital signs, and conducting other protocol-specified tests that were deemed critical to the safety evaluation of the study. According to the ICH guideline for Good Clinical Practice, an adverse event is any untoward medical occurrence in a clinical investigation subject administered a pharmaceutical product, regardless of causal attribution. An adverse event can therefore be any of the following:

Any unfavorable and unintended sign (including an abnormal laboratory finding), symptom, or disease temporally associated with the use of a medicinal product, whether or not considered related to the medicinal product

Any new disease or exacerbation of an existing disease (a worsening in the character, frequency, or severity of a known condition)

Recurrence of an intermittent medical condition (e.g., headache) not present at baseline

Any deterioration in a laboratory value or other clinical test (e.g., ECG, X-ray) that was associated with symptoms or leads to a change in study treatment or concomitant treatment or discontinuation from study drug

Adverse events that were related to a protocol-mandated intervention, including those that occured prior to assignment of study treatment (e.g., screening invasive procedures such as biopsies)

Reduction in dosing for adverse events was not permitted. Subjects who experienced certain adverse events considered to be related to study drug were expected to be discontinued from treatment.

G. Biomarker Methods for Clinical Pharmacodynamics Analysis

Human Serum and Naso Total Tryptase Gyros Assay:

The assay for the detection of human total tryptase in serum and nasosorption samples was performed using a Gyros GYROLAB® (xP or Workstation) system. This is a flow-through immunoassay platform, utilizing miniature columns containing streptavidin-coated beads that are sequestered within disposable microfluidic compact discs (CDs) that also contain reagent and sample handling microstructures. First, the biotinylated capture antibody (Clone: E88AS) was immobilized onto beads within each column. Next, the standards, controls and samples, pre-diluted within assay diluent containing anti-tryptase MTPS9795A, were added, and bound to the respective columns. Finally, an ALEXA®-647-labeled detection antibody (Clone: E82AS) was added to the columns. To determine the amount of fluorescence (i.e., amount of captured protein) per structure, each CD was automatically transferred to a laser-induced fluorescence (LIF) detector, which was incorporated into GYROLAB®. Detection at the 5% photo multiplier tube (PMT) setting was used to generate sample analysis data. In this assay, the fluorescence signal is proportional to the amount of total tryptase bound to each column.

The total tryptase concentrations were determined from a standard curve by plotting response (fluorescence) versus concentration using a five-parameter logistic curve-fitting program with the “weighting by response” option selected (1/y² weighting). The calibration curve range of this method was from 800-0.122 ng/ml. However, the reportable range of the assay, ULOQ to LLOQ, was 267-0.366 ng/ml.

Human active tryptase SIMOA® assay procedure:

The SIMOA® active tryptase assay was based on the HomeBrew protocol as described in the QUANTERIX® manual, using the monoclonal antibodies E88AS to capture labelled and dissociated tryptase onto beads. Briefly, capture beads and antibody were prepared by buffer exchange into the

QUANTERIX®-recommended Bead Conjugation Buffer using AMICON® Ultra-0.5 centrifugal filters. Conjugation of the capture antibody to beads was based on 1-ethyl-3-(3-dimethylaminopropyl) carbodiimide (EDAC) chemistry and performed according to QUANTERIX® manual protocol. Beads were further characterized through the bead aggregation protocol on the SIMOA® HD-1 ANALYZERTM (QUANTERIX®) for bead number and aggregation level, as recommended. Bead characterization showed that greater than 95% of the bead mixture generated was monomeric. The detection reagent was the biotinylated-activity-based probe (ABP) molecule (see U.S. Patent Application Publication Ser. No. 2018/0230233) which was used to bind the active site of the enzyme during sample preparation. A two-step protocol, where a sample is co-incubated with capture beads and detection reagent (for 30min) in a single step, followed by the sequential addition and washing of streptavidin-beta galactosidase (SBG 100pM) and the fluorescent substrate RGP (resorufin-(3-D-galactopyranoside) for signal generation was utilized. No detection antibody was required, as biotinylated ABP was used to bind SBG for detection. SIMOA® analysis software was used to quantify all samples, using a 4-PL fit of standards with a 1/Y² weighting.

H. PK Analysis

Serum PK:

A quantitative assay was designed to detect MTPS9579A in human serum from healthy (normal) individuals and patients with asthma. A standard curve was prepared fresh on the day of use in Standard Diluent (phosphate-buffered saline (PBS), pH 7.4+0.5% bovine serum albumin (BSA)+0.25% 3-[(3-cholamidopropyl)dimethylammonio]-1-propanesulfonate (CHAPS), 5 mM ethylenediaminetetraacetic acid (EDTA), 350 mM NaCI, 0.05% TWEEN® 20 +0.05% PROCLINTM 300 +0.5% NHS (Normal Human Serum) +50 μg/mL Murine IgG (MuIgG)). Quality controls (QCs) and unknown samples were diluted to the assay minimum required dilution (MRD) 1/200 with assay diluent containing 50.0 μg/mL MuIgG. A plate was coated with recombinant rabbit monoclonal antibody (mAb IgG) clone 12D10 for 16 to 72 hours then washed and blocked with Blocking Buffer (PBS, pH 7.4 +0.5% BSA +0.05% TWEEN® 20 +0.05% PROCLINTM 300) for 2 to 3 hours. After an additional wash step, the matrix blank, calibrators, and diluted controls and unknown samples were transferred to the pre-coated and blocked plate and incubated at room temperature for 16 to 20 hours. During this incubation, MTPS9579A in samples was bound to the immobilized rabbit mAb IgG clone 12D10, an anti-idiotype antibody that specifically recognizes the CDR region of MTPS9579A. Unbound materials were removed with a wash step. Mouse anti-human IgG4 Fc-horseradish peroxidase (HRP) was then added to the plate for detection and incubated for 1 hour. Unbound material was then removed with a final wash step. Finally, tetramethylbenzidine peroxidase (TMB) substrate is added to the plate to develop color. The substrate development was stopped after approximately 10 to 20 minutes by adding 1 M phosphoric acid. The plate was read on a plate reader at 450 nm for detection absorbance and at 630 nm for reference absorbance.

Nasosorption Drug Level (Naso PK):

A qualitative assay was designed to detect MTPS9579A in human nasosorption eluates, from healthy (normal) individuals and patients with asthma. A standard curve was prepared fresh on the day of use in Standard Diluent. Quality controls (QCs) and unknown samples were diluted to the assay MRD 1/200 with assay diluent containing 50.0 μg/mL MuIgG. A plate was coated with recombinant rabbit monoclonal antibody (mAb IgG) clone 12D10 for 16 to 72 hours then washed and blocked with Blocking Buffer for 2 to 3 hours. After an additional wash step, the matrix blank, calibrators, and diluted controls and unknown samples were transferred to the pre-coated and blocked plate and incubated at room temperature for 16 to 20 hours. During this incubation, MTPS9579A in samples was bound to the immobilized rabbit mAb IgG clone 12D10. Unbound materials were removed with a wash step. Mouse anti-human IgG4 Fc-ALEXA® FLUOR is then added to the plate for detection and incubated for 1 hour. Unbound material was then removed with a wash step and elution buffer was added to all assay wells for 15 minutes prior to being neutralized by addition of Tris base. Finally, the eluted fluorophore was transferred onto glass bottom plates and fluorescence was quantified on an SMCxPROTM immunoassay instrument.

I. ADA Analysis

Serum samples were evaluated for the presence of ADAs using bridging immunoassays. A tiered approach to testing was implemented. First, a screening assay was implemented to detect ADA to MTPS9579A. Samples testing positive in the screening assay were subsequently subjected to a confirmatory assay, by competition with excess MTPS9579A, to demonstrate that ADAs are specific for the therapeutic protein product. All positive ADA samples were titered. The screen and confirmatory assays targeted 5% and 1% untreated-positive rate, respectively. Samples that were confirmed positive were then diluted further to obtain a value in titer units that is defined as log₁₀(dilution factor).

The ADA assay is a qualitative assay designed to detect antibodies to MTPS9579A in human serum, and uses two conjugated reagents to capture antibodies directed against MTPS9579A: biotin-conjugated MTPS9579A and digoxigenin (DIG)-conjugated MTPS9579A. These two conjugated reagents were co-incubated overnight at room temperature with the diluted controls and samples. Due to the high post-dose levels of tryptase, which is a multimeric protein and can interfere in the ADA assay, by bridging conjugated MTPS9579A, a blocking antibody was added to the assay diluent. The blocking antibody reagent was shown to bind to the same epitope on the tryptase molecule, but due to different CDR's, did not interfere with binding to ADA in the samples. Following a wash step, the control (or sample)/biotin/digoxin solution is transferred to a streptavidin-coated high-bind plate and incubated at room temperature. Following an additional wash step, a solution of HRP conjugated to mouse anti-digoxin antibody was added for detection to the appropriate wells of the streptavidin-coated high-bind plate and incubated at room temperature. After a final wash step, a peroxidase substrate (tetramethylbenzidine) was added to the plate for color development and the reaction was stopped by adding 1 M phosphoric acid. The plate was read on a plate reader at 450 nm (detection) with a 630 nm reference filter.

This assay utilized a minimum 10.0 μI_{—} human serum aliquot for screening (Tier 1) and titer (Tier 3) analyses and a 20.0 μI_{—} human serum aliquot for confirmatory (Tier 2) analysis. Samples were stored frozen in polypropylene tubes at approximately −80 ° C. prior to analysis. The MRD for this assay was determined to be 1/20 in Control/Sample Diluent (CSD).

### Example 2: Results from Study GA40396

As described in Example 1, Study GA40396 is a Phase I, randomized, placebo-controlled, observer-blinded study that is evaluating the safety, tolerability, PK, immunogenicity, and PD of single-and multiple-ascending IV and SC doses of MTPS9579A. This study is focused on the nature, frequency, and severity of serious and non-serious AEs, as well as the effects of study drug treatment on laboratory values, vital sign measurements, ECG parameters, and other safety measures.

As of the safety database cutoff date of April 19, 2019, a total of 106 subjects have completed dosing and safety follow-up in both SAD and MAD cohorts in Study GA40396.

The SAD portion of Study GA40396 has completed evaluation of seven cohorts of 8 subjects each (56 subjects total) in a 6:2 MTPS9579A:placebo ratio. All SAD cohorts included sentinel dosing (1 active, 1 placebo) 24 hours prior to dosing the complete cohort.

The MAD portion of Study GA40396 evaluated five cohorts of 10 subjects each (50 subjects total) in an 8:2 MTPS9579A:placebo ratio. Subjects in the MAD were administered 3 cumulative doses spaced 04W (dosing conducted on Day 1, Day 29, and Day 57).

A. Clinical Pharmacokinetics

Interim PK data are summarized in Table 4, and PK analyses included samples that were obtained from single-dose cohorts (30 mg SC to 3600 mg IV) and multiple-dose cohorts (150 mg SC to 3600 mg IV). The peak serum concentrations were observed 8 days after SC administration. The Cₘₐₓ values increased dose proportionally between 300 to 3600 mg IV. The mean half-life values in the SAD portion of the study were generally shorter at the lower doses, potentially (and without wishing to be bound by any particular theory) because of target-mediated clearance at lower anti-tryptase serum concentrations. At saturating doses (1800-3600 mg IV), terminal t_{1/2} of the linear range was calculated to be 29-34 days by non-compartmental analysis (NCA) and matched the t_{1/2} projected by compartmental modeling analysis (˜30 days).

TABLE 4
Geometric Mean (CV %) Pharmacokinetic Parameter Estimates of MTPS9579A after SC or IV administration
in Healthy Subjects in SAD (GA40396) (N = 6; All Dose Cohorts Unless Noted)

| | Dose | tₘₐₓ^{a} | | Cₘₐₓ | | AUC₀₋ₜ^{b} | | t_{1/2}^{b} | | AUC_{inf}^{b} | | CL^{b} | | V_{z} | |
|---|---|---|---|---|---|---|---|---|---|---|---|---|---|---|---|
| Route | (mg) | (day) | | (μg/mL) | | (μg · day/mL) | | (d) | | (μg · day/mL) | | (L/day) | | (L) | |
| SC | 30 | 8 | (5, 15) | 2.28 | (18.3) | 41.7 | (15.6)^{e} | 11.2 | (16.8)^{c} | 52.9 | (7.44)^{c} | 0.567 | (7.44)^{c} | 9.16 | (9.3)^{c} |
| | 100 | 8 | (5, 15) | 8.89 | (22.7) | 157 | (19.2) | 8.38 | (19.3) | 185 | (21.1) | 0.539 | (21.1) | 6.52 | (18.7) |
| | 300 | 8 | (5, 8) | 30.3 | (8.67) | 574 | (10.3) | 13.3 | (10.7) | 847 | (15.2) | 0.354 | (15.2) | 6.78 | (12.4) |
| IV | 300 | 1.08 | (1.02-1.08) | 111 | (21.2) | 1360 | (17.7)^{d} | 14.59 | (18.9)^{d} | 1970 | (17.8)^{d} | 0.152 | (17.8)^{d} | 3.21 | (23.1) |
| | 900 | 1.05 | (1.02-1.08) | 268 | (18.7) | 2970 | (16.8) | 22.1 | (17.5) | 4950 | (23.0) | 0.187 | (23.0) | 5.79 | (11.3) |
| | 1800^{d} | 1.02 | (1.02-1.08)^{d} | 540 | (18.7)^{d} | 6490 | (11.3)^{d} | 29.1 | (11.7)^{d} | 12700 | (14.1)^{d} | 0.136 | (14.3)^{d} | 5.94 | (10.1)^{d} |
| | 3600 | 1.08 | (1.08-1.08) | 1010 | (12.5) | 12500 | (16.2) | 34.2 | (13.1) | 27100 | (20.7) | 0.132 | (20.7) | 6.56 | (16.4) |

AUC₀₋ₜ = area under the concentration-time curve from time 0 to the time t (28 d); AUC_{inf} = area under the concentration-time curve from time 0 extrapolated to infinity; Cₘₐₓ = maximum observed concentration; N = number of subjects; CV = coefficient of variation; t_{1/2} = half-life; tₘₐₓ = time Cₘₐₓ was observed; V_{z} = Apparent volume of distribution.
^{a}Median (min-max range).
^{b}Pharmacokinetic parameters for each dose group were calculated based on 6 subjects except where noted.
^{c}N = 2
^{d}N = 5
^{e}N = 4

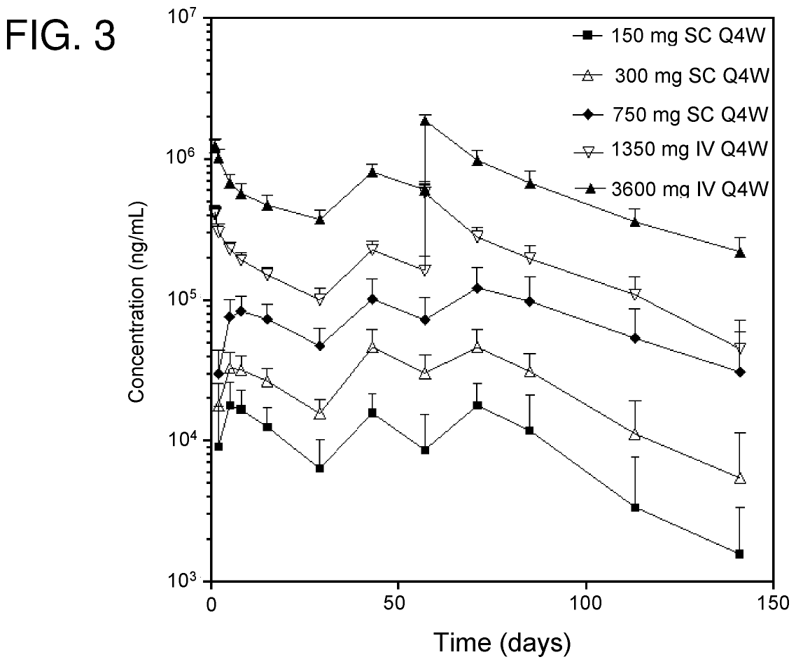

Based on the available interim data, PK data were obtained from healthy subjects who received MTPS9579A. Mean serum concentration of MTPS9579A versus time profiles from the SAD and MAD study were plotted in FIG. 2 and FIG. 3, respectively. The lower limit of quantification (LLOQ) was 250 ng/mL for the bioanalytical assay measuring MTPS9579A serum concentrations. Individuals with serum concentrations below LLOQ were excluded from NCA. PK parameter estimates are presented in Table 4 and Table 5 from the SAD and MAD portions of the GA40396 study, respectively.

Single-dose SC and IV PK data were available from 41 healthy subjects who received MTPS9579A. A review of the available SAD data demonstrated that absorption of MTPS9579A occurred with a median tₘₐₓ of 8 days after SC administration. From doses of 900 to 3600 mg IV, Cₘₐₓ and area under the concentration-time curve from Time 0 to the time t (28d) (AUCo₀₋ₜ) values increased dose proportionally, where a 4-fold increase in dose resulted in an approximately 4-fold increase in Cₘₐₓ and AUC₀₋ₜ. After single IV administration of 3600 mg, the highest doses administered in this study, Cₘₐₓ and AUCo₀₋ₜwere 1010 μg/mL and 12,500 μg-day/mL, respectively.

After a single SC administration of 30-300 mg MTPS9579A, the mean apparent clearance estimates ranged from 0.57-0.35 L/day. For single dose IV administration of 900-3600 mg MTPS9579A, the clearance values ranged from 0.19-0.13 L/day. Decreasing clearance estimates with increase in dose suggested a nonlinear PK, possibly due to target-mediated clearance at lower anti-tryptase serum concentrations (<15,000 ng/mL). After a single IV administration of 3600 mg of MTPS9579A, the mean terminal t_{1/2} was estimated to be 34 days by NCA and approximately matched the t_{1/2} projected by compartmental modeling analysis (˜30 days). It was not possible to estimate the bioavailability using NCA due to the observed nonlinearity in serum PK.

After multiple-dose SC or IV administration, PK data were evaluable from 40 healthy subjects who received MTPS9579A. PK parameters were limited from the MAD portion of the study because of insufficient serum concentration data. MTPS9579A exposure increased approximately dose proportionally when comparing the 1350 and 3600 mg IV 04W cohorts, where a 2.93- and 3.19-fold increase in Cₘₐₓ and AUCₜₐᵤ, respectively, was observed after the first dose administration. After the third and final dose administration of these cohorts (1350 mg IV and 3600 mg IV), a 3.29-fold increase in Cₘₐₓ was observed. Mean accumulation ratio (AR) after multiple SC Q4W doses ranged from 1.04-1.40 and was similar after multiple IV 04W dose cohorts (AR=1.36-1.53).

TABLE 5
Geometric Mean (CV %) Pharmacokinetic Parameter Estimates of MTPS9579A after First and
Last SC in Healthy Subjects in MAD (GA40396) (N = 8; All Dose Cohorts Unless Noted)

| | | First Dose | | | | | | Last Dose | | | | | |
|---|---|---|---|---|---|---|---|---|---|---|---|---|---|
| | Dosing | | | | | AUCₜₐᵤ | | | | | | AUCₜₐᵤ | AR |
| Dose/ | Interval | tₘₐₓ^{a} | | Cₘₐₓ | | (μg · | | tₘₐₓ^{a} | | Cₘₐₓ | | (μg · | C_{max, 3}/ |
| Regimen | (τ, day) | (day) | | (μg/mL) | | day/mL) | | (day) | | (μg/mL) | | day/mL) | C_{max, 1} |
| 150 mg SC | 28 | 5 | (5-8) | 16.7 | (46.4) | 301 | (48.2) | 71 | (71-71) | 164 | (48.2) | 271 (38.8) ^{b} | 1.04 (20.9) |
| Q4W | | | | | | | | | | | | | |
| 300 mg SC | 28 | 5 | (5-8) | 33.1 | (30.5) | 665 | (25.0) | 71 | (71-71) | 43.9 | (36.3) | 1720 (33.1) ^{b} | 1.32 (22.7) |
| Q4W | | | | | | | | | | | | | |
| 750 mg SC | 28 | 5 | (5-5) | 71.6 | (33.4) | 1750 | (33.4) | 71 | (71-71) | 114 | (42.1) | 5540 (56.3) | 1.40 (18.8) |
| Q4W | | | | | | | | | | | | | |
| 1350 mg IV | 28 | 1.06 | (1.02-1.08) | 426 | (10.1) | 4670 | (13.1) | 57.02 | (57.02-57.08) | 581 | (14.5) | NA | 1.36 (7.74) |
| Q4W | | | | | | | | | | | | | |
| 3600 mg IV | 28 | 1.08 | (1.02-1.08) | 1250 | (13.5) | 14900 | (16.5) | 57.02 | (57.02-57.08) | 1910 | (9.28) | NA | 1.53 (6.55) |
| Q4W | | | | | | | | | | | | | |

AR = accumulation ratio; AUCₜₐᵤ = area under the concentration-time curve during the dosing interval 0-28; Cₘₐₓ = maximum observed concentration; N = number of subjects; NA = not available due to limited PK availability; Q4W = every 4 weeks; CV = coefficient of variation; tₘₐₓ = time Cₘₐₓ was observed.
^{a}Median (min-max range).
^{b} N = 7

### Clinical Pharmacodynamics

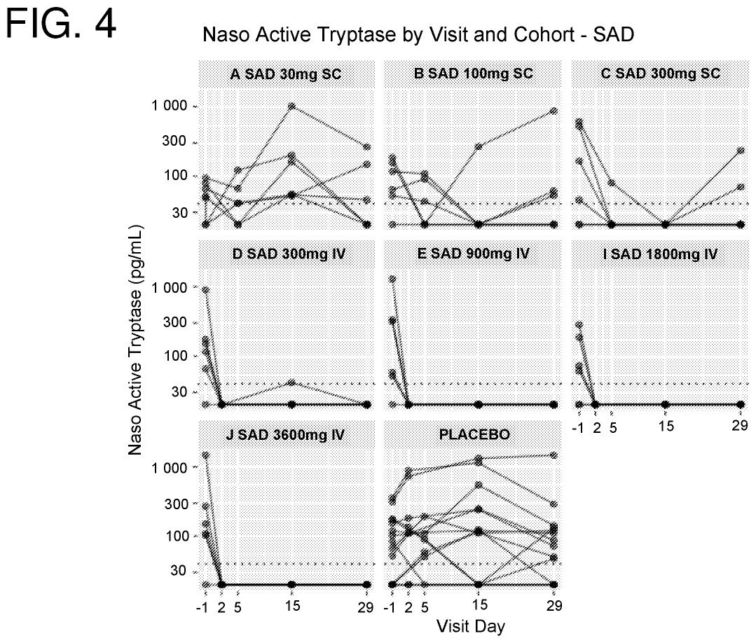

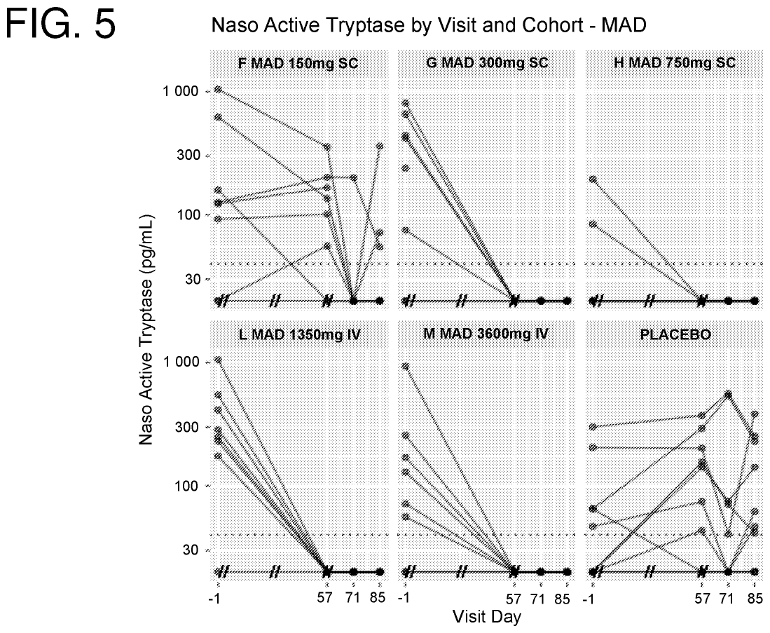

PD effects were assessed in all SAD and MAD cohorts following single or multiple doses of MTPS9579A or placebo administration SC or IV. Compared with the placebo group, MTPS9579A showed dose-dependent decreases in active tryptase levels in post-dose nasosorption samples, and tryptase activity dropped below the detection limit at doses 300 mg SC in healthy subjects (FIGS. 4 and 5). These data provide evidence that MTPS9579A is pharmacologically active and inhibits the target (active tryptase) in the upper airway of healthy volunteers.

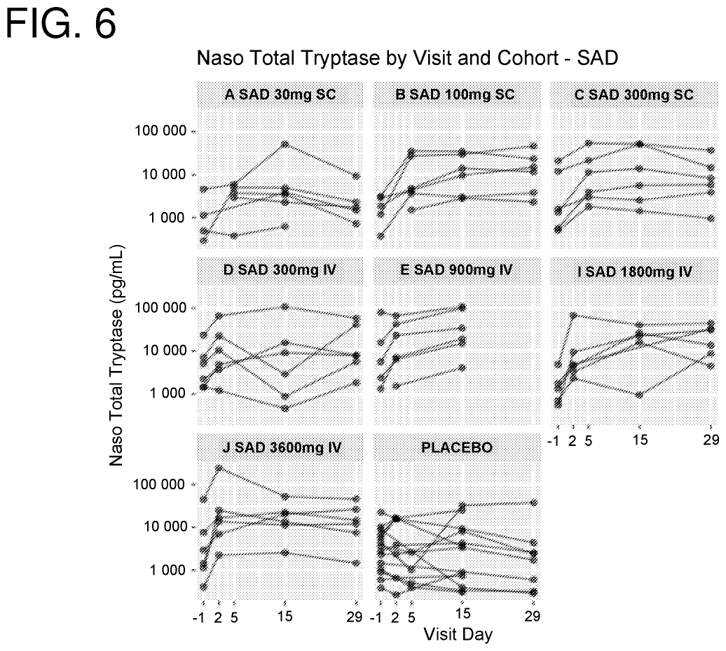

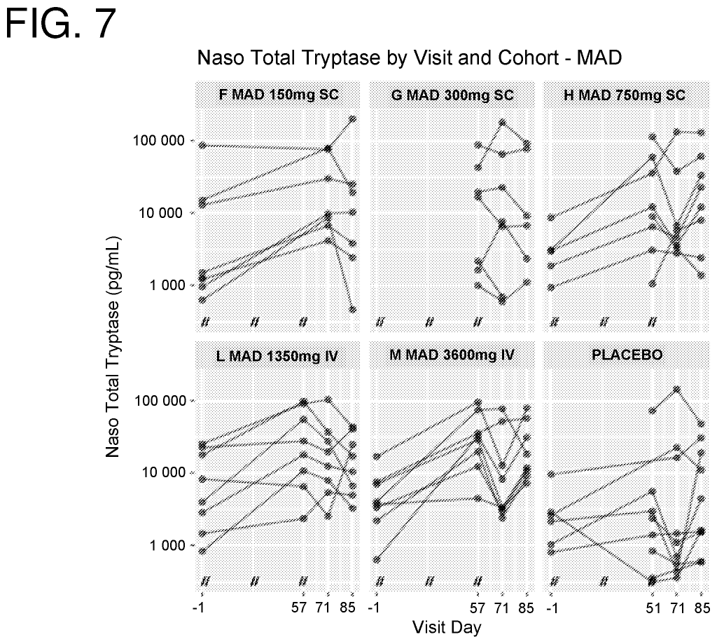

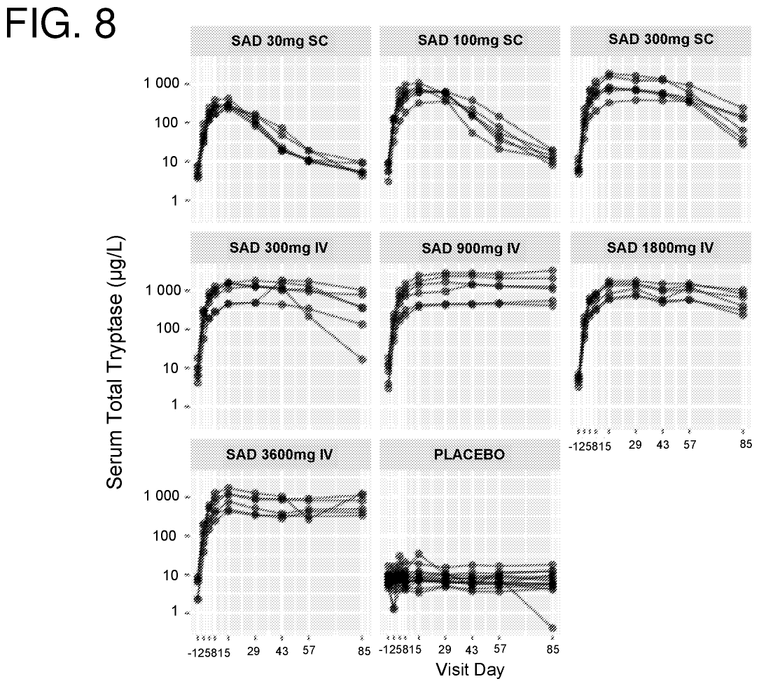

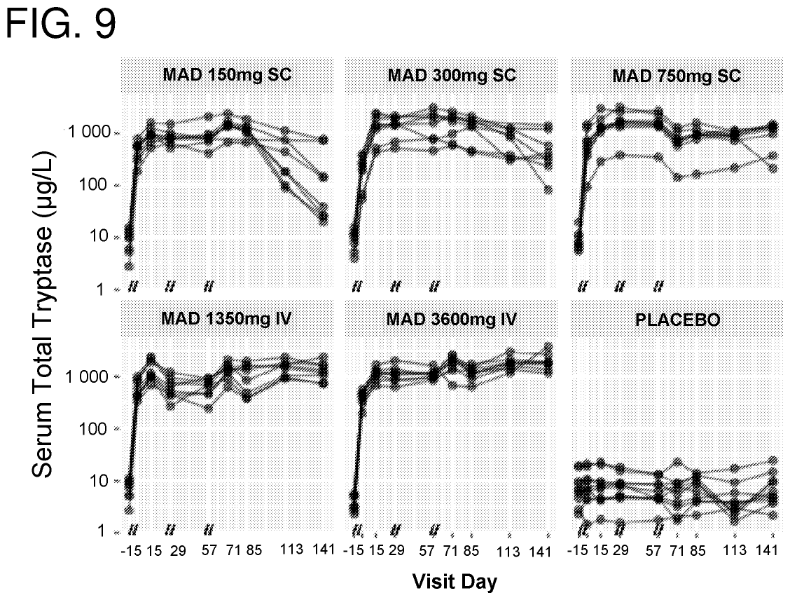

To further demonstrate that MTPS9579A can bind to tryptase in vivo, we measured serum and nasosorption levels of total tryptase at several timepoints post-dose. Levels of total tryptase in both nasosorption and serum increased over time in subjects who received MTPS9579A, reflecting antibody binding to tryptase and increasing its half-life (FIGS. 6-9). Total tryptase levels did not increase in the nasosorption or serum of those subjects who received placebo. Within each cohort, the peak post-dose total tryptase levels were variable across individuals and magnitude of increase appeared to correlate with baseline tryptase levels. In general, serum total tryptase levels elevated and plateaued below 4 mg/L. Elevated serum total tryptase of the low dose cohorts (i.e., Cohorts A-D) began to decrease in a dose-dependent manner. These data taken together provide evidence of target engagement for MTPS9579A in healthy volunteers.

B. Clinical Safety

As of the safety data cutoff date (Apr. 19, 2019), complete blinded data from Study GA40396 is available for all cohorts. These include single-dose cohorts of MTPS9579A (or matching placebo) of 30, 100, and 300 mg SC; 300, 900, 1800, and 3600 mg IV and multiple-dose cohorts of MTPS9579A (or matching placebo) of 150, 300, and 750 mg SC, as well as 1350 and 3600 mg IV 04W for three doses. As of the data cutoff date, 82 subjects (77%) reported a total of 412 AEs. There have been no deaths, no serious adverse events (SAEs), no reported WHO Grade 3 (severe) or higher AEs, and no reported AEs of special interest.

One subject in the MAD portion of the study was withdrawn from the study treatment because of a Grade 2 non-serious increase in blood CPK on Study Day 56. The subject, a 25-year-old male whose treatment assignment remains blinded, had a mildly elevated CPK at screening (264 U/L, upper limit of normal [ULN] 195 U/L) and prior to dosing (214 U/L). On Study Day 56, prior to the third administration of 150 mg of MTPS9579A or matching placebo, the subject was found to have a CPK of 1627 U/L (8.3xULN), and a Grade 2 AE of blood CPK elevation was reported and treatment was withdrawn. The AE was assessed by the investigator to be related to MTPS9579A/placebo. The subject did not have myalgias or muscle soreness. Five days later, on Study Day 61, CPK levels returned to 286 U/L, a near normal value that was comparable to the screening result and the AE was considered to have been resolved.

All AEs were WHO Grade 1 or 2; the highest grade AE was Grade 2 for 30 subjects (28%) and Grade 1 for 52 subjects (49%). A total of 72 subjects (68%) experienced an AE determined by the investigator to be related to MTPS9579A/placebo.

The most common AEs (seen in at least 5% of subjects, regardless of causality) were headache (32 subjects, 30%), injection site erythema (31 subjects, 29%), nasopharyngitis (18 subjects, 17%), injection site pallor (12 subjects, 11%), erythema (10 subjects, 9%), injection site bruising (6 subjects, 6%), back pain (6 subjects, 6%), and blood creatine phosphokinase increased (6 subjects, 6%). Although the event rate for injection site erythema was higher than anticipated, all events were mild, localized, and resolved without treatment within 1-3 hours after injection.

Based on the blinded safety data available as of 19 Apr. 2019, there are no safety concerns that affect the benefit-risk profile of the study drug, or that would preclude continued clinical development of MTPS9579A.

C. Immunogenicity

Prevalence of ADAs at baseline was defined as the proportion of the evaluable patient population in a study that was ADA positive at the baseline timepoint. Overall, the prevalence of ADA within GA40396 was 0% (0 out of 106). The incidence of ADAs (at post-baseline timepoints) was defined as the proportion of the study population found to have seroconverted (i.e., developed treatment-induced ADA's). The ADA incidence rate was 9.5% within the SAD portion of the study (4 out of 42) and 5% within the MAD portion of the study (2 out of 40).

D. Dosing Justification

The dose of MTPS9579A in a planned Phase Ila study (1800 mg IV 04W) was selected based on the totality of the following data: understanding of tryptase biology in healthy volunteers and patients with asthma, Phase la/b SAD/MAD clinical trial data, MTPS9579A properties, mechanism of action, nonclinical activity and safety, and prior external clinical experience targeting tryptase. The selected Phase Ila dose is within the dose range previously evaluated in the Phase la/b study (GA40396), which was well tolerated. In that study, doses up to 3600 mg of MTPS9579A were administered intravenously as a single dose or as a Q4W regimen (3 doses), which was the maximum dose tested. The dose and regimen to be tested in the Phase Ila study (1800 mg IV 04W) is projected to maximize the potential for clinical benefit based on the totality of data available to date. At steady-state trough concentrations, it is anticipated that this dose will reduce active tryptase levels by 95%, which accounts for the maximum concentration of active tryptase that may be present in the airway of patients with asthma while ensuring patient safety.

E. Conclusions

These data demonstrate that MTPS9579A can be safely dosed at relatively high doses, e.g., 1800 mg IV 04W and 3600 mg IV 04W. Moreover, the PD assessments in healthy subjects provide evidence that MTPS9579A is pharmacologically active and inhibits the target (active tryptase) in the upper airway. In view of these data, it is expected that the dosing regimens provided herein will be effective in treating asthma, including severe asthma that remains uncontrolled despite standard-of-care therapy.

### Example 3: Additional Results from Study GA40396

This Example describes results from the final data from the GA40396 study described in Examples 1 and 2. A total of 339 healthy male and female subjects were screened for this study. Of these, 166 subjects were enrolled and a total of 106 subjects were randomized to receive MTPS9579A or placebo. A total of 56 subjects were dosed in Part A (SAD; 42 subjects with MTPS9579A and 14 subjects with placebo) and 50 subjects were dosed in Part B (MAD; 40 subject with MTPS9579A and 10 subjects with placebo). All subjects who received at least 1 dose of study drug (MTPS9579A or placebo) comprised the safety population (N=106). Of these, 104 (98.1%) subjects completed the study.

As is described above, subjects were administered MTPS9579A or placebo, as follows: In Part A, subjects in Cohorts A, B, and C received a single SC 30 mg, 100 mg, and 300 mg dose of MTPS9579A or placebo, respectively on Day 1. Subjects in Cohorts D, E, I, and J received a single IV infusion 300 mg, 900 mg, 1800 mg, and 3600 mg dose of MTPS9579A or placebo, respectively, on Day 1.

In Part B, subjects received 3 doses (same dose level) in each cohort on Days 1, 29, and 57. Subjects in Cohorts F, G, and H received 150 mg, 300 mg, and 750 mg SC dose of MTPS9579A or placebo, respectively. Subjects in Cohorts L and M received 1800 mg and 3600 mg IV infusion dose of MTPS9579A or placebo, respectively.

1. Pharmacokinetic Results

A. Part A (SAD)

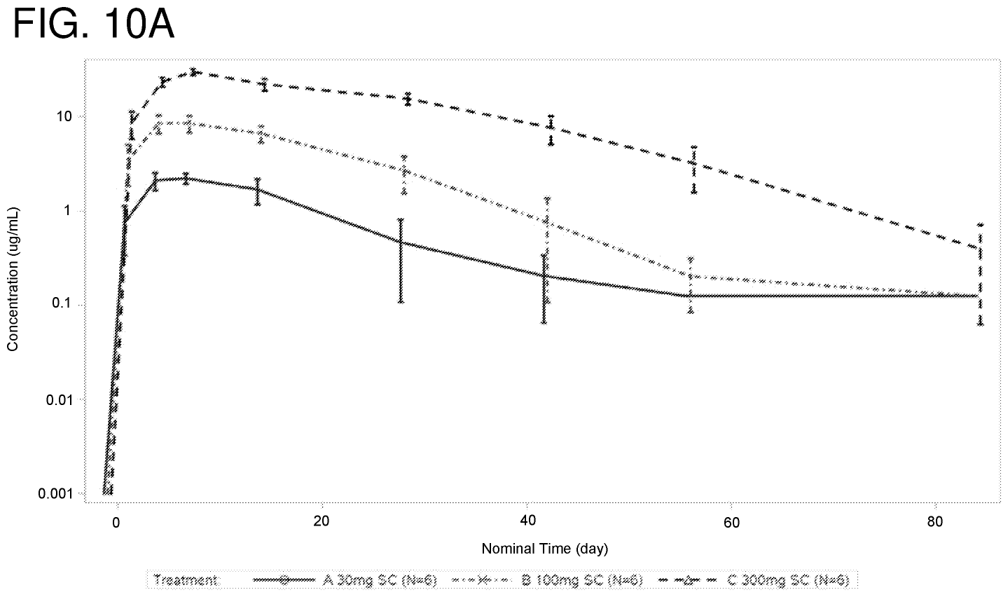

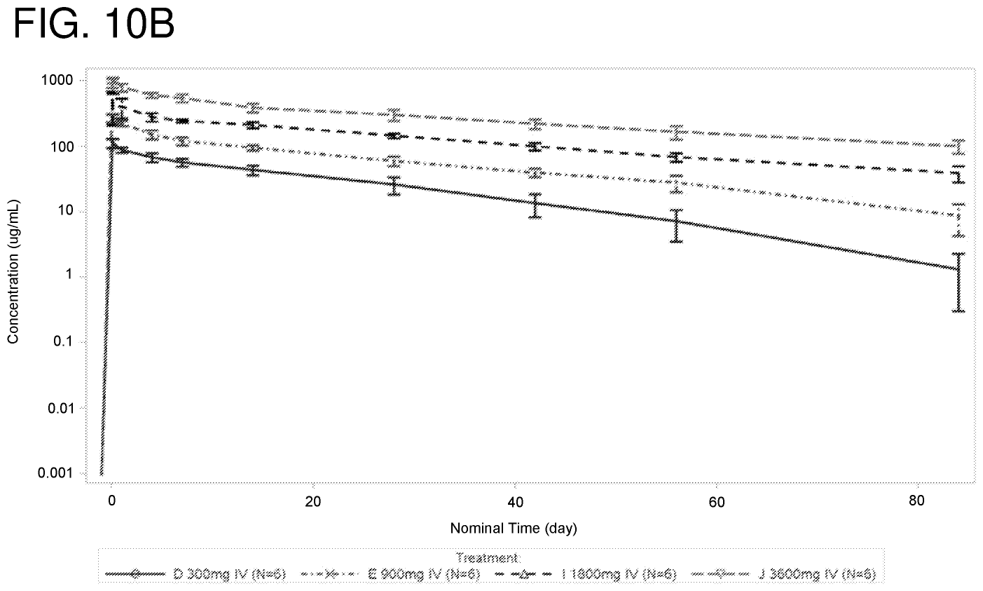

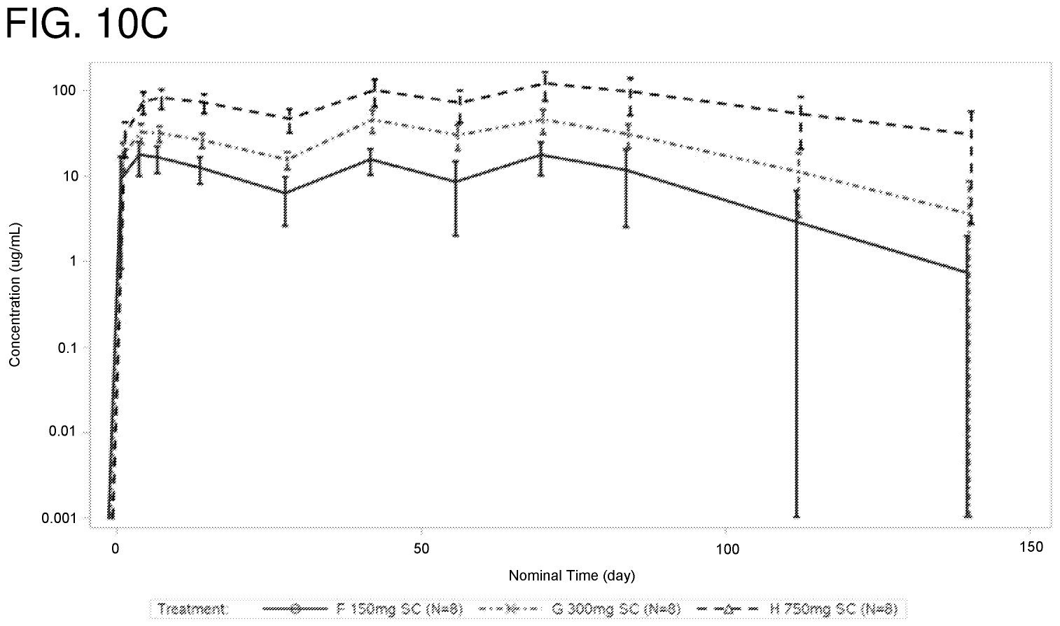

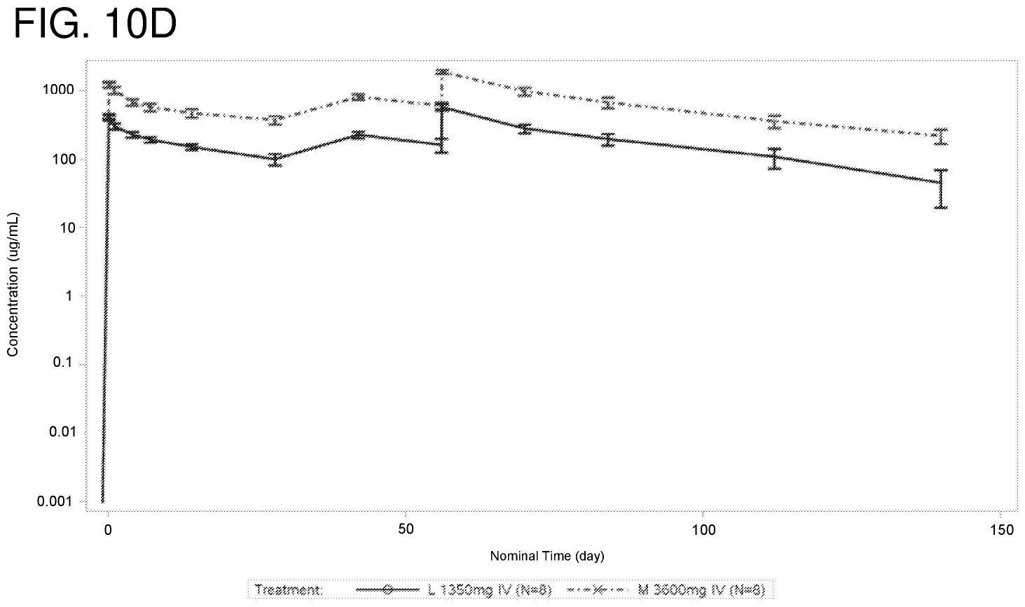

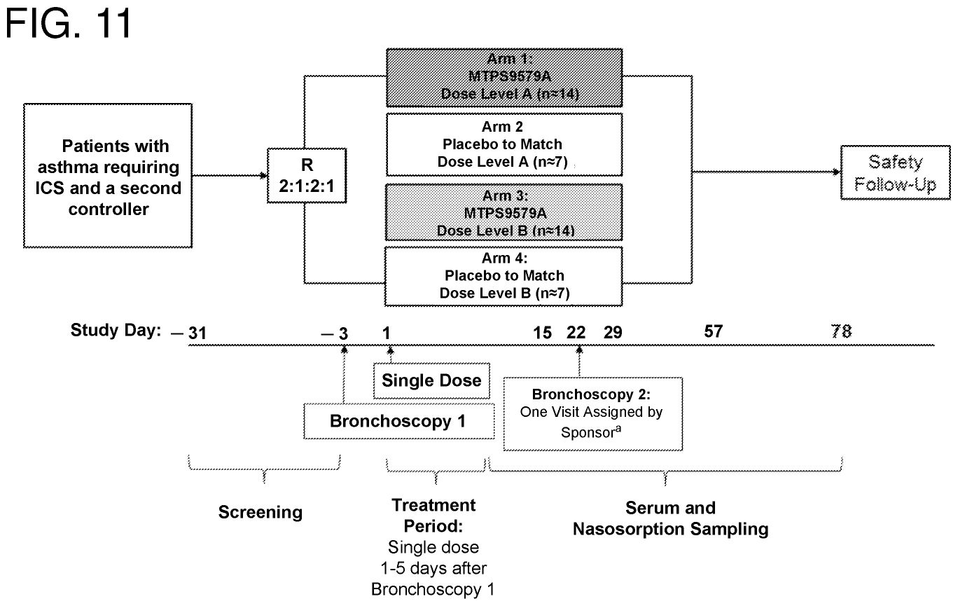

FIGS. 10A and 10B show mean serum MTPS9579A concentration over time for SC cohorts in the SAD portion of Study GA40396 (FIG. 10A) and for IV cohorts in the SAD portion of Study GA40396 (FIG. 10B).

Following a single administration of MTPS9579A to healthy subjects, Cₘₐₓ and AUC_{0-inf} increased with increasing MTPS9579A dose for IV doses. Tₘₐₓ was observed at same time for Cohorts D, E, I, and J regardless of dose levels (median Tₘₐₓ˜1.05 days). The mean _{t1/2} across the dose groups was approximately 24.3 days (mean (+/−SD) range from 11.8 (4.30) days at the 300 mg IV dose, increasing to 35.1 (5.60) days at the 3600 mg IV dose). Clearance values (CL) ranged from 140 (28.5) mL/day at the 3600 mg IV dose, increasing to 203 (45.6) mL/day at the 900 mg IV dose.

Volume of distribution (Vd) ranged from 2937 (896) mL at the 300 mg IV dose, increasing to 6929 (959) mL at the 3600 mg IV dose. Cₘₐₓ and AUCo_{0-inf} increased with increasing MTPS9579A dose for SC doses. Tₘₐₓ was observed at same time for Cohorts A, B, and C regardless of dose levels (median Tmax ˜6.9 days). The mean t_{1/2} across the dose groups was approximately 9.5 days (mean (+/−SD) range from 7.25 (0.737) days at the 100 mg SC dose, increasing to 11.3 (1.89) days at the 30 mg SC dose). Apparent clearance values (CL/F) ranged from 362 (55.5) mL/day at the 300 mg SC dose, increasing to 576 (42.7) mL/day at the 30 mg SC dose. Apparent volume of distribution (V/F) ranged from 5211 (696) mL at the 300 mg SC dose, increasing to 9300 (878) mL at the 30 mg SC dose.

Bioavailability: Clearance values after MTPS9579A ranged from 362 mL/day to 576 mL/day after single dose SC administration and from 140 mL/day to 203 mL/day after single dose IV administration. Decreasing clearance estimates with increase in dose suggest nonlinear PK, possibly due to target-mediated clearance at lower anti-tryptase serum concentrations. Due to the wide range of clearance values, it was not possible to estimate bioavailability using NCA with Cohort C and D.

B. Part B (MAD)

FIGS. 10C and 10D show mean serum MTPS9579A concentration over time for SC cohorts in the MAD portion of Study GA40396 (FIG. 10C) and for IV cohorts in the MAD portion of Study GA40396 (FIG. 10D).

Following Day 57 dose administration, Cₘₐₓ and AUC₀₋ₜₐᵤ increased with increasing MTPS9579A dose for SC doses. Tₘₐₓ were observed at same time for Cohorts F, G, and H regardless of dose levels (median Tₘₐₓ˜70.0 days). The mean t_{1/2} across the dose groups was approximately 19.5 days (mean (+/−SD) range from 11.2 (3.65) days at the 150 mg SC dose, increasing to 29.9 (14.6) days at the 750 mg SC dose). Following repeated dosing, the accumulation ratio ranged from 1.22 (0.153) at the 150 mg SC dose, increased to 2.11 (0.714) at the 750 mg SC dose AUC, suggesting weak to relatively modest accumulation across the cohorts.

Cₘₐₓ and AUC₀₋ₜₐᵤ increased with increasing MTPS9579A dose for IV doses. Tₘₐₓ was observed at same time for Cohorts L and M regardless of dose levels (median Tₘₐₓ˜56.1 days). The mean t_{1/2} across the dose groups was approximately 29.8 days (mean (+/−SD) range from 25.5 (6.31) days at the 1350 mg IV dose, increasing to 34.1 (4.82) days at the 3600 mg IV dose). Following repeated dosing, the accumulation ratio ranged from 1.88 (0.311) at the 1350 mg IV dose, increased to 2.30 (0.241) at the 3600 mg IV dose for AUC suggesting relatively modest accumulation across the cohorts.

2. Safety

In the combined SAD and MAD cohorts overall, a total of 413 treatment-emergent adverse events (TEAEs) were reported by 82 (77.4%) of the 106 subjects who received at least one dose of study medication (safety population): 339 TEAEs were reported by 63 (76.8%) of 82 subjects who received MTPS9579A and 74 TEAEs were reported by 19 (79.2%) of 24 subjects who received placebo. The SAD and MAD cohorts had similar frequencies and severities of TEAEs between cohorts and between volunteers on active drug and those on placebo. There were no deaths, serious or life threatening adverse events (AEs) during the study. Only 1 subject discontinued due to a TEAE (blood creatine phosphokinase increased) during Part B (MAD). This indicates that the study drug dose-levels were well tolerated, with no safety concerns.

In SAD cohorts, the TEAE rate and severity was comparable across the dose-level cohorts, between subjects who received MTPS9579A or placebo. The majority of observed TEAEs were Grade 1 in severity and judged as not related to the study drug. In MAD cohorts, the TEAE rate and severity was comparable across the dose-level cohorts, between subjects who received MTPS9579A or placebo. The majority of observed TEAEs were Grade 1 in severity and judged as not related to the study drug.

A total of 7 subjects (4 subjects in Part A (SAD) and 3 subjects in Part B (MAD)) had 8 clinically significant laboratory abnormalities during the study that led to TEAEs. The majority of these laboratory abnormalities were increased creatine phosphokinase. All these TEAEs were Grade 1 in severity and resolved by the end of the study. There were no pattern in the development or magnitude of laboratory abnormalities between cohorts or between volunteers who received MTPS9579A or placebo. Three (3) vital signs abnormalities (1 in Part A (SAD) and 2 in Part B 9MAD)) observed during the scheduled measurements were considered to be TEAEs. All of these were Grade 1 in severity and resolved by the end of the study.

No clinically significant abnormalities were observed with respect to ECG results. No relevant differences were observed between the treatment groups with respect to mean values and changes from baseline for clinical laboratory results, vital signs, and ECG results.

Overall, 7 subjects (4 in Part A and 2 in Part B, who received active drug and 1 subject in placebo cohort of Part A) developed an ADA response following the study drug administration. However, no safety concerns were expected regarding immunogenicity.

In summary, the administration of MTPS9579A was well tolerated in healthy subjects when administered as either a single SC or IV dose over the range of 30 mg to 3600 mg and following multiple SC or IV dose Q4W from 150 mg to 3600 mg.

3. Biomarker Assessments

A. Part A (SAD)

The absolute values for active tryptase reported after MTPS9579A administration on Days 2, 5, 15, and 29 for all subjects who received MTPS9579A fell below the lower limit of quantification (LLOQ) for cohorts dosed at 300 mg SC. The absolute values for active tryptase reported on Days -1, 2, 5, 15, and 29 for subjects who received placebo remained generally unchanged.

B. Part B (MAD)

The absolute values for active tryptase reported after MTPS9579A administration on Days 57, 71, and 85 for all subjects who received MTPS9579A fell below LLOQ for cohorts dosed at 300 mg SC. The absolute values for active tryptase reported on Days -1, 57, 71, and 85 for subjects who received placebo were generally unchanged.

4. Conclusions

A total of 82 subjects (42 subjects in Part A (SAD) and 40 subjects in Part B (MAD)) were treated with MTPS9579A.

Overall, MTPS9579A was well tolerated in healthy subjects when administered as a single SC or

IV dose of MTPS9579A over the range of 30 mg to 3600 mg.

None of the TEAEs reported were severe or serious, and the majority was mild in intensity.

The incidence of subjects who reported TEAEs was generally comparable across all the cohorts for both SAD and MAD parts and no trends were observed between the cohorts. No safety concerns with respect to the clinical laboratory tests, ECGs, and vital signs were raised.

Systemic exposure (Cₘₐₓ and AUC) increased with increasing dose of MTPS9579A.

Following repeated dosing of MTPS9579A, the accumulation ratio obtained for AUC suggests weak to relatively modest accumulation across the SC cohorts and a relatively modest accumulation across the IV cohorts.

Decreasing clearance estimates with increase in dose suggested nonlinear PK, possibly due to target-mediated clearance at lower anti-tryptase serum concentrations.

Due to the wide range of clearance values, it was not possible to estimate bioavailability using NCA for MTPS9579A.

Overall, 7 subjects (5 in Part A and 2 in Part B) developed an ADA response following the study drug administration and 2 subjects reported treatment-induced ADA in all experimental subjects.

Overall, MTPS9579A administered as a single dose and multiple ascending doses showed dose-dependent decrease in active tryptase levels in the upper airways of healthy volunteers in post-dose nasosorption samples when compared to subjects treated with placebo.

### Example 4: A Phase Ic, Multicenter, Randomized, Observer-Blinded, Placebo-Controlled Study to Evaluate the Safety, Tolerability, Pharmacokinetics, and Pharmacodynamic Effects of a Single Dose of MTPS9597A in Patients with Asthma Requiring Inhaled Corticosteroids and a Second Controller

1. Objectives and Endpoints

This study will evaluate the safety, pharmacokinetics, pharmacodynamics, immunogenicity, and activity of a single dose of MTPS9579A in patients with asthma requiring inhaled corticosteroids (ICS) and a second controller. Specific objectives and corresponding endpoints for the study are outlined below. FIG. 11 presents an overview of the study design.

A. Safety Objectives

The safety objective for this study is to evaluate the safety and tolerability of MTPS9579A on the basis of the following endpoints:

Incidence and severity of adverse events, with severity determined according to the WHO toxicity scale

Change from baseline in targeted vital signs

Change from baseline in targeted clinical laboratory test results

Change from baseline in ECG parameters

B. Pharmacokinetic Objectives

The pharmacokinetic (PK) objective for this study is to characterize the serum PK profile of MTPS9579A on the basis of the following endpoint:

Serum concentration of MTPS9579A at specified timepoints

The exploratory PK objectives for this study are as follows:

To evaluate potential relationships between drug exposure and the safety of MTPS9579A on the basis of the following endpoint:
Relationship between serum concentration or PK parameters for MTPS9579A and safety endpoints

To characterize the MTPS9579A PK profile in nasal mucosal lining fluid and bronchial mucosal lining fluid on the basis of the following endpoint:
Concentration of MTPS9579A in nasal mucosal lining fluid and bronchial mucosal lining fluid at specified timepoints

C. Activity Objectives

The activity objective for this study is to provide evidence of MTPS9579A activity in the lower airway on the basis of the following endpoint:

Relative change from baseline in active tryptase and total tryptase levels in bronchial mucosal lining fluid samples at specified timepoints

The exploratory activity objective for this study is to provide evidence of MTPS9579A activity in the upper airway on the basis of the following endpoint:

Relative change from baseline in active tryptase and total tryptase levels in nasal mucosal lining fluid samples at specified timepoints

D. Immunogenicity Objectives

The immunogenicity objective for this study is to evaluate the immune response to MTPS9579A on the basis of the following endpoint:

Prevalence of anti-drug antibodies (ADAs) at baseline and incidence of ADAs during the study

The exploratory immunogenicity objective for this study is to evaluate potential effects of ADAs on the basis of the following endpoint:

Relationship between ADA status and PK, safety, activity, and biomarker endpoints

E. Biomarker Objective

The exploratory biomarker objective for this study is to identify biomarkers that can provide evidence of MTPS9579A activity (i.e., pharmacodynamic (PD) biomarkers) based on the following endpoints:

Relative change from baseline in biomarker levels in nasal mucosal lining fluid, bronchial mucosal lining fluid, endobronchial biopsy, epithelial brushing, urine, and blood samples

Relationship among biomarker levels in nasal mucosal lining fluid, bronchial mucosal lining fluid, endobronchial biopsy, epithelial brushing, urine, and blood samples

Relationship between biomarkers in tissue, urine, and blood and PK endpoints

2. Study Design

A. Description of Study

This is a Phase Ic, multicenter, randomized, observer-blinded, placebo-controlled study to evaluate the safety, tolerability, pharmacokinetics, and PD effects of a single dose of MTPS9579A in patients with asthma requiring ICS and a second controller. This study will be conducted at up to approximately 8 experienced bronchoscopy sites located throughout the United Kingdom. Approximately 42 patients will be enrolled and randomized in the study, with approximately 28 patients receiving

MTPS9579A and approximately 14 patients receiving matching placebo. Patients who do not complete the study may be replaced at the Sponsor's discretion.

Patients will be screened and randomized into one of four study arms in a 2:1:2:1 ratio: active MTPS9579A (Dose Level A, 1800 mg) administered IV, placebo to match Dose Level A, active MTPS9579A (Dose Level B, 300 mg) administered IV, and placebo to match Dose Level B. After appropriate safety screening, patients will undergo a baseline bronchoscopy assessment with biomarker sampling consisting of an endobronchial biopsy, epithelial brushing, and bronchosorption sampling. Patients will also undergo a baseline nasosorption sampling procedure, as well as blood and urine collection. One to five days after the first bronchoscopy assessment, patients will receive study drug treatment. Treatment will consist of one IV dose of study drug (active MTPS9579A or placebo matching active MTPS9579A) on Day 1.

Each patient will undergo only one baseline and one follow-up bronchoscopy procedure. Patients will return for a follow-up bronchoscopy visit 3 weeks following dose administration on Day 1. Biomarker sampling at the follow-up bronchoscopy visit will include collection of endobronchial biopsies, epithelial brushings, bronchosorption, urine, nasosorption, and serum samples. There will be periodic assessments for safety monitoring and collection of blood and nasosorption samples. Assessments will end on approximately Day 78 with a safety follow-up visit. The Sponsor will review PK, PD, and available safety data at regular intervals, each to be triggered after 12-15 patients have undergone the follow-up bronchoscopy. Upon review of the data, the Sponsor may choose to change one or more of the study drug arms to an SC route of administration, with a corresponding change to one or more of the placebo arms with a matching placebo. In addition, the Sponsor may choose to change the nominal dose in Dose Level A and/or Dose Level B. The follow-up bronchoscopy visit timing may be updated by the Sponsor based on available PK/PD data but will not occur sooner than 1 week after the baseline bronchoscopy visit to allow for adequate recovery after the baseline bronchoscopy procedure. The flexible timing of the follow-up bronchoscopy is intended to enable the potential to characterize airway pharmacodynamics throughout the PK profile of MTPS9579A.

Additionally, at the Sponsor's discretion, upon review of data, approximately 14 additional patients may be enrolled and randomized to receive MTPS9579A or placebo.

B. Number of Patients

Approximately 42 male and female patients with asthma requiring ICS and a second controller between the ages of 18 and 65 years, inclusive, will be enrolled and randomized at up to approximately 8 investigative sites located in the United Kingdom.

3. Target Population

A. Inclusion Criteria

Patients must meet the following criteria for study entry:

Ability to comply with the study protocol, in the investigator's judgment

Age 18-65 years, inclusive, at the time of signing the Informed Consent Form

Body mass index of 18-35 kg/m² and weight ≥40 kg at screening

Asthma, confirmed by evidence of variable airflow obstruction or hyper responsiveness within 12 months of study entry via one or more of the following criteria:
Forced expiratory volume in 1 second (FEV1)/forced vital capacity (FVC) <70% with FEV1 variability ≥12% spontaneously (e.g., between clinic visits), or in response to oral corticosteroids
Concentration of methacholine needed to produce a 20% decrease in FEV1 from baseline (methacholine PC20)≤8 mg/mL (documented history of)
FEV1 bronchodilator response ≥12% and ≥200 mL with up to 400 mcg albuterol hydrofluoroalkane or 2.5-5 mg nebulized salbutamol

Asthma controller therapy: daily ICS and at least a second controller (LABA, LAMA,LTRA) for ≥3 months prior to screening, with no changes within 4 weeks prior to screening or during the screening period and no anticipated changes in controller dosing regimens throughout the study

Pre-bronchodilator FEV1≥50% predicted at screening

Asthma control questionnaire (ACQ-5)≥0.75 at screening

For women of childbearing potential: agreement to remain abstinent (refrain from heterosexual intercourse) or use contraceptive measures, and agreement to refrain from donating eggs

For men: agreement to remain abstinent (refrain from heterosexual intercourse) or use a condom, and agreement to refrain from donating sperm

B. Exclusion Criteria

Patients who meet any of the following criteria will be excluded from study entry:

Pregnant or breastfeeding, or intending to become pregnant during the study or within 110 days after the final dose of MTPS9579A.

Women of childbearing potential must have a negative serum pregnancy test at screening and a negative urine pregnancy test on Day 1.

Diagnosis of vocal cord dysfunction, reactive airways dysfunction syndrome, hyperventilation associated with panic attacks, or other mimics of asthma Diagnosis of occupational asthma, aspirin-sensitive asthma (if on chronic aspirin therapy within 2 weeks prior to screening or anticipated need of chronic aspirin therapy during the course of the study), asthma-chronic obstructive pulmonary disease (COPD) overlap syndrome, or bronchiolitis, as determined by the investigator

History of interstitial lung disease, COPD (fixed airflow obstruction due to asthma is an exception), or other clinically significant lung disease other than asthma

Current smoker, defined as someone who has smoked at least one cigarette per day (or pipe, cigar, “vaping” or marijuana) for ≥30 days within the 24 months prior to Day 1
A patient who smokes occasionally (i.e., at least one cigarette per day (or pipe, cigar, or marijuana) for 30 days within the 24 months prior to Day 1) and has a total smoking history of 10 pack-years may be permitted but must agree to abstain from all smoking during the study and must have abstained from any inhaled or marijuana products within the last 6 months.
   A pack-year is defined as the average number of packs of cigarettes per day times the number of years of smoking.

Former smoker with smoking history of >10 pack-years

Planned procedure (other than scheduled bronchoscopy for this study) or surgery during the study

Positive test for tuberculosis (TB) during screening, defined as a positive QUANTIFERON® test (QFT)

Patients with a history of Bacille Calmette-Guérin (BCG) vaccination should be screened using the QFT only; the following criteria for the QFT apply:
An indeterminate QFT should be repeated
A positive QFT or two successive indeterminate QFT results should be considered a positive diagnostic TB test
An indeterminate QFT followed by a negative QFT test, should be considered a negative diagnostic TB test
Patients with a positive QFT (see criteria above) are eligible if they meet all of the following criteria:
   No symptoms consistent with TB
   Documented history of a completed course of adequate prophylaxis (completed treatment for latent TB per the treatment options as stated in the WHO guideline) prior to screening
   No known exposure to a case of active TB after most recent prophylaxis
   No evidence of active TB on chest radiograph within 3 months prior to screening

History of anaphylaxis to any biologic therapy for any indication

History of documented immune complex disease (Type III hypersensitivity reactions) to monoclonal antibody administration

Known sensitivity to any of the active substances or their excipients to be administered during dosing

History of any known immunodeficiency disorder, including but not limited to HIV infection

Intubation for respiratory failure due to asthma within 12 months prior to screening

Initiation of or change in allergen immunotherapy for any disease within 3 months prior to screening or during the screening period

Treatment with phosphodiesterase-4 inhibitors (e.g., roflumilast) within 4 weeks prior to screening or during the screening period, or anticipated need for phosphodiesterase-4inhibitors during the course of the study

Treatment with maintenance oral or SC β-agonist therapy (e.g., terbutaline) within 2 weeks prior to screening and during the screening period

Treatment with immunomodulatory, immunosuppressive (e.g., methotrexate, troleandomycin, oral gold, cyclosporine, azathioprine), or experimental anti-inflammatory therapy within 3 months or 5 drug half-lives prior to screening (whichever is longer) or during the screening period, or anticipated need for these medications during the course of the study

Treatment with maintenance oral or inhaled antibiotics within 2 weeks prior to screening and during the screening period

Treatment with β-blocking agents (topical, oral, or other systemic) within 2 weeks prior to screening and during the screening period

Treatment with homeopathic medications, herbal medications intended for the treatment of asthma, acupuncture, or hypnosis therapy within 2 weeks prior to screening, and during the screening period

Treatment with a licensed biologic agent (e.g., omalizumab, mepolizumab, or suplatast) within 3 months or 5 drug half-lives prior to screening (whichever is longer) or during the screening period

Treatment with any investigational therapy within 3 months or 5 drug half-lives prior to screening (whichever is longer) or during the screening period

Maintenance oral corticosteroid therapy, defined as daily or alternate-day oral corticosteroid maintenance therapy within 3 months prior to screening

Treatment with systemic (oral, IV, or intramuscular (IMO) corticosteroids within 4 weeks (or 12 weeks for IM) prior to screening or during the screening period for any reason, including an acute exacerbation event (pulse treatment)

Treatment with intra-articular corticosteroids within 4 weeks prior to screening or during the screening period, or anticipated need for intra-articular corticosteroids during the course of the study

Maintenance intermittent positive pressure ventilation physiotherapy within 2 weeks prior to screening or during the screening period, or anticipated need during the course of the study

Maintenance bilevel positive airway pressure therapy within 2 weeks prior to screening or during the screening period, or anticipated need during the course of the study

History of bronchial thermoplasty treatment prior to screening or during the screening period, or anticipated need during the course of the study

Serious infection requiring oral or IV antibiotics within 28 days prior to screening

Treatment with immunoglobulin or blood products within 4 weeks prior to screening or during the screening period, or anticipated need for immunoglobulin or blood products during the course of the study

Treatment with any live or live, attenuated vaccines within 4 weeks prior to screening or during the screening period, or anticipated need for live, attenuated vaccines during the course of the study

Illicit drug or alcohol abuse within 12 months prior to screening, in the investigator's judgment

Poor peripheral venous access

Any serious medical condition or abnormality in clinical laboratory tests that, in the investigator's judgment, precludes the patients safe participation in and completion of the study

History of malignancy, except for appropriately treated carcinoma in situ of the cervix, nonmelanoma skin carcinoma, or Stage I uterine cancer

Donation or loss of blood (excluding the volume of blood that will be drawn during screening procedures) as follows: 50-499 mL of blood within 30 days or >499 mL of blood within 56 days prior to study drug administration

Unable to safely undergo elective flexible bronchoscopy because of any one of the following:
History of allergic reactions to local anesthetics (e.g., lidocaine) to be used in the procedure
Presence of clinically significant abnormality on screening coagulation tests
Presence of clinically important comorbidities (e.g., uncontrolled diabetes, uncontrolled coronary artery disease, acute or chronic renal failure, and uncontrolled hypertension) that, in the opinion of the investigator, may make the patient unsuitable for elective bronchoscopy

Treatment with aspirin, anticoagulant (e.g., warfarin), or antiplatelet (e.g., clopidogrel) medication within 7 days before the first or second bronchoscopy

Use of non-steroidal anti-inflammatory drugs (NSAIDs) is allowed.

History or presence of an abnormal ECG that is clinically significant in the investigator's opinion, including complete left bundle branch block, second- or third degree atrioventricular heart block, or evidence of prior myocardial infarction

QT interval corrected through use of Fridericia's formula (QTcF)>450 ms, if patient is male, or QTcF>470, if patient is female, demonstrated by at least two ECGs>30 minutes apart

Current treatment with medications that are well known to prolong the QT interval 4. End of Study The end of this study is defined as the date when the last patient, last visit occurs or safety follow-up is performed for the last patient, whichever occurs later. The end of the study is expected to occur approximately 3 months after the last patient is randomized.

5. Length of Study

The total length of the study, from screening of the first patient to the end of the study, is expected to be approximately 16 months.

6. Investigational Medicinal Products

The investigational medicinal product (IMP) for this study is MTPS9579A.

7. Test Product (Investigational Drug) and Comparator

Patients will be screened and randomized into one of four study arms in a 2:1:2:1 ratio: active MTPS9579A (Dose Level A, 1800 mg) administered IV, placebo to match Dose Level A, active MTPS9579A (Dose Level B, 300 mg) administered IV, and placebo to match Dose Level B. One to five days after the first bronchoscopy assessment, patients will receive study drug treatment. Treatment will consist of one IV dose of study drug (active MTPS9579A or matching placebo) on Day 1.

8. Statistical Methods

A. Primary Analysis The primary objective of this study is to characterize the safety profile associated with MTPS9579A. Statistical summaries will be descriptive in nature (e.g., incidence rates, means, and percentiles). The main activity objective of this study is to characterize the PK/PD profile of MTPS9579A. The primary PD outcome will be the relative change in active and total tryptase levels in bronchial mucosal lining fluid from baseline at the bronchosorption follow-up visit.

B. Determination of Sample Size

A total of approximately 42 patients will be randomized into four arms in a 2:1:2:1 ratio of MTPS9579A Dose Level A, matching placebo for Dose Level A, MTPS9579A Dose Level B, and matching placebo for Dose Level B. This sample size provides 80% power to detect a 50% change in active tryptase levels in bronchial mucosal lining fluid compared to baseline. The calculations assume a two-sided significance level of 0.05 and are based on mean log biomarker levels of 9.2 and a standard deviation of 1, which is derived from internal study data (Study GB29260) from total tryptase mucosal lining fluid. The targeted effect size has been chosen based on internal decision-making criteria. However, the primary goal is estimation rather than hypothesis testing, in order to characterize the drug's mode of action and level of activity in the desired population in the lower airways.

There is currently no available data on active tryptase levels using bronchosorption methods in patients with asthma. Therefore, at least one cumulative data review will be conducted to better understand variability and levels of active tryptase in the lower airways. For example, if cumulative review data indicate near complete inhibition of active tryptase levels, lower doses may be evaluated to better characterize PK/PD and aid in dose selection. Likewise, if cumulative review data are more variable than assumed, the sample size may be increased to improve precision around estimated effect size.

C. Periodic Data Review

Periodic review of data will be conducted to evaluate the PK/PD profile of MTPS9579A in systemic circulation as well as in the upper and lower airways (nasal and bronchial mucosa).

All available PK/PD and safety data will be reviewed after every 12-15 patients have undergone follow-up bronchoscopy. The analysis will be performed and interpreted by Sponsor study team personnel, who will have full access to unblinded data. Access to treatment assignment information will follow the Sponsor's standard procedures.

The actions that may be taken following cumulative data review include: 1. Dose and/or route of administration changes 2. Enrollment of additional patients (up to 14 additional patients) 3. The follow-up bronchoscopy visit timing may be updated to 2 or 4 weeks after dosing (for IV administration) or 1, 4, or 5 weeks after dosing (for SC administration).

9. Study and Dose Rationale

The rationale for conducting this Phase Ic bronchoscopy study of MTPS9579A in patients with asthma requiring ICS and a second controller is to evaluate pharmacokinetics and organ-specific pharmacodynamics (target inhibition in lung) in a relevant patient population. The goals of this study are to determine tryptase concentrations and MTPS9579A levels in nasal mucosal lining fluid and bronchial mucosal lining fluid, understand PK/PD relationships of MTPS9579A and tryptase in relevant tissues, and guide dose selection for a dose-ranging Phase Iib study of MTPS9579A.

To further assess the PK characteristics and extent of target inhibition of MTPS9579A in this population of patients with asthma, two MTPS9579A arms and two placebo arms will be evaluated for this Phase Ic study. The doses administered in this study will be determined by the Sponsor to best characterize the safety, PK, and PD activity of MTPS9579A but will not exceed exposures that have been deemed well tolerated in the ongoing Phase I SAD/MAD study in healthy volunteers (Study GA40396).

Patients in Arm 1 will be administered a single dose of 1800 mg IV MTPS9579A (Dose Level A). Patients in Arm 3 will be administered a single dose of 300 mg IV MTPS9579A (Dose Level B). These doses may be increased or decreased based on emerging data but will not exceed 3600 mg IV. Patients in Arms 2 and 4 will receive a single dose of placebo to match the dose in Arms 1 and 3, respectively. Upon review of preliminary data, the Sponsor may change the dose of active drug in Arm 1 or 3. The Sponsor may also change to SC drug administration due to potential advantages for future development including ease of administration, length of action, and potential for a reduced magnitude of fluctuation in plasma drug concentrations. At no point will the study drug dose exceed 3600 mg SC.

The proposed dose range for MTPS9579A was selected based on the totality of the data, including understanding of tryptase biology in healthy volunteers and patients with asthma, MTPS9579A properties, mechanism of action, nonclinical activity and safety, and prior clinical experience targeting tryptase. Projections on the dose and exposure needed for clinical efficacy will take into account the maximum concentration of active tryptase that may be present in the airway of patients with asthma. Dose selection will be based on ongoing analysis of safety, PK, and PD data from the currently described study.

The selected range of doses in this study is within the dose range previously evaluated in healthy volunteers. In Study GA40396, an ongoing SAD/MAD study, single doses of up to 3600 mg of MTPS9579A were administered IV and single doses of up to 750 mg were administered SC.

### Example 5: A Phase Ila, Multicenter, Randomized, Placebo-Controlled, Double-Blind Study to Evaluate the Efficacy, Safety, and Pharmacokinetics of MTPS9579A in Patients with Asthma Requiring Inhaled Corticosteroids and a Second Controller

1. Objectives and Endpoints

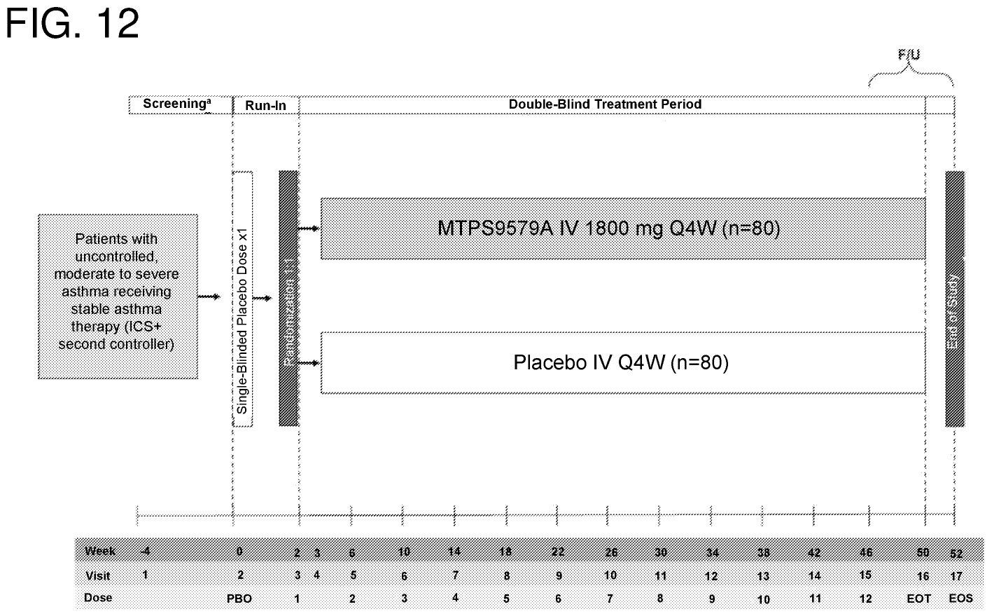

This study will evaluate the efficacy, safety, and pharmacokinetics of MTPS9579A compared with placebo in patients with uncontrolled asthma despite the use of inhaled corticosteroids (ICS) and a second controller. Specific objectives and corresponding endpoints for the study are outlined below. FIG. 12 presents an overview of the study design.

A. Primary Efficacy Objective

The primary efficacy objective for this study is to evaluate the efficacy of MTPS9579A compared with placebo on the basis of the following endpoint:

Time to first CompEx event, a composite endpoint defined as time from randomization to first asthma exacerbation or diary worsening during the 48-week double-blind treatment period (from the randomization visit (Week 2) to end of treatment (Week 50)). See Fuhlbrigge et al. Lancet 5(7):577-590, 2017 for additional information on the CompEx endpoint. Asthma exacerbations and diary worsening are defined as follows:
Asthma exacerbations are assessed by the investigator and defined as new or increased asthma symptoms (wheezing, coughing, dyspnea, chest tightness, and/or nighttime awakenings due to these symptoms) that result in one or both of the following:
   Hospitalization or an emergency department or urgent care visit requiring administration of systemic corticosteroid treatment
   Treatment with systemic (IV, intramuscular (IM), or oral) corticosteroids for 3 days or a long-acting depot corticosteroid preparation with a therapeutic effectiveness of 3 days

Diary worsening is based on the occurrence of prespecified changes (deteriorations) in a subset of the following six parameters: morning peak expiratory flow rate (PEFR), evening PEFR, morning symptom score, evening symptom score, morning short-acting rescue therapy use, and evening short-acting rescue therapy use.

B. Secondary Efficacy Objective

The secondary efficacy objective for this study is to evaluate the efficacy of MTPS9579A compared with placebo on the basis of the following endpoints:

Rate of asthma exacerbations (as defined in primary efficacy objective and assessed by the investigator) during the 48-week double-blind treatment period

Time to first asthma exacerbation during the 48-week double-blind treatment period

Absolute and relative change from randomization in pre-bronchodilator forced expiratory volume in 1 second (FEV₁; liters) at Week 50

Absolute and relative change from randomization in fractional exhaled nitric oxide (FeNO) at Week 50

C. Exploratory Efficacy Objective

The exploratory efficacy objective for this study is to evaluate the efficacy of MTPS9579A compared with placebo on the basis of the following endpoints:

Rate of severe asthma exacerbations during the 48-week double-blind treatment period, defined as asthma symptoms requiring hospitalization or resulting in death attributed to asthma

Absolute change from randomization in pre-bronchodilator FEV, (percentage predicted) at Week 50

Absolute change from randomization in patient-reported daytime asthma symptom severity, as measured by a daily symptom diary (as defined in primary efficacy objective), at Week 50

Absolute change from randomization in patient-reported nighttime asthma symptom severity, as measured by a daily symptom diary (as defined in primary efficacy objective), at Week 50

Absolute change from randomization in patient-reported number of puffs of short-acting rescue inhaler or number of times nebulizer was used at Week 50

Absolute and relative change from randomization visit in the provocative concentration of methacholine causing a 20% drop in FEV, (PC20) as a measure of airway hyperresponsiveness at Week 30, in patients who consent to this optional assessment at select sites

D. Safety Objective

The safety objective for this study is to evaluate the safety of MTPS9579A compared with placebo on the basis of the following endpoints:

Incidence and severity of adverse events, with severity determined according to the WHO Toxicity Grading Scale

Change from randomization visit in physical examination findings

Change from randomization visit in vital signs

Change from randomization visit in ECG parameters

Change from randomization visit in clinical laboratory results

E. Pharmacokinetic Objectives

The pharmacokinetic (PK) objective for this study is to characterize the MTPS9579A PK profile on the basis of the following endpoint:

Serum concentration of MTPS9579A at specified timepoints

The exploratory PK objective for this study is to characterize concentrations of MTPS9579A in nasal mucosal lining fluid and to evaluate potential relationships between drug exposure and the efficacy and safety of MTPS9579A on the basis of the following endpoints:

Relationship between serum concentration, nasal mucosal lining fluid concentration, or PK parameters for MTPS9579A and efficacy or pharmacodynamic (PD) endpoints

Relationship between serum concentration, nasal mucosal lining fluid concentration, or PK parameters for MTPS9579A and safety endpoints

F. Immunogenicity Objectives

The immunogenicity objective for this study is to evaluate the immune response to MTPS9579A on the basis of the following endpoint:

Prevalence of anti-drug antibodies (ADAs) during the study relative to the prevalence of ADAs at the randomization visit

The exploratory immunogenicity objective for this study is to evaluate potential effects of ADAs on the basis of the following endpoint:

Relationship between ADA status and efficacy, safety, or PK/PD endpoints

G. Biomarker Objective

The exploratory biomarker objective for this study is to identify and/or evaluate biomarkers that are predictive of response to MTPS9579A (i.e., predictive biomarkers), can provide evidence of MTPS9579A activity (i.e., PD biomarkers), or can increase the knowledge and understanding of disease biology and drug safety, on the basis of the following endpoints:

Change from the randomization visit in biomarker levels in nasal mucosal lining fluid, urine, and serum samples

Relationship among biomarker levels in nasal mucosal lining fluid, urine, and serum samples

Relationship between biomarkers in nasal mucosal lining fluid, urine, and serum and efficacy, safety, PK, and immunogenicity endpoints

Rate of asthma exacerbations within subgroups defined by blood eosinophils during the 48-week double-blind treatment period

Time to first CompEx event (as defined in primary efficacy objective) within subgroups defined by blood eosinophils during the 48-week double-blind treatment period

Rate of asthma exacerbations in subgroups defined by mutations in the genes encoding tryptase (TPSAB1 and TPSB2) during the 48-week double-blind treatment period

Time to first CompEx event (as defined in primary efficacy objective) in subgroups defined by mutations in the genes encoding tryptase (TPSAB1 and TPSB2) during the 48-week double-blind treatment period 2. Study Design

A. Description of Study This is a Phase Ila, randomized, placebo-controlled, double-blind, multicenter, two-arm study of MTPS9579A compared with placebo as an add-on therapy in patients with uncontrolled moderate to severe asthma who are receiving daily ICS therapy and at least one of the following additional controller medications: long-acting β-agonist (LABA), leukotriene modulator (leukotriene modifier (LTM) or leukotriene receptor antagonist (LTRA)), long-acting muscarinic antagonist (LAMA), or long-acting theophylline preparation. The study will randomize approximately 160 patients at approximately 55 sites globally.

This study will consist of a 12-28 day screening period, a 2-week single-blind placebo run-in period, a 48-week double-blind treatment period, and a safety follow-up visit at Week 52. During the screening period, patients must demonstrate acceptable inhaler, peak flow meter, and spirometry techniques, in addition to compliance with required, twice-daily use of an electronic diary (eDiary) for answering questions related to asthma symptoms, PEFR, and short-acting rescue therapy use. Patients who fail to meet eligibility criteria during the screening period will be permitted to re-screen once.

Patients who meet enrollment criteria for the run-in period will receive one single-blind dose of placebo (Week 0) to allow for the evaluation of variability in asthma control. At the randomization visit (Week 2), patients must meet additional eligibility criteria for the double-blind treatment period that includes continued compliance with required, twice-daily use of the eDiary.

Patients will be randomized in a 1:1 ratio to receive MTPS9579A (1800 mg IV every 4 weeks) or placebo. Study drug will be administered by IV infusion at the randomization visit (Week 2), Week 6, and every 4 weeks thereafter through Week 46.

During the treatment period, twice-daily assessment of asthma-related symptoms, PEFR, and use of short-acting rescue therapy will continue to be performed at home and recorded in the eDiary. More detailed assessments, including spirometry and FeNO measurements, will be performed during scheduled site visits. All patients will undergo PK, biomarker, and ADA sampling. An additional exploratory efficacy assessment, the methacholine challenge test to assess airway reactivity, will be conducted at select sites in a subset of patients (approximately 20 patients/study arm) who consent to this option and meet additional eligibility criteria.

A planned interim analysis will take place once approximately 51 patients have experienced a CompEx event in the 48-week, double-blind treatment period. The expected timing of this planned interim analysis is approximately 60 weeks after the first patient is randomized. This interim analysis will be conducted for administrative purposes only (i.e., planning of future studies).

B. Screening Period

The screening period of up to 4 weeks is intended to allow sufficient time for a patient to meet all eligibility requirements. Patients must complete at least 12 days of the screening period to demonstrate eDiary compliance. Patients who are unable to complete assessments or meet eligibility requirements during the screening period will be permitted to be re-screened once for a total of up to two times, with the following exception: Patients who do not meet the requirement of morning pre-bronchodilator FEV1 of 40%-80% or post-bronchodilator reversibility of FEV1 (liters) of ≥12% and ≥200 mL are allowed up to two additional attempts to meet these two eligibility criteria within the screening period, but only if their morning prebronchodilator FEV1 was between 35% and 85%.

Patients who rescreen ≤6 weeks after Informed Consent Form completion must only repeat the assessments that triggered screen failure. Patients who rescreen >6 weeks after Informed Consent Form completion are required to repeat the consent process and all screening assessments except tuberculosis (TB) screening and hepatitis serologies. However, TB screening and hepatitis serologies should be repeated if the re-screening takes place >6 months after initial screening or if there is risk of exposure.

C. Run-In Period

Patients enrolled in the run-in period will receive one single-blind dose of placebo at the run-in visit (Week 0). eDiary compliance must continue to be demonstrated on at least 5 of 7 days during each of the 2 consecutive weeks of the run-in period. Patients who do not meet eligibility criteria for the double-blind treatment period will be discontinued from the study and are not eligible for rescreening.

D. Number of Patients

Approximately 160 patients with moderate to severe asthma will be randomized in this study (80 patients in each of 2 treatment arms).

3. Target Population

A. Inclusion Criteria

i. Inclusion Criteria for Enrollment in the Run-In Period

Signed Informed Consent Form

Ability to comply with the study protocol, in the investigator's judgment

Age 18-75 years, inclusive, at the time of signing the Informed Consent Form

Body mass index of 18-38 kg/m² and weight ≥40 kg at screening

Documented physician-diagnosed asthma for at least 12 months prior to screening

Pre-bronchodilator FEV1 40%-80% predicted at screening

Post-bronchodilator reversibility of FEV1 (liters) ≥12% and ≥200 mL at screening

FEV1forced vital capacity (FVC) <70%

Treatment with asthma controller therapy (daily ICS 100 μg of fluticasone propionate or equivalent) and at least one additional controller therapy (LABA, LAMA, LTM/LTRA)) for ≥3 months prior to screening, with no changes within 4 weeks prior to screening or during the screening period and no anticipated changes in controller dosing regimens throughout the study
For patients receiving a total daily ICS dose of <500 μg fluticasone propionate or equivalent, one of their additional controller therapies must be LABA.
For patients receiving a total daily ICS dose of ≥500 μg fluticasone propionate or equivalent, they must receive one or more of the following additional controller therapies: LABA, LTM/LTRA, LAMA, or long-acting theophylline preparations.

Asthma Control Questionnaire, 5-item version score ≥1.5 at screening

Documented history (e.g., medical report, pharmacy prescription assessed by investigator) of 1 asthma exacerbation within the 12 months prior to screening while on daily ICS maintenance therapy (same or higher dose as at screening), defined as new or increased asthma symptoms (wheezing, coughing, dyspnea, chest tightness, and/or nighttime awakenings due to these symptoms) that result in at least one of the following:
Hospitalization or an emergency department or urgent care visit with systemic corticosteroid treatment
Use of systemic (IV, IM, or oral) corticosteroids for ≥3 days or a long-acting depot corticosteroid preparation with a therapeutic effectiveness of ≥3 days

Demonstration of acceptable inhaler, peak flow meter, and spirometry techniques at screening

Demonstrated compliance with required use of the eDiary, defined as completing all required assessments (answering questions related to asthma symptoms, PEFR measurements, and use of short-acting rescue therapy) on at least 5 of 7 days during each of 2 consecutive weeks within the screening period (12-28 days)

For women of childbearing potential: agreement to remain abstinent (refrain from heterosexual intercourse) or use contraception

For men: agreement to remain abstinent (refrain from heterosexual intercourse) or use a condom, and agreement to refrain from donating sperm

ii. Inclusion Criteria for Enrollment in the Double-Blind Treatment Period

After completing the run-in period, patients must meet the following additional criteria for enrollment in the double-blind treatment period:

Demonstrated compliance with required use of the eDiary, defined as completing all required assessments (answering questions related to asthma symptoms, PEFR measurements, and use of short-acting rescue therapy) on at least 5 of 7 days during each of 2 consecutive weeks during the run-in period

No changes in ICS therapy or allowed controller medications during the run-in period

No new asthma exacerbation or infection during the run-in period

iii. Inclusion Criteria for the Optional Methacholine Challenge Test

Patients enrolled in the double-blind treatment period must meet the following additional criteria for enrollment into the optional methacholine challenge test:

Signed Informed Consent Form for the Optional Methacholine Challenge Test

Methacholine challenge at randomization (PC20≤8 mg/mL)

FEV1 60%-80% predicted

B. Exclusion Criteria

Patients who meet any of the following criteria will be excluded from study entry:

History or evidence of vocal cord dysfunction, reactive airways dysfunction syndrome, hyperventilation associated with panic attacks, or other mimics of asthma

History or evidence of significant respiratory disease other than asthma, including occupational asthma, aspirin-sensitive asthma, asthma-chronic obstructive pulmonary disease (COPD) overlap syndrome, bronchiolitis, interstitial lung disease, or COPD

Current smoker, former smoker with smoking history of >10 pack-years, or unwilling to abstain from smoking from the time of consent through the completion of the study
A current smoker is defined as someone who has smoked tobacco or marijuana products on at least 30 days within the 24 months prior to Visit 1.
A patient who smokes occasionally (smoked a tobacco or marijuana product on fewer than 30 days within the 24 months prior to Visit 1) and has a total smoking history of 10 pack-years may be permitted but must agree to abstain from all smoking during the study.
A pack-year is defined as the average number of packs of cigarettes per day times the number of years of smoking.

History or evidence of substance abuse that, in the investigator's judgment, would affect the patient's ability to participate in the study, pose a risk to patient safety, interfere with the conduct of the study, or have an impact on the study results

History or evidence of any clinically significant medical condition/disease (e.g., psychiatric, neurologic, cardiovascular, renal, hepatic, gastrointestinal, endocrine, autoimmune) or abnormalities in laboratory tests that, in the investigator's judgment, precludes the patient's safe participation and completion of the study, or interferes with the conduct and interpretation of the study
Patients with well-controlled comorbid disease on a stable treatment regimen for 4 weeks prior to screening are eligible for the study.

Hemoglobin A1c (HbA1c) >8.5% at screening or any other clinically significant finding that, in the opinion of the investigator, may define uncontrolled diabetes

Myocardial infarction, unstable angina pectoris, or stroke within 12 months prior to screening

Any chronic heart failure exacerbation within 12 months prior to screening or at risk for heart failure exacerbation in the investigator's opinion

History or presence of an abnormal ECG that is clinically significant in the investigator's opinion, including complete left bundle branch block, second- or third-degree atrioventricular heart block, or evidence of prior myocardial infarction

QT interval corrected through use of Fridericia's formula (QTcF)>450 ms, if patient is male, or QTcF>470, if patient is female, demonstrated by at least two ECGs>30 minutes apart

Active malignancy or history of malignancy within 5 years of screening, except for appropriately treated non-melanoma skin carcinoma, cervical carcinoma in situ, breast ductal carcinoma in situ, or Stage I uterine cancer

Positive for hepatitis C virus (HCV) antibody at screening, unless HCV RNA<15 IU/mL (or undetectable) at screening and for 6 months if successfully completed HCV anti-viral treatment

Positive hepatitis B surface antigen (HBsAg) at screening, or Negative HBsAg and positive hepatitis B core antibody (HBcAb)

Positive HIV antibody at screening

Positive for TB during screening, defined as either a positive purified protein derivative (PPD) test 5 mm of induration 48-72 hours after injection) or a positive QUANTIFERON® TB Gold (QFT-G) test during screening

For patients with a history of BCG vaccination, the following criteria for the QFT-G apply:
An indeterminate QFT-G should be repeated.
A positive QFT-G or two successive indeterminate QFT-G results should be considered a positive diagnostic TB test.
An indeterminate QFT-G followed by a negative QFT-G test should be considered a negative diagnostic TB test.
Patients with a positive PPD test or QFT-G are eligible if they meet all of the following criteria:
   No symptoms consistent with TB
   Documented history of a completed course of adequate prophylaxis (completed treatment for latent TB per the treatment options as stated in the WHO guidelines) prior to screening
   No known exposure to a case of active TB after most recent prophylaxis
   No evidence of active TB on chest radiograph within 3 months prior to screening

Acute infection requiring either surgical intervention (e.g., drainage) or medical therapy (e.g., antibiotics) within 4 weeks prior to screening

Active parasitic infection within 6 months prior to screening

Planned surgical intervention during the course of the study

History of any known immunodeficiency disorder

History of documented immune complex disease (Type III hypersensitivity reactions) to monoclonal antibody administration

History of anaphylaxis to any biologic therapy for any indication

Known sensitivity to any of the active substances or their excipients to be administered during dosing

Initiation of or change in allergen immunotherapy within 3 months prior to screening, during the screening period, or anticipated need during the course of the study

Treatment with immunoglobulin or blood products within 4 weeks prior to screening, during the screening period, or anticipated need during the course of the study

Treatment with any live or live, attenuated vaccines within 4 weeks prior to screening, during the screening period, or anticipated need during the course of the study

Treatment with any licensed biologic agent (e.g., omalizumab, mepolizumab, reslizumab, dupilumab) within 3 months or 5 drug half-lives prior to screening (whichever is longer), during the screening period, or anticipated need during the course of the study

Treatment with any investigational therapy within 3 months or 5 drug half-lives prior to screening (whichever is longer), during the screening period, or anticipated need during the course of the study

Maintenance oral corticosteroid therapy, defined as daily or alternate day oral corticosteroid maintenance therapy, within 3 months prior to screening or during the screening period

Treatment with systemic corticosteroids within 4 weeks (oral or IV) or 12 weeks (IM) prior to screening or during the screening period for any reason, including treatment for an acute exacerbation event

Treatment with intra-articular corticosteroids within 4 weeks prior to screening, during the screening period, or anticipated need during the course of the study

Maintenance oral or SC β-agonist therapy (e.g., terbutaline) within 2 weeks prior to screening, during the screening period, or anticipated need during the course of the study

Treatment with phosphodiesterase-4 inhibitors (e.g., roflumilast) within 4 weeks prior to screening, during the screening period, or anticipated need during the course of the study

Treatment with immunomodulatory, immunosuppressive (e.g., methotrexate, troleandomycin, oral gold, cyclosporine, azathioprine), or experimental anti-inflammatory therapy within 3 months or 5 drug half-lives prior to screening (whichever is longer), during the screening period, or anticipated need for these medications during the course of the study

Maintenance oral or inhaled antibiotic therapy within 2 weeks prior to screening, during the screening period, or anticipated need during the course of the study

Treatment with mast cell stabilizers (e.g., chromolyn) within 2 weeks prior to screening, during the screening period, or anticipated need during the course of the study

Treatment with homeopathic medications, herbal medications, acupuncture, or hypnosis for treatment of allergic disease within 2 weeks prior to screening, during the screening period, or anticipated need during the course of the study

Intubation for respiratory failure due to asthma within 12 months prior to screening

Maintenance intermittent positive pressure ventilation within 2 weeks prior to screening, during the screening period, or anticipated need during the course of the study

Maintenance bilevel positive airway pressure therapy within 2 weeks prior to screening, during the screening period, or anticipated need during the course of the study

Bronchial thermoplasty treatment within 24 months prior to screening, during the screening period, or anticipated need during the course of the study

Pregnant or breastfeeding, or intending to become pregnant during the study or within 42 days after the final dose of MTPS9579A
Women of childbearing potential must have a negative serum pregnancy test at screening and a negative urine pregnancy test result at the randomization visit.

4. End of Study

The end of this study is defined as the date when all patients have completed the study completion or early termination visit, or have otherwise been discontinued from the study. The total duration of this study for each patient is approximately 56 weeks, including screening, run-in, treatment, and follow-up. In addition, the Sponsor may decide to terminate the study at any time.

5. Length of Study

The total length of the study, from screening of the first patient to the end of the study, is expected to be approximately 25 months.

6. Investigational Medicinal Products

A. MTPS9579A and Placebo During the run-in period, patients will receive one single-blind dose of placebo (Week 0) to allow for evaluation of variability in asthma control. During the double-blind treatment period, MTPS9579A or placebo will be administered by IV infusion at the randomization visit (Week 2), Week 6, and every 4 weeks thereafter through Week 46.

B. Non-Investigational Medicinal Products

All patients must be on a stable asthma treatment regimen consisting of ICS therapy plus at least one additional controller medication. Refer to the local prescribing information for the formulation, packaging, and handling of these medications. Patients may not be on systemic (oral, IV, or IM) corticosteroids, biologic agents, or experimental therapeutics for the treatment of asthma.

7. Statistical Methods

A. Primary Analysis

The primary efficacy endpoint is time to first CompEx event, defined as time from randomization to first asthma exacerbation or diary worsening during the 48-week double-blind treatment period. Asthma exacerbations and diary worsening are defined as follows:

Asthma exacerbations are assessed by the investigator and defined as new or increased asthma symptoms (wheezing, coughing, dyspnea, chest tightness, and/or nighttime awakenings due to these symptoms) that result in one or both of the following:
Hospitalization or an emergency department or urgent care visit requiring administration of systemic corticosteroid treatment
Treatment with systemic (IV, IM, or oral) corticosteroids for ≥3 days or a long acting depot corticosteroid preparation with a therapeutic effectiveness of ≥3 days

Diary worsening is based on the occurrence of prespecified changes (deteriorations) in the following six parameters: morning PEFR, evening PEFR, morning symptom score, evening symptom score, morning short-acting rescue therapy use, and evening short-acting rescue therapy use. Deterioration criteria, defined as either a change from baseline (threshold) or worsening of a certain magnitude (slope) over 5 consecutive days, are presented for each parameter in the protocol. Diary worsening is defined as occurrence of one or both of the following scenarios:
Patient meets threshold deterioration criterion (i.e., prespecified change from baseline) for PEFR (morning and/or evening) and at least one other parameter (i.e., morning symptom score, evening symptom score, morning rescue therapy use, and/or evening rescue therapy use) on 2 consecutive days.
Patient meets threshold deterioration criterion (i.e., prespecified change from baseline) for one parameter on 2 consecutive days and slope deterioration criterion (i.e., prespecified change over 5 consecutive days calculated via univariate linear regression) for all six parameters.

For the purposes of determining whether the threshold deterioration criteria are met, baseline values for each of the six parameters will be calculated for each patient as the mean over the 10 days ending just before the day of randomization.

In the event that the first diary worsening scenario is met (i.e., threshold met in two parameters), the diary worsening event will start on the first of the 2 consecutive days (defined as Event Days 0 and 1).

In the event that the second diary worsening scenario is met (i.e., threshold in one parameter and slope in all six parameters), the diary worsening event will start on the first of the 2 consecutive days that the threshold was met (Event Days 0 and 1), and the slope criteria for the six parameters must be met on Day 0 and the 4 consecutive days prior to that day (i.e., Event Day -4 through Event Day 0).

To qualify for the second diary worsening scenario, data from at least 3 of the 5 consecutive days must be available for calculation of the slope for each parameter. Analyses will be based on observed asthma exacerbations and diary worsenings, with no imputation for premature discontinuation or missing diary entries.

The primary endpoint will be analyzed through use of a Cox proportional hazards regression model comparing MTPS9579A with placebo with respect to time to first CompEx event, with adjustment for baseline covariates. Estimated hazard ratios and their associated 95% confidence intervals will be provided.

B. Determination of Sample Size

The primary goal of this trial is estimation rather than hypothesis testing. This is largely due to the uncharacterized distribution of CompEx in the placebo arm. The interim analysis based upon 51 events will yield reasonable precision for estimating the true underlying hazard ratio. For example, an observed hazard ratio of 0.55 corresponds to a 95% confidence interval of 0.32 to 0.95. Randomizing 160 patients will enable observation of 51 CompEx events approximately 60 weeks after the first patient is enrolled assuming an exponential distribution, a placebo median time to first CompEx event of 22.6 weeks, and a hazard ratio of 0.55. This is further based upon an assumed enrollment rate of 0.25 patients per site per month, with 10% of the sites ready at study initiation and 75% of the sites active by 6 months after the first patient is enrolled.

C. Interim Analysis

An interim analysis will take place after approximately 51 patients have experienced a CompEx event in this study. The expected timing of the interim analysis will be approximately 60 weeks after the first patient is randomized. No formal stopping rules or decision criteria have been defined for the result of the interim analysis.

Access to treatment assignment information will follow the Sponsor's standard procedures. Given the hypothesis-generating nature of this study, the Sponsor may choose to conduct up to two additional interim efficacy analyses.

8. Method of Treatment Assignment and Blinding

After successful completion of the run-in period, at the Week 2 visit, patients will be randomly allocated to treatment with MTPS9579A or placebo at a 1:1 ratio through an interactive voice or web-based response system. Randomization will be stratified by region (United States/Western Europe vs. Eastern Europe vs. Southern Hemisphere) and number of prior asthma exacerbations (1 vs. ≥2) requiring use of systemic corticosteroids in the previous 12 months to balance patients across study arms. Enrollment caps for blood eosinophil level (Visit 1<150 vs. 150-300 vs.>300 cells/μL) of 35% per strata during randomization will be utilized to ensure a natural distribution of patients in both study arms. A permuted block randomization method will be employed.

9. Permitted Asthma Therapy

All patients will continue on a stable asthma treatment regimen, as outlined below:

Daily ICS therapy plus at least one additional controller medication (LABA, LAMA, LTM/LTRA) for ≥3 months prior to screening, with no changes within 4 weeks prior to screening or during the screening period and no anticipated changes in controller dosing regimens throughout the study
For patients receiving a total daily ICS dose of <500 μg fluticasone propionate or equivalent, one of their additional controller medications must be LABA.
For patients receiving a total daily ICS dose of 500 μg fluticasone propionate or equivalent, they must receive one or more of the following additional controller medications: LABA, LTM/LTRA, LAMA, or long-acting theophylline preparations.

Any changes to the formulation or dose of ICS or any additional controller medications should be avoided, with the exception of the theophylline dose, which may be adjusted as appropriate on the basis of serum theophylline levels. If changes to the ICS brand or formulation are unavoidable, the patient may be switched to another ICS brand or formulation at a dose equivalent to the ICS dose that the patient was receiving at study entry.

In order to maintain stable controller medication doses, patients may not use an ICS/LABA combination inhaler (i.e., single maintenance and reliever therapy) as rescue therapy. Note: This only pertains to rescue use; ICS/LABA as a stable controller medication is permitted.

It is expected that the majority of patients will be using short-acting β-agonist (SABA) or short-acting muscarinic antagonist (SAMA) therapy for symptoms of uncontrolled asthma per existing treatment guidelines. Combination SABA or SAMA inhalers (e.g., albuterol/ipratropium) are also permitted. Short-acting rescue therapy must be administered via the patient's prescribed inhaler or nebulizer.

Patients who require any systemic corticosteroids within 4 weeks (oral or IV) or 12 weeks (IM) prior to screening or during the screening or run-in periods will not be eligible for the trial. The use of systemic corticosteroids is permitted for acute asthma management after randomization.

10. Assessments Completed by the Patient at Home

At the initial screening visit, patients will receive an eDiary and a peak flow meter to measure PEFR. Patients will be instructed in eDiary use and asked to use the eDiary twice per day (morning/evening) to record asthma-related symptoms, PEFR, and use of short-acting rescue therapy. The eDiary will remind patients twice daily to complete their entries and will provide a time window during which the entry must be completed at approximately the same time each day. Patients will use the eDiary during screening and through Week 50.

Compliance with the required use of the eDiary and PEFR measurements must be demonstrated on 5 of 7 days during each of 2 consecutive weeks during the screening period and also during the 2 week run-in period. eDiary compliance less than 70% (fewer than 5 out of 7 days/week) during the screening period will result in screen failure. eDiary compliance less than 70% (fewer than 5 out of 7 days/week) during the run-in period will result in study discontinuation. Site staff will review daily diary compliance at each subsequent visit and provide refresher training if compliance is consistently less than 70% between study visits.

The daily diary comprises:

Daytime/nighttime asthma symptoms

Nighttime awakenings

Number of doses of short-acting rescue therapy

11. Study and Dose Rationale

The current high unmet medical need in asthma is for patients with uncontrolled disease despite adherence to guidelines-based, standard-of-care therapy. In this study, the target population is patients with moderate to severe asthma whose disease remains uncontrolled despite daily use of ICS therapy and at least one additional controller medication. Patients must have a diagnosis of asthma, an Asthma Control Questionnaire, 5-item version (ACQ-5) score≥1.5, and have experienced at least one asthma exacerbation within the 12 months prior to screening as evidence of uncontrolled disease.

The rationale for conducting this Phase Ila proof-of-activity study of MTPS9579A in patients with asthma requiring ICS and a second controller is to evaluate efficacy, safety, pharmacokinetics, and pharmacodynamics in a relevant patient population. Inhibiting tryptase with MTPS9579A is anticipated to block airway inflammation downstream of mast cell activation across all asthma types. The goals of this study are to determine the impact of monthly treatment with MTPS9579A on patients' signs and symptoms of asthma using a combination of patient-reported measures and functional measures of exacerbation and to continue to understand safety and the PK/PD relationships of MTPS9579A and tryptase. The rationale for the optional methacholine challenge test is that mast cell microlocalization within airway smooth muscle cell bundles is thought to contribute to airway hyper-responsiveness. Methacholine challenge testing, a measure of airway hyper-responsiveness, will be used to demonstrate physiological activity of MTPS9579A in patients.

The dose of MTPS9579A in this study (1800 mg IV Q4W) has been selected based on the totality of the following data: understanding of tryptase biology in healthy volunteers and patients with asthma, Phase Ia/b SAD/MAD clinical trial data, MTPS9579A properties, mechanism of action, nonclinical activity and safety, and prior clinical experience targeting tryptase. The selected Phase IIa dose is within the dose range previously evaluated in the Phase Ia/b study (GA40396). In that study, doses up to 3600 mg of MTPS9579A administered intravenously as a single dose or as a Q4W regimen (3 doses), which was the maximum dose tested, was well tolerated. The dose and regimen to be tested in this study (1800 mg IV Q4W) is projected to maximize the potential for clinical benefit based on the totality of data available to date. At steady-state concentrations, it is anticipated that this dose will reduce active tryptase levels by 95%, which accounts for the maximum concentration of active tryptase that may be present in the airway of patients with asthma while ensuring patient safety.

### Other Aspects

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating a patient having asthma, the method comprising administering to a patient having asthma an anti-tryptase beta antibody in a dosing regimen comprising a dosing cycle, wherein the dosing cycle comprises a first dose (C1D1 ) of the anti-tryptase beta antibody selected from 300 mg intravenously (IV), 450 mg IV, 750 mg SC, 900 mg IV, 1350 mg IV, 1800 mg IV, or 3600 mg IV, wherein the anti-tryptase beta antibody comprises the following six complementarity determining regions (CDRs):
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

2. The method of claim 1, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

3. The method of claim 2, wherein the VH domain comprises the amino acid sequence of SEQ ID NO: 7.

4. The method of claim 2, wherein the VL domain comprises the amino acid sequence of SEQ ID NO: 8.

5. The method of claim 2, wherein the VH domain comprises the amino acid sequence of SEQ ID NO: 7 and the VL domain comprises the amino acid sequence of SEQ ID NO: 8.

6. The method of any one of claims 1-5, wherein the antibody comprises (a) a heavy chain comprising the amino acid sequence of SEQ ID NO: 9 and (b) a light chain comprising the amino acid sequence of SEQ ID NO: 10.

7. The method of any one of claims 1-5, wherein the antibody comprises (a) a heavy chain comprising the amino acid sequence of SEQ ID NO: 11 and (b) a light chain comprising the amino acid sequence of SEQ ID NO: 10.

8. The method of any one of claims 1-7, wherein the C1D1 is 300 mg IV.

9. The method of any one of claims 1-7, wherein the C1D1 is 450 mg IV.

10. The method of any one of claims 1-7, wherein the C1D1 is 750 mg SC.

11. The method of any one of claims 1-7, wherein the C1D1 is 900 mg IV.

12. The method of any one of claims 1-7, wherein the C1D1 is 1350 mg IV.

13. The method of any one of claims 1-7, wherein the C1D1 is 1800 mg IV.

14. The method of any one of claims 1-7, wherein the C1D1 is 3600 mg IV.

15. The method of any one of claims 1-14, wherein the dosing cycle further comprises a second dose (C1D2) and a third dose (C1D3) of the anti-tryptase beta antibody, wherein the C1D2 and the C1D3 are each equal to the C1D1.

16. The method of claim 15, wherein the doses of the dosing cycle are administered to the subject every four weeks (q4w).

17. The method of claim 15 or 16, wherein the dosing cycle has a length of about 57 days.

18. The method of claim 17, wherein the C1D1 is administered on Day 1 of the dosing cycle, the C1D2 is administered on Day 29 of the dosing cycle, and the C1D3 is administered on Day 57 of the dosing cycle.

19. The method of any one of claims 15-18, wherein the dosing regimen consists of one dosing cycle.

20. A method of treating a patient having asthma, the method comprising administering to a patient having asthma an anti-tryptase beta antibody in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

21. The method of any one of claims 1-20, wherein the asthma is moderate asthma, severe asthma, allergic asthma, or atopic asthma.

22. The method of claim 21, wherein the severe asthma is uncontrolled despite standard-of-care therapy.

23. The method of any one of claims 1-20, wherein the asthma is moderate to severe asthma.

24. The method of any one of claims 1-23, wherein the patient is receiving daily inhaled corticosteroid therapy and at least one of the following controller medications: a long-acting β-agonist (LABA), a leukotriene modulator, a long-acting muscarinic antagonist (LAMA), or a long-acting theophylline preparation.

25. The method of claim 24, wherein the leukotriene modulator is a leukotriene modifier (LTM) or leukotriene receptor antagonist (LTRA).

26. A kit comprising an anti-tryptase beta antibody and instructions to administer the anti-tryptase beta antibody to a patient having asthma in accordance with the method of any one of claims 1-25.

27. An anti-tryptase beta antibody for use in treating a patient having asthma, wherein the anti- tryptase beta antibody is for administration to a patient having asthma in a dosing regimen comprising a dosing cycle, wherein the dosing cycle comprises a first dose (C1D1 ) of the anti-tryptase beta antibody selected from 300 mg IV, 450 mg IV, 750 mg SC, 900 mg IV, 1350 mg IV, 1800 mg IV, or 3600 mg IV, wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

28. The anti-tryptase beta antibody for use of claim 27, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

29. The anti-tryptase beta antibody for use of claim 28, wherein the VH domain comprises the amino acid sequence of SEQ ID NO: 7.

30. The anti-tryptase beta antibody for use of claim 28, wherein the VL domain comprises the amino acid sequence of SEQ ID NO: 8.

31. The anti-tryptase beta antibody for use of claim 28, wherein the VH domain comprises the amino acid sequence of SEQ ID NO: 7 and the VL domain comprises the amino acid sequence of SEQ ID NO: 8.

32. The anti-tryptase beta antibody for use of any one of claims 27-31, wherein the antibody comprises (a) a heavy chain comprising the amino acid sequence of SEQ ID NO: 9 and (b) a light chain comprising the amino acid sequence of SEQ ID NO: 10.

33. The anti-tryptase beta antibody for use of any one of claims 27-31, wherein the antibody comprises (a) a heavy chain comprising the amino acid sequence of SEQ ID NO: 11 and (b) a light chain comprising the amino acid sequence of SEQ ID NO: 10.

34. The anti-tryptase beta antibody for use of any one of claims 27-33, wherein the C1D1 is 300 mg IV.

35. The anti-tryptase beta antibody for use of any one of claims 27-33, wherein the C1D1 is 450 mg IV.

36. The anti-tryptase beta antibody for use of any one of claims 27-33, wherein the C1D1 is 750 mg SC.

37. The anti-tryptase beta antibody for use of any one of claims 27-33, wherein the C1D1 is 900 mg IV.

38. The anti-tryptase beta antibody for use of any one of claims 27-33, wherein the C1D1 is 1350 mg IV.

39. The anti-tryptase beta antibody for use of any one of claims 27-33, wherein the C1D1 is 1800 mg IV.

40. The anti-tryptase beta antibody for use of any one of claims 27-33, wherein the C1D1 is 3600 mg IV.

41. The anti-tryptase beta antibody for use of any one of claims 27-40, wherein the dosing cycle further comprises a second dose (C1D2) and a third dose (C1D3) of the anti-tryptase beta antibody, wherein the C1D2 and the C1D3 are each equal to the C1D1.

42. The anti-tryptase beta antibody for use of claim 41, wherein the doses of the dosing cycle are administered to the subject every four weeks (q4w).

43. The anti-tryptase beta antibody for use of claim 41 or 42, wherein the dosing cycle has a length of about 57 days.

44. The anti-tryptase beta antibody for use of claim 43, wherein the C1D1 is administered on Day 1 of the dosing cycle, the C1D2 is administered on Day 29 of the dosing cycle, and the C1D3 is administered on Day 57 of the dosing cycle.

45. The anti-tryptase beta antibody for use of any one of claims 41-44, wherein the dosing regimen consists of one dosing cycle.

46. An anti-tryptase beta antibody for use in treating a patient having asthma, wherein the anti- tryptase beta antibody is for administration to a patient having asthma in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

47. The anti-tryptase beta antibody for use of any one of claims 27-46, wherein the asthma is moderate asthma, severe asthma, allergic asthma, or atopic asthma.

48. The anti-tryptase beta antibody for use of claim 47, wherein the severe asthma is uncontrolled despite standard-of-care therapy.

49. The anti-tryptase beta antibody for use of any one of claims 27-48, wherein the asthma is moderate to severe asthma.

50. The anti-tryptase beta antibody for use of any one of one of claims 27-49, wherein the patient is receiving daily inhaled corticosteroid therapy and at least one of the following controller medications: an LABA, a leukotriene modulator, an LAMA, or a long-acting theophylline preparation.

51. The anti-tryptase beta antibody for use of claim 50, wherein the leukotriene modulator is an LTM or an LTRA.

52. Use of an anti-tryptase beta antibody in the manufacture of a medicament for treating a patient having asthma, wherein the medicament is for administration to a patient having asthma in a dosing regimen comprising a dosing cycle, wherein the dosing cycle comprises a first dose (C1D1 ) of the anti-tryptase beta antibody selected from 300 mg IV, 450 mg IV, 750 mg SC, 900 mg IV, 1350 mg IV, 1800 mg IV, or 3600 mg IV, wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

53. Use of an anti-tryptase beta antibody in the manufacture of a medicament for treating a patient having asthma, wherein the medicament is for administration to a patient having asthma in a dosing regimen comprising a dosing cycle, wherein the dosing cycle comprises administering 1800 mg IV of the anti-tryptase beta antibody to the patient every four weeks (q4w), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).
